(19) United States
(12) Patent Application Publication
(10) Pub. No.: US 2024/0410014 A1
(43) Pub. Date: Dec. 12, 2024

(54) GENE SIGNATURE OF CTCS TO DETECT MELANOMA BRAIN METASTASIS

(71) Applicants: Dario Marchetti, Albuquerque, NM (US); Tetiana Bowley, Albuquerque, NM (US)

(72) Inventors: Dario Marchetti, Albuquerque, NM (US); Tetiana Bowley, Albuquerque, NM (US)

(21) Appl. No.: 18/678,737

(22) Filed: May 30, 2024

Related U.S. Application Data
(60) Provisional application No. 63/504,816, filed on May 30, 2023.

(51) Int. Cl. C12Q 1/6886; C12Q 1/6874

(52) U.S. Cl. CPC C12Q 1/6886; C12Q 1/6874; C12Q 2600/158

(57) ABSTRACT

Provided herein are methods to detect in a mammal having or at risk of having melanoma a risk of brain metastasis, methods of treating, compositions, kits and animal models.

### PRIORITY

This application claims the benefit of priority of U.S. Provisional Patent Application No. 63/504,816, filed on May 30, 2023, and is incorporated by reference herein in its entirety.

### STATEMENT OF GOVERNMENT RIGHTS

This invention was made with government support under grants R01 CA21699 awarded by the National Institutes of Health and P30CA118100-16 awarded by the National Cancer Institutes. The government has certain rights in the invention.

### INCORPORATION BY REFERENCE OF SEQUENCE LISTING

This application contains a Sequence Listing which has been submitted electronically in ST26 format and hereby incorporated by reference in its entirety. Said ST26 file, created on May 29, 2024, is name 1863288US1.xml and is 127,779 bytes in size.

### BACKGROUND

Melanoma is the most aggressive and lethal skin cancer, and the one with highest propensity to generate brain metastasis (MBM; Eroglu et al., 2019; Johnson & Young, 1996; Biermann et al., 2022). MBM is diagnosed clinically in up to 60% of patients with metastatic melanoma and in up to 80% of patients at autopsy. A poor prognosis (4-6 months survival), and extreme deterioration in quality of life have been reported for patients with MBM (Eroglu et al., 2019; Fischer et al., 2019; In et al., 2020; Sperduto et al., 2020; Berghoff et al., 2016; Gonzalez et al., 2022). The high mortality rate of patients with MBM is linked to brain tumor expansion, hemorrhage, increased intracranial and extracranial pressure (Berghoff et al., 2016; Kircher et al., 2016). At time of autopsy, the tumor mass is often larger than clinical imaging suggests (Kircher et al., 2016). Local therapies include resection of a single MBM lesion, if surgically accessible, and radiation (Kircher et al., 2016; Wronski et al., 1995). Other therapeutic interventions include systemic therapies, such as targeted or immune-based therapies (Kircher et al., 2016; Luke et al., 2017). While checkpoint inhibitors have yielded some promising results treating patients with MBM (Eroglu et al., 2019; Sperduto et al., 2020; Berghoff et al., 2016; Chan et al., 2017), clinical activity in the brain is significantly less than in extracranial metastasis.

Metastasis is a complex multistep process enabling the spread of tumor cells from a primary tumor to distant organs, resulting in poor prognosis and high morbidity (Kircher et al., 2016; Nguyen 2022). Specifically, melanoma cells have the capability to metastasize to most organs, with most common sites being the lungs, skin, liver, and brain (Eroglu et al., 2019). The brain microenvironment represents a unique niche due to the selective semipermeable blood-brain barrier, high nutrient and energy consumption, and immune privilege (Kircher et al., 2016; Zhang & Yu, 2011). Circulating tumor cells (CTC) are “seeds” of fatal metastatic disease and smallest functional units of cancer. CTCs disseminate from primary and/or metastatic tumors into vasculature and initiate tumor development at distant organs (Gupta & Massague, 2006; Dianat-Moghadam et al., 2020; Alix-Panabieres & Pantel, 2014). Only a small fraction of CTCs can successfully develop into metastasis/MBM, due to the harsh physical, oxidative, and other microenvironmental stresses they encounter in blood (Micalizzi et al., 2017; Werner-Klein et al., 2018). Extensive reports have also demonstrated that CTC dissemination occurs early, and that CTCs migrate to distant organs where they can initiate metastasis or remain dormant (Dianat-Moghadam et al., 2020; Jones et al., 2013). Importantly, cancer progression and clinical outcomes of patients with melanoma directly correlate with numbers of CTCs in the bloodstream (Lucci et al., 2020).

### SUMMARY

Melanoma brain metastasis (MBM) is linked to poor prognosis and low overall survival. It was hypothesized that melanoma circulating tumor cells (CTC) possess a gene signature significantly expressed and associated with MBM. Employing a multipronged approach, a common CTC gene signature for ribosomal protein large/small subunits (RPL/RPS) was identified which associates with MBM onset and progression. Experimental strategies involved capturing, transcriptional profiling, and interrogating CTCs, either directly isolated from blood of patients with melanoma at distinct stages of MBM progression or from CTC-driven MBM in experimental animals. An MRI CTC-derived MBM xenograft model (MRI-MBM CDX) was developed to discriminate MBM spatial and temporal growth, recreating MBM clinical presentation and progression. Further, comprehensive transcriptional profiling of MRI-MBM CDXs, along with longitudinal monitoring of CTCs from CDXs possessing and/or not possessing MBM, was performed.

The findings suggest that enhanced ribosomal protein content/ribogenesis may contribute to MBM onset. Because ribosome modifications drive tumor progression and metastatic development by remodeling CTC translational events, overexpression of the CTC RPL/RPS gene signature could be implicated in MBM development. Collectively, this study provides insights for relevance of the CTC RPL/RPS gene signature in MBM and identify potential targets for therapeutic intervention to improve patient care for patients with melanoma diagnosed with or at high risk of developing MBM.

In one embodiment, a method to detect in a mammal having or at risk of melanoma a risk of brain metastasis is provided comprising: providing a sample from the mammal having circulating tumor cells (CTCs); detecting the presence or amount of expression of two or more genes in the CTCs; and determining whether the presence or amount is indicative of melanoma brain metastases (MBM). In one embodiment, the mammal is a human. In one embodiment, the mammal has melanoma. In one embodiment, the sample is a physiological fluid sample. In one embodiment, the sample is a blood sample. In one embodiment, the CTCs are human Mel-A⁺ (CD146). In one embodiment, the CTCs are CD45⁻, CD235⁻, CD34⁻, CD73⁻, CD90⁻, CD105⁻, or any combination thereof. In one embodiment, the presence or amount is increased relative to a corresponding sample from a corresponding mammal without MBM. In one embodiment, the presence or amount is indicative of onset of MBM. In one embodiment, the presence or amount is indicative of progression of MBM. In one embodiment, an increase in expression of at least one of the genes is indicative of MBM. In one embodiment, at least 3, 4, 5, 6, 7, 8, 9, 10 or more genes or proteins are detected. In one embodiment, a plurality of RPL 12, RPL 13, RPL 18A, RPL 19, RPL 23, RPL 26, RPL 35A, RPL 37, RPL 38, RPL 6, RPL 7, RPL 7A, RPS 12, RPS 15A, RPS 18, RPS 24, RPS 26, RPS 28, RPS 5, RPS 7, or RPS A, or any combination thereof, is detected. In one embodiment, a plurality of BIRC7, CDH3, CLK1, CSPG4, EIF4B, MRFAP1, PAIP1, PPDPF, RIMKLB, RPL12, RPL13, RPL18A, RPL19, RPL7, RPS12, RPS18, PRS24, PRS26, SPCS2, SPRY4, or any combination thereof, is detected. In one embodiment, RNA expression is detected. In one embodiment, protein expression is detected. In one embodiment, the method further comprises treating the mammal with a checkpoint inhibitor or a kinase inhibitor. In one embodiment, the inhibitor comprises pembrolizumab, nivolumab, atezolizumab, avelumab, durvalumab, or ipilimumab. In one embodiment, the method further comprises treating the mammal with an immunotherapy, stereotactic radiosurgery, surgical resection or whole-body radiotherapy.

Also provided is a kit or system for detecting gene expression of a plurality of RPL 12, RPL 13, RPL 18A, RPL 19, RPL 23, RPL 26, RPL 35A, RPL 37, RPL 38, RPL 6, RPL 7, RPL 7A, RPS 12, RPS 15A, RPS 18, RPS 24, RPS 26, RPS 28, RPS 5, RPS 7, or RPS A, or any combination thereof; or a plurality of BIRC7, CDH3, CLK1, CSPG4, EIF4B, MRFAP1, PAIP1, PPDPF, RIMKLB, RPL12, RPL13, RPL18A, RPL19, RPL7, RPS12, RPS18, PRS24, PRS26, SPCS2, SPRY4, or any combination thereof.

Further provided is a non-human mammalian model for MBM, comprising: a non-human mammal comprising human CTC cells. In one embodiment, the CTCs are human Mel-A⁺ (CD146). In one embodiment, the CTCs are CD45⁻, CD235⁻, CD34⁻, CD73⁻, CD90⁻, CD105⁻, or any combination thereof. In one embodiment, the CTCs express a plurality of RPL 12, RPL 13, RPL 18A, RPL 19, RPL 23, RPL 26, RPL 35A, RPL 37, RPL 38, RPL 6, RPL 7, RPL 7A, RPS 12, RPS 15A, RPS 18, RPS 24, RPS 26, RPS 28, RPS 5, RPS 7, or RPS A, or any combination thereof. In one embodiment, the CTCs express a plurality of BIRC7, CDH3, CLK1, CSPG4, EIF4B, MRFAP1, PAIP1, PPDPF, RIMKLB, RPL12, RPL13, RPL18A, RPL19, RPL7, RPS12, RPS18, PRS24, PRS26, SPCS2, SPRY4, or any combination thereof.

In one embodiment, the disclosure provides for a method to prevent, inhibit or treat a mammal having or at risk of melanoma brain metastasis, comprising: administering to the mammal a therapeutic composition, wherein CTCs in the mammal are detected as having increased expression of two or more genes. In one embodiment, the mammal is a human. In one embodiment, the CTCs have increased expression of a plurality of RPL 12, RPL 13, RPL 18A, RPL 19, RPL 23, RPL 26, RPL 35A, RPL 37, RPL 38, RPL 6, RPL 7, RPL 7A, RPS 12, RPS 15A, RPS 18, RPS 24, RPS 26, RPS 28, RPS 5, RPS 7, or RPS A, or any combination thereof. In one embodiment, the CTCs have increased expression of a plurality of BIRC7, CDH3, CLK1, CSPG4, EIF4B, MRFAP1, PAIP1, PPDPF, RIMKLB, RPL12, RPL13, RPL18A, RPL19, RPL7, RPS12, RPS18, PRS24, PRS26, SPCS2, SPRY4, or any combination thereof.

### BRIEF DESCRIPTION OF THE FIGURES

FIGS. 1A-1B. The capture, visualization, and enumeration of melanoma CTCs (MEL-PE⁺/DAPI⁺/CD34/CD45⁻ cells) from patients' blood using the CellSearch platform and CellSearch melanoma assay (Menarini Silicon Biosystems, Inc.). Peripheral blood (7.5 mL) was obtained from patients with primary (A) and metastatic (B) melanoma and analyzed by CellSearch. No CTCs were detected in these patient samples as MEL-PE⁺/DAPI⁺/CD34/CD45⁻ cells, according to CellSearch analyses. Cells from the human melanoma SK-Mel28 line (CellSearch melanoma CTC kit) were analyzed in parallel as positive control for (right). Displayed are the original CellSearch images using CellBrowser software (10× magnification).

FIGS. 2A-2C. Multiparametric flow cytometry gating for the isolation of viable Lin-negative/CTC-enriched populations from a number of independent patients with primary (A), and metastatic (B) melanoma. Enrichment of Lin-negative cell populations (CD45⁻/CD34⁻/CD73⁻/CD90⁻/CD105/CD235⁻ cells) was performed, as reported previously (Vishnoi et al., 2018). The same multiparametric FACS procedure was applied to healthy donor blood, showing no presence of Lin-negative cell population (negative control). C, Transcriptional profiling detailing discordance among Lin-positive (LinP) versus Lin-negative (LinN) cell populations, and LinN heterogeneity from independent patients with primary or metastatic (diagnosed with or without MBM) melanoma. Hierarchical clustering of gene expression profiling showing significant differences between the LinN (green) and LinP (red) cell populations isolated from primary, MBM, metastatic patients without MBM diagnosis (No MBM), and LinN cell populations isolated over time (0, 3, 6 months longitudinal collection) from a patient diagnosed with MBM and compared with LinP cells isolated from blood of healthy donors, respectively. Each LinN/LinP population is patient paired (same patient). Scatter plots show global gene expression of LinN cell populations with significant log 2 fold change (green dots), compared with LinP/healthy donor cell populations (red dots).

FIGS. 3A-3D. The generation of the MBM CTC xenograft model (MBM CDX). A, Immunodeficient (NSG) mice were injected intracardiacally with the MBM CTC-derived clone (5.0×10E5 70W-SM3-Luc2 cells), and subsequently imaged by IVIS to evaluate MBM onset with parallel pathologic examination. Consistent MBM (mice with yellow circles) was observed at 4 weeks postinjection (red arrow). B, Detection of CTC-driven MBM in 3 mice (circled in yellow) as early as 24 hours following CTC intracardiac injection. These mice were selected for longitudinal MRI MBM imaging (MRI-MBM CDXs). C, Parallel pathologic evaluation of CTC-injected mice detecting the presence of MBM along with metastasis to other organs (red arrows), reflecting the target organ metastatic specificity of clinical melanoma. D, 3D IVIS tomography of representative CTC MBM mice showing metastatic dissemination, notably to brain (MBM; red arrows).

FIGS. 4A-4B. A, Spatial and temporal MBM onset by MRI analyses using CDX mice (MRI-MBM CDXs). MRI-MBM CDXs underwent MRI analyses biweekly employing the Bruker 7-Tesla PET/MRI scanner. While no MBM was found at day 25 post-CTC injection, MRI detected the presence of MBM in all CDX mice at subsequent timepoints (day 39, day 46 after CTC injection) with specific MBM localization in the FL, PTL, and cerebellum regions (red arrows, yellow circles), reflecting the MBM presentation in patients. B, Spatial and temporal MRI analytic quantitation of MRI-MBM CDXs. Representative images of CTC-MBM CDXs employing the skull stripping procedure for removal of extra brain tissue to visualize brain tumors (left), brain atlas based MBM assessment showing alignment to 62 brain regions using ANTs Python program (middle), or T1W MRI displaying MBM sizes generated by the 3D Slicer software program (right).

FIGS. 5A-5C. Spatial and temporal MRI-MBM analyses of CDXs along with pathological assessment. The MRI-MBM detection in thalamic regions of the temporal and cerebellar regions of MRI-MBM CDXs was confirmed by 3D IVIS tomography showing MBM progression overtime (4-8 weeks, red arrows; A) and by pathologic evaluation for MBM presence in mice brain necropsies following MRI (B). Representative mouse brains with MBM (red arrows) are shown (C).

FIGS. 6A-6C. The capture and interrogation of CTCs from CDXs using the CTC Parsortix platform. Representative images of human melanoma CTCs captured/visualized by the CTC Parsortix platform, either as ex vivo single CTCs or homotypic CTC clusters from blood of MRI-MBM mice (N=3; A), patients with MBM (N=3; B), or as CTC-derived clonal cells (70W-SM3) spiked (positive control) in blood from healthy donors (negative control; N=3; C). CTCs were defined for absence of human FITC-CD45 (green fluorescence); however, presence of human Melan-A/Alexa Fluor 594 (red fluorescence), and DAPI staining within the separation Parsortix cassette. Human Melan-A⁺/DAPI⁺/CD45⁻ cells were then visualized and quantitated by confocal Zeiss LSM800 microscopy.

FIGS. 7A-7D. The hierarchical transcriptional classification of CTC-driven MBM. Regional specificity of CTC-driven MBM was detected in FL, temporal lobe, and cerebellum regions of CDXs (A), with a distinct MBM region-dependent transcriptional profiling/hierarchical clustering displaying unique gene expression patterns compared with uninjected CTC-derived clonal cell (70W-SM3-Luc2; B). Venn diagrams showing 263 upregulated (C) and 12 downregulated (D) genes as result of combinatorial gene expression analyses employing a four-pronged experimental approach consisting of transcriptome analyses of: (1) CTCs from MBM versus No MBM CDXs, (2) region-specific CTC MBM versus uninjected CTC-derived clonal cells, (3) LinN cells from MBM versus metastatic/primary patients, and (4) LinN cells longitudinally (0, 3, 6 months) isolated from a patient with MBM.

FIG. 8. The CTC RPL/RPS gene pathways of MBM. List of the top molecular pathways resulting from the four-pronged experimental approach and hierarchal clustering of MBM samples (Reactome pathway database). Highlighted in yellow are CTC translational pathways containing the CTC RPL/RPS gene signature of MBM.

FIGS. 9A-9B. CellSearch analyses of blood from healthy donors (normal blood), melanoma CTCderived clone 70W-SM3 spiked in blood, and human melanoma SK-Mel-28 cells. Normal blood from healthy donors was processed using CellSearch (upper left panel). No melanoma CTCs (MEL-PE+/DAPI+/CD45− cells) were captured. Spiked melanoma CTCderived clone (70W-SM3 cells) (lower left panels) and human melanoma SK-Mel-28 cells were used as respective positive controls (right panels) used as a positive control. Displayed are the original CellSearch images using CellBrowser™ software (10× magnification).

FIG. 10. Quantitation of IVIS analyses in CTC-derived clone-injected NGS mice. Total flux of the mouse brain region was measured by IVIS imaging system 24 hours following injection of CTC-Derived clonal cells (70W-SM3). Mice having MBM were subsequently processed were sent for MRI imaging (N=3), while mice with No MBM were subjected to IVIS imaging (N=7).

FIG. 11. Lung-targeting xenograft model of melanoma. Six NSG mice were injected with human melanoma cells (5.0×10E5 MeWo-Luc2 cells) and imaged by IVIS 24 hours later. No brain metastasis was detected in these mice (left panel). 3D IVIS tomography was performed biweekly to evaluate metastatic patterns in the animals.

FIG. 12. MRI imaging of female mice without MBM. Four NSG mice were injected with CTCderived clonal cells (5.0×10E5 70W-SM3-Luc2 cells) and processed for MRI imaging. MRI was performed biweekly using manganese contrast agent. No MBM were detected.

### DETAILED DESCRIPTION

Recent studies have identified a link between abnormal ribosome biogenesis and increased tumor burden (Elhamamsy et al., 2022; Li & Wang, 2020; Ebright et al., 2020; Bretones et al., 2018). For example, a study demonstrated that augmented expression of the ribosomal large-subunit protein 15 (RPL15) in breast cancer CTCs triggered massive metastatic spread and induced the translation of other ribosomal subunits proteins (Ebright et al., 2020). Accordingly, enhanced expression of ribosomal proteins results in ribosomopathies associated with metastatic development and progression (Elhamamsy et al., 2022; Li & Wang, 2020).

It was hypothesized that the comprehensive multilevel characterization of melanoma CTCs/Lin− cells isolated from patients (FACS sorted for absence of normal circulatory cells and Lin+ cells; Pauken et al., 2021) and/or CTC xenografts with and/or without MBM can identify biomarkers useful to evaluate effective therapies targeting and/or preventing MBM. Specifically, it was postulated that a common CTC genetic signature was uniquely associated with MBM onset and its progression over time. This was evaluated by performing complex multilevel analyses of CTCs correlating with MBM progression in patients with melanoma, additive to employing a novel MBM CTC xenograft model (MBM-CDX). Furthermore, MRI was used to detect the spatial and temporal progression of MBM in a newly developed preclinical model (MRI-MBM CDX).

A CTC RPL/RPS gene signature of MBM was identified which was found to be common in CTCs characterized from all MBM samples analyzed, either from patients or xenograft models (the term “RPL” stands for 60S or large ribosomal subunit while “RPS” stands for 40S or small ribosomal subunit (the 40S and 60S subunits comprise the 80S ribosomal particle which initiates and regulates translation)). Moreover, by employing the MRI-MBM CDX model, it was demonstrated that the CTC RPL/RPS gene signature was significantly expressed in CTCs from all samples analyzed either spatially or longitudinally and was significantly associated with MBM onset and progression. The discovery of enhanced expression of the CTC RPL/RPS gene signature of MBM sets the stage for the development of putative RPL/RPS therapeutic targets to improve MBM patient care.

### I. Definitions

“Patient” or “subject” as used herein means a mammalian animal, including a human, a veterinary or farm animal, a domestic animal or pet, and animals normally used for clinical research. In one embodiment, the subject of these methods and compositions is a human.

By “biomarker” or “biomarker signature” as used herein is meant a single mRNA or single protein or a combination of mRNAs and/or proteins or peptide fragments thereof, the levels or relative levels or ratios of which significantly change (either in an increased or decreased manner) from the level or relative levels present in a subject having one physical condition or disease or disease stage from that of a reference standard representative of another physical condition or disease stage. These biomarkers may be combined to form certain sets of biomarkers or ligands to biomarkers in diagnostic reagents. Biomarkers described in this specification include any physiological molecular forms, or modified physiological molecular forms, isoforms, pro-forms, and fragments thereof, unless otherwise specified. It is understood that all molecular forms useful in this context are physiological, e.g., naturally occurring in the species.

In one embodiment, at least one biomarker forms a suitable biomarker signature for use in the methods and compositions. In one embodiment, at least two biomarkers form a suitable biomarker signature for use in the methods and compositions. In another embodiment, at least three biomarkers form a suitable biomarker signature for use in the methods and compositions. In another embodiment, at least four biomarkers form a suitable biomarker signature for use in the methods and compositions. In another embodiment, at least five biomarkers form a suitable biomarker signature for use in the methods and compositions. In another embodiment, at least six biomarkers form a suitable biomarker signature for use in the methods and compositions. In another embodiment, at least seven biomarkers form a suitable biomarker signature for use in the methods and compositions. In another embodiment, at least eight biomarkers form a suitable biomarker signature for use in the methods and compositions. In still further embodiments, at least 9, at least 10, at least 11, at least 12, at least 13, at least 14, at least 15, at least 16, at least 17, or all of the biomarkers disclosed herein can be used alone or with additional biomarkers.

By “isoform” or “multiple molecular form” is meant an alternative expression product or variant of a single gene in a given species, including forms generated by alternative splicing, single nucleotide polymorphisms, alternative promoter usage, alternative translation initiation small genetic differences between alleles of the same gene, and posttranslational modifications (PTMs) of these sequences.

“Reference standard” as used herein refers to the source of the reference biomarker levels. The “reference standard” may be provided by using the same assay technique as is used for measurement of the subject's biomarker levels in the reference subject or population, to avoid any error in standardization. The reference standard is, alternatively, a numerical value, a predetermined cutpoint, a mean, an average, a numerical mean or range of numerical means, a numerical pattern, a ratio, a graphical pattern or a protein abundance profile or protein level profile derived from the same biomarker or biomarkers in a reference subject or reference population. In an embodiment, in which expression of nucleic acid sequences encoding the biomarkers is desired to be evaluated, the reference standard can be an expression level of one or more biomarkers or an expression profile.

“Reference subject” or “Reference Population” defines the source of the reference standard. In one embodiment, the reference is a human subject or a population of subjects having no melanoma, i.e., healthy controls or negative controls. In yet another embodiment, the reference is a human subject or population of subjects with one or more clinical indicators of melanoma, but who did not develop melanoma. In still another embodiment, the reference is a human subject or a population of subjects having other forms of skin cancer besides melanoma. In still another embodiment, the reference is a human subject or a population of subjects who had melanoma, following surgical removal of a tumor. In another embodiment, the reference is a human subject or a population of subjects who had melanoma and were evaluated for biomarker levels prior to surgical removal of a tumor. Similarly, in another embodiment, the reference is a human subject or a population of subjects evaluated for biomarker levels following therapeutic treatment for melanoma. In still another embodiment, the reference is a human subject or a population of subjects prior to therapeutic treatment for melanoma. In still other embodiments of methods described herein, the reference is obtained from the same test subject who provided a temporally earlier biological sample. That sample can be pre- or post-therapy or pre- or post-surgery.

Other potential reference standards are obtained from a reference that is a human subject or a population of subjects having early-stage melanoma. In another embodiment the reference is a human subject or a population of subjects having advanced stage melanoma. In still another embodiment, the reference is a human subject or a population of subjects having a subtype of melanoma.

“Sample” as used herein means any biological fluid or tissue that potentially contains melanoma biomarkers. In one embodiment, the samples may include biopsy tissue, tumor tissue, surgical tissue, circulating tumor cells, or other tissue.

Such samples may further be diluted with saline, buffer or a physiologically acceptable diluent. Alternatively, such samples are concentrated by conventional means. In certain embodiments, e.g., those in which expression levels of nucleic acid sequences encoding the biomarkers are desired to be evaluated, the samples may include biopsy tissue, surgical tissue, circulating tumor cells, or other tissue. The degree of change in biomarker level may vary with each individual and is subject to variation with each population. For example, in one embodiment, a large change, e.g., 2-3 fold increase or decrease in levels of a small number of biomarkers, e.g., from 1 to 9 characteristic biomarkers, is statistically significant. In another embodiment, a smaller relative change in 10 or more (i.e., about 10, 20, 24, 29, or 30 or more biomarkers) is statistically significant. The degree of change in any biomarker(s) expression varies with the condition, such as type or stage of melanoma and with the size or spread of the cancer. The degree of change also varies with the immune response of the individual and is subject to variation with each individual. For example, in one embodiment of this disclosure, a change at or greater than a 1.2-fold increase or decrease in level of a biomarker or more than two such biomarkers, or even 3 or more biomarkers, is statistically significant. In another embodiment, a larger change, e.g., at or greater than a 1.5-fold, greater than 1.7-fold or greater than 2.0-fold increase or a decrease in expression of a biomarker(s) is statistically significant. Still alternatively, if a single biomarker level is significantly increased in biological samples which normally do not contain measurable levels of the biomarker, such increase in a single biomarker level may alone be statistically significant. Conversely, if a single biomarker level is normally decreased or not significantly measurable in certain biological samples which normally do contain measurable levels of the biomarker, such decrease in level of a single biomarker may alone be statistically significant.

A change in level of a biomarker required for diagnosis or detection by the methods described herein refers to a biomarker whose level is increased or decreased in a subject having a condition or suffering from a disease, specifically melanoma, relative to its expression in a reference subject or reference standard. Biomarkers may also be increased or decreased in level at different stages of the same disease or condition. The levels of specific biomarkers differ between normal subjects and subjects suffering from a cancer, or between various stages of the same disease. Levels of specific biomarkers differ between pre-surgery and post-surgery patients with melanoma. Such differences in biomarker levels include both quantitative, as well as qualitative, differences in the temporal or relative level or abundance patterns among, for example, biological samples of normal and diseased subjects, or among biological samples which have undergone different disease events or disease stages. For the purpose of this disclosure, a significant change in biomarker levels when compared to a reference standard is considered to be present when there is a statistically significant (p<0.05) difference in biomarker level between the subject and reference standard or profile, or significantly different relative to a predetermined cut-point.

The term “ligand” refers, with regard to protein biomarkers, to a molecule that binds or complexes with a biomarker protein, molecular form or peptide, such as an antibody, antibody mimic or equivalent that binds to or complexes with a biomarker identified herein, a molecular form or fragment thereof. In certain embodiments, in which the biomarker expression is to be evaluated, the ligand can be a nucleotide sequence, e.g., polynucleotide or oligonucleotide, primer or probe.

As used herein, the term “antibody” refers to an intact immunoglobulin having two light and two heavy chains or fragments thereof capable of binding to a biomarker protein or a fragment of a biomarker protein. Thus, a single isolated antibody or fragment may be a monoclonal antibody, a synthetic antibody, a recombinant antibody, a chimeric antibody, a humanized antibody, or a human antibody. The term “antibody fragment” refers to less than an intact antibody structure, including, without limitation, an isolated single antibody chain, an Fv construct, a Fab construct, an Fc construct, a light chain variable or complementarity determining region (CDR) sequence, etc.

As used herein, “labels” or “reporter molecules” are chemical or biochemical moieties useful for labeling a ligand, e.g., amino acid, peptide sequence, protein, or antibody. “Labels” and “reporter molecules” include fluorescent agents, chemiluminescent agents, chromogenic agents, quenching agents, radionucleotides, enzymes, substrates, cofactors, inhibitors, radioactive isotopes, magnetic particles, and other moieties known in the art. “Labels” or “reporter molecules” are capable of generating a measurable signal and may be covalently or noncovalently joined to a ligand.

As used herein the term “cancer” refers to or describes the physiological condition in mammals that is typically characterized by unregulated cell growth. More specifically, as used herein, the term “cancer” means any melanoma. In still an alternative embodiment, the cancer is an “early stage” (I or II) melanoma. In still another embodiment, the cancer is a “late stage” (III or IV) melanoma.

The term “tumor,” as used herein, refers to all neoplastic cell growth and proliferation, whether malignant or benign, and all pre-cancerous and cancerous cells and tissues.

The term “microarray” refers to an ordered arrangement of binding/complexing array elements, e.g., nucleic acid probes or ligands, e.g., antibodies, on a substrate.

By “significant change in expression” is meant an upregulation in the expression level of a nucleic acid sequence, e.g., genes or transcript, encoding a selected biomarker, in comparison to the selected reference standard or control; a downregulation in the expression level of a nucleic acid sequence, e.g., genes or transcript, encoding a selected biomarker, in comparison to the selected reference standard or control; or a combination of a pattern or relative pattern of certain upregulated and/or down regulated biomarker genes. The degree of change in biomarker expression can vary with each individual as stated above for protein biomarkers.

The term “polynucleotide,” when used in singular or plural form, generally refers to any polyribonucleotide or polydeoxyribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The term “polynucleotide” specifically includes cDNAs. The term includes DNAs (including cDNAs) and RNAs that contain one or more modified bases. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide of less than 20 bases, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

### II. Biomarkers and Biomarker Signatures Useful in the Methods and Compositions

The “targets” of the compositions and methods of these disclosures include, in one aspect, biomarkers disclosed herein, optionally with other biomarkers identified herein, fragments, particularly unique fragments thereof, and molecular forms thereof. In certain embodiments, superior diagnostic tests for diagnosing the existence of melanoma utilize at least one of the ligands that bind or complex with one of biomarkers disclosed herein, or one of the fragments or molecular forms thereof. In other embodiments, superior diagnostic tests for distinguishing MBM utilize multiple ligands, each individually detecting a different specific target biomarker identified herein, or isoform, modified form or peptide thereof. In still other methods, no ligand is necessary.

### III. Diagnostic Reagents, Devices and Kits

### A. Labeled or Immobilized Biomarkers or Peptides or Molecular Forms

In one embodiment, diagnostic reagents or devices for use in the methods of diagnosing melanoma include one or more biomarkers disclosed herein optionally associated with a detectable label or portion of a detectable label system. In another embodiment, a diagnostic reagent includes one or more target biomarker or peptide fragment thereof identified herein, immobilized on a substrate. In still another embodiment, combinations of such labeled or immobilized biomarkers are suitable reagents and components of a diagnostic kit or device.

Any combination of labeled or immobilized biomarkers can be assembled in a diagnostic kit or device for the purposes of diagnosing melanoma, such as those combinations of biomarkers discussed herein. For these reagents, the labels may be selected from among many known diagnostic labels. Similarly, the substrates for immobilization in a device may be any of the common substrates, glass, plastic, a microarray, a microfluidics card, a chip, a bead or a chamber.

B. Labeled or Immobilized Ligands that Bind or Complex with the Biomarkers

In another embodiment, the diagnostic reagent or device includes a ligand that binds to or complexes with a biomarker disclosed herein. In one embodiment, such a ligand desirably binds to a protein biomarker, or a unique peptide contained therein, and can be an antibody which specifically binds a single biomarker disclosed herein. Various forms of antibody, e.g., polyclonal, monoclonal, recombinant, chimeric, as well as fragments and components (e.g., CDRs, single chain variable regions, etc.) or antibody mimics or equivalents may be used in place of antibodies. The ligand itself may be labeled or immobilized.

In another embodiment, suitable labeled or immobilized reagents include at least 2, 3, 4, 5, 6, 7 8, 9, 10, 11, 12, 13, 14, 15, 16, 17 or 18 or more ligands. Each ligand binds to or complexes with a single biomarker or protein/peptide, fragment, or molecular form of the biomarker(s) disclosed herein. Any combination of labeled or immobilized biomarker ligands can be assembled in a diagnostic kit or device for the purposes of diagnosing melanoma.

Thus, a kit or device can contain multiple reagents or one or more individual reagents. For example, one embodiment of a composition includes a substrate upon which the biomarkers or ligands are immobilized. In another embodiment, the kit also contains optional detectable labels, immobilization substrates, optional substrates for enzymatic labels, as well as other laboratory items.

The diagnostic reagents, devices, or kits compositions based on the biomarkers disclosed herein, optionally associated with detectable labels, can be presented in the format of a microfluidics card, a chip or chamber, a bead or a kit adapted for use with assays formats such as sandwich ELISAs, multiple protein assays, platform multiplex ELISAs, such as the BioRad Luminex platform, Mass spectrometry quantitative assays, or PCR, RT-PCR or Q PCR techniques. In one embodiment, a kit includes multiple antibodies directed to bind to one or more of the combinations of biomarkers described above, wherein the antibodies are associated with detectable labels.

In one embodiment, the reagent ligands are nucleotide sequences, the diagnostic reagent is a polynucleotide or oligonucleotide sequence that hybridizes to gene, gene fragment, gene transcript or nucleotide sequence encoding a biomarker disclosed herein or encoding a unique peptide thereof. Such a polynucleotide/oligonucleotide can be a probe or primer and may itself be labeled or immobilized. In one embodiment, ligand-hybridizing polynucleotide or oligonucleotide reagent(s) are part of a primer-probe set, and the kit comprises both primer and probe. Each said primer-probe set amplifies a different gene, gene fragment or gene expression product that encodes a different biomarker disclosed herein. For use in the compositions the PCR primers and probes may be designed based upon intron sequences present in the biomarker gene(s) to be amplified selected from the gene expression profile. The design of the primer and probe sequences is within the skill of the art once the particular gene target is selected. The particular methods selected for the primer and probe design and the particular primer and probe sequences are not limiting features of these compositions. A ready explanation of primer and probe design techniques available to those of skill in the art is summarized in U.S. Pat. No. 7,081,340, with reference to publically available tools such as DNA BLAST software, the Repeat Masker program (Baylor College of Medicine), Primer Express (Applied Biosystems); MGB assay-by-design (Applied Biosystems); Primer3 (Steve Rozen and Helen J. Skaletsky (2000) Primer3 on the WWW for general users and for biologist programmers and other publications.

In general, PCR primers and probes used in the compositions described herein are generally 17-30 bases in length, and contain about 20-80%, such as, for example, about 50-60% G+C bases. Melting temperatures of between 5° and 80° C., e.g., about 50 to 70° C. are examples.

The selection of the ligands, biomarker sequences, their length, suitable labels and substrates used in the reagents and kits are routine determinations made by one of skill in the art in view of the teachings herein of which biomarkers form signature suitable for the diagnosis of melanoma.

### IV. Methods for Diagnosing or Monitoring Melanoma

In another embodiment, a method for diagnosing or detecting or monitoring the progress of melanoma in a subject comprises, or consists of, a variety of steps.

### A. Sample Preparation

The test sample is obtained from a human subject who is to undergo the testing or treatment. The subject's sample can in one embodiment be provided before initial diagnosis, so that the method is performed to diagnose the existence of melanoma or MBM. In another embodiment, depending upon the reference standard and markers used, the method is performed to diagnose the stage of melanoma. In another embodiment, depending upon the reference standard and markers used, the method is performed to diagnose the type or subtype of melanoma. In another embodiment, the subject's sample can be provided after a diagnosis, so that the method is performed to monitor progression of a melanoma or MBM. In another embodiment, the sample can be provided prior to surgical removal of a tumor or prior to therapeutic treatment of a diagnosed melanoma and the method used to thereafter monitor the effect of the treatment or surgery, and to check for relapse. In another embodiment, the sample can be provided following surgical removal of a tumor or following therapeutic treatment of a diagnosed melanoma, and the method performed to ascertain efficacy of treatment or relapse. In yet another embodiment the sample may be obtained from the subject periodically during therapeutic treatment for a melanoma, and the method employed to track efficacy of therapy or relapse. In yet another embodiment the sample may be obtained from the subject periodically during therapeutic treatment to enable the physician to change therapies or adjust dosages. In one or more of these embodiments, the subject's own prior sample can be employed in the method as the reference standard.

Where the sample is a fluid, e.g., blood, serum or plasma, obtaining the sample involves simply withdrawing and preparing the sample in the traditional fashion for contact with the diagnostic reagent. Where the sample is a tissue or tumor sample, it may be prepared in the conventional manner for contact with the diagnostic reagent.

The method further involves contacting the sample obtained from a test subject with a diagnostic reagent as described herein under conditions that permit the reagent to bind to or complex with one or more biomarker(s) disclosed herein which may be present in the sample. This method may employ any of the suitable diagnostic reagents or kits or compositions described above.

### B. Measuring Biomarker Levels

Thereafter, a suitable assay is employed to detect or measure in the sample the p level (actual or relative) of one or more biomarker(s) disclosed herein. Alternatively, a suitable assay is employed to generate an abundance profile (actual or relative or ratios thereof) of multiple biomarkers disclosed herein from the sample or of multiple different molecular forms of the same biomarker or both.

The measurement of the biomarker(s) in the biological sample may employ any suitable ligand, e.g., nucleic acid probe, RT-PCR, antibody, antibody mimic or equivalent (or antibody to any second biomarker) to detect the biomarker. or example, the binding portion of a biomarker antibody may also be used in a diagnostic assay. As used herein, the term “antibody” may also refer, where appropriate, to a mixture of different antibodies or antibody fragments that bind to the selected biomarker. Such different antibodies may bind to different biomarkers or different portions of the same biomarker protein than the other antibodies in the mixture. Such differences in antibodies used in the assay may be reflected in the CDR sequences of the variable regions of the antibodies. Such differences may also be generated by the antibody backbone, for example, if the antibody itself is a non-human antibody containing a human CDR sequence, or a chimeric antibody or some other recombinant antibody fragment containing sequences from a non-human source. Antibodies or fragments useful in the method may be generated synthetically or recombinantly, using conventional techniques or may be isolated and purified from plasma or further manipulated to increase the binding affinity thereof. It should be understood that any antibody, antibody fragment, or mixture thereof that binds one of the biomarkers disclosed herein or a particular sequence of the selected biomarker disclosed herein may be employed in the methods described herein, regardless of how the antibody or mixture of antibodies was generated.

Similarly, the antibodies may be tagged or labeled with reagents capable of providing a detectable signal, depending upon the assay format employed. Such labels are capable, alone or in concert with other compositions or compounds, of providing a detectable signal. Where more than one antibody is employed in a diagnostic method, e.g., such as in a sandwich ELISA, the labels are desirably interactive to produce a detectable signal. In one embodiment, the label is detectable visually, e.g., colorimetrically. A variety of enzyme systems operate to reveal a colorimetric signal in an assay, e.g., glucose oxidase (which uses glucose as a substrate) releases peroxide as a product that in the presence of peroxidase and a hydrogen donor such as tetramethyl benzidine (TMB) produces an oxidized TMB that is seen as a blue color. Other examples include horseradish peroxidase (HRP) or alkaline phosphatase (AP), and hexokinase in conjunction with glucose-6-phosphate dehydrogenase that reacts with ATP, glucose, and NAD+ to yield, among other products, NADH that is detected as increased absorbance at 340 nm wavelength.

Other label systems that may be utilized in the methods and devices of this disclosure are detectable by other means, e.g., colored latex microparticles (Bangs Laboratories, Indiana) in which a dye is embedded may be used in place of enzymes to provide a visual signal indicative of the presence of the resulting selected biomarker-antibody complex in applicable assays. Still other labels include fluorescent compounds, radioactive compounds or elements. In one embodiment, an anti-biomarker antibody is associated with, or conjugated to a fluorescent detectable fluorochrome, e.g., fluorescein isothiocyanate (FITC), phycoerythrin (PE), allophycocyanin (APC), coriphosphine-O(CPO) or tandem dyes, PE-cyanin-5 (PC5), and PE-Texas Red (ECD). Commonly used fluorochromes include fluorescein isothiocyanate (FITC), phycoerythrin (PE), allophycocyanin (APC), and also include the tandem dyes, PE-cyanin-5 (PC5), PE-cyanin-7 (PC7), PE-cyanin-5.5, PE-Texas Red (ECD), rhodamine, PerCP, fluorescein isothiocyanate (FITC) and Alexa dyes. Combinations of such labels, such as Texas Red and rhodamine, FITC+PE, FITC+PECy5 and PE+PECy7, among others may be used depending upon assay method.

Detectable labels for attachment to antibodies useful in diagnostic assays and devices of this disclosure may be easily selected from among numerous compositions known and readily available to one skilled in the art of diagnostic assays. The biomarker-antibodies or fragments useful in this disclosure are not limited by the particular detectable label or label system employed. Thus, selection and/or generation of suitable biomarker antibodies with optional labels for use in this disclosure is within the skill of the art, provided with this specification, the documents incorporated herein, and the conventional teachings of immunology.

Similarly, the particular assay format used to measure the selected biomarker in a biological sample may be selected from among a wide range of protein assays, such as described in the examples below. Suitable assays include enzyme-linked immunoassays, sandwich immunoassays, homogeneous assays, immunohistochemistry formats, or other conventional assay formats. In one embodiment, a serum/plasma sandwich ELISA is employed in the method. In another embodiment, a mass spectrometry-based assay is employed. In another embodiment, an MRM assay is employed, in which antibodies are used to enrich the biomarker in a manner analogous to the capture antibody in sandwich ELISAs.

One of skill in the art may readily select from any number of conventional immunoassay formats to perform this disclosure.

Other reagents for the detection of protein in biological samples, such as peptide mimetics, synthetic chemical compounds capable of detecting the selected biomarker may be used in other assay formats for the quantitative detection of biomarker protein in biological samples, such as high-pressure liquid chromatography (HPLC), immunohistochemistry, etc.

Employing ligand binding to the biomarker proteins or multiple biomarkers forming the signature enables more precise quantitative assays, as illustrated by the multiple reaction monitoring (MRM) mass spectrometry (MS) assays. As an alternative to specific peptide-based MRM-MS assays that can distinguish specific protein isoforms and proteolytic fragments, the knowledge of specific molecular forms of biomarkers allows more accurate antibody-based assays, such as sandwich ELISA assays or their equivalent. Frequently, the isoform specificity and the protein domain specificity of immune reagents used in pre-clinical (and some clinical) diagnostic tests are not well defined. MRM-MS assays were used to quantitative the levels of a number of the low abundance biomarkers in samples, as discussed in the examples.

In one embodiment, suitable assays for use in these methods include immunoassays using antibodies or ligands to the above-identified biomarkers and biomarker signatures. In another embodiment, a suitable assay includes a multiplexed MRM based assay for two more biomarkers that include one or more of the proteins/unique peptides disclosed herein. It is anticipated that ultimately the platform most likely to be used in clinical assays will be multiplexed or parallel sandwich ELISA assays or their equivalent, primarily because this platform is the technology most commonly used to quantify blood proteins in clinical laboratories. MRM MS assays may continue to be used productively to help evaluate the isoform/molecular form specificity of any existing immunoassays or those developed in the future.

### C. Detection of a Change in Biomarker Abundance Level and Diagnosis

The level of the one or more biomarker(s) in the subject's sample or the protein abundance profile of multiple said biomarkers as detected by the use of the assays described above is then compared with the level of the same biomarker or biomarkers in a reference standard or reference profile. In one embodiment, the comparing step of the method is performed by a computer processor or computer-programmed instrument that generates numerical or graphical data useful in the appropriate diagnosis of the condition. Optionally, the comparison may be performed manually.

The detection or observation of a change in the level of a biomarker or biomarkers in the subject's sample from the same biomarker or biomarkers in the reference standard can indicate an appropriate diagnosis. An appropriate diagnosis can be identifying a risk of developing melanoma, a diagnosis of melanoma (or stage or type thereof), a diagnosis or detection of the status of progression or remission of melanoma in the subject following therapy or surgery, a determination of the need for a change in therapy or dosage of therapeutic agent. The method is thus useful for early diagnosis of disease, for monitoring response or relapse after initial diagnosis and treatment or to predict clinical outcome or determine the best clinical treatment for the subject.

In one embodiment, the change in level of each biomarker can involve an increase of a biomarker or multiple biomarkers in comparison to the specific reference standard. In one embodiment, a selection or all of the biomarkers disclosed herein are increased in a subject sample from a patient having melanoma when compared to the levels of these biomarkers from a healthy reference standard. In another embodiment, a selection or all of the biomarkers are increased in a subject sample from a patient having melanoma prior to therapy or surgery, when compared to the levels of these biomarkers from a post-surgery or post-therapy reference standard.

In another embodiment, the change in p level of each biomarker can involve a decrease of a biomarker or multiple biomarkers in comparison to the specific reference standard. In one embodiment, a selection or all of the biomarkers disclosed herein are decreased in a subject sample from a patient having melanoma following surgical removal of a tumor or following chemotherapy/radiation when compared to the levels of these biomarkers from a pre-surgery/pre-therapy melanoma reference standard or a reference standard which is a sample obtained from the same subject pre-surgery or pre-therapy. In still other embodiments, the changes in levels of the biomarkers may be altered in characteristic ways if the reference standard is a particular type of melanoma.

The results of the methods and use of the compositions described herein may be used in conjunction with clinical risk factors to help physicians make more accurate decisions about how to manage patients with melanomas. Another advantage of these methods and compositions is that diagnosis may occur earlier than with more invasive diagnostic measures.

### Exemplary Gene Transcripts and Products to Detect

In one embodiment, a product encoded by Homo sapiens ribosomal protein L12 (RPL12), mRNA NCBI Reference Sequence: NM_000976.4, e.g.,

MPPKFDPNEIKVVYLRCTGGEVGATSALAPKIGPLGLSPKKVGD
DIAKATGDWKGLRITVKLTIQNRQAQIEVVPSASALIIKALKEPPRDRK
KQKNIKHSGNITFDEIVNIARQMRHRSLARELSGTIKEILGTAQSVGCN
VDGRHPHDIIDDINSGAVECPAS

(SEQ ID NO:1), a different isoform of the protein, or a polypeptide having at least 80%, 82%, 85%, 86%, 88%, 90%, 92%, 94%, 95%, 97%, 98% or 99% amino acid sequence identity thereto,
or a gene comprising or RNA corresponding to

cctctcggct ttcggctcgg aggaggccaa ggtgcaactt ccttcggtcg tcccgaatcc
gggttcatcc gacaccagcc gcctccacca tgccgccgaa gttcgacccc aacgagatca
aagtcgtata cctgaggtgc accggaggtg aagtcggtgc cacttctgcc ctggccccca
agatcggccc cctgggtctg tctccaaaaa aagttggtga tgacattgcc aaggcaacgg
gtgactggaa gggcctgagg attacagtga aactgaccat tcagaacaga caggcccaga
ttgaggtggt gccttctgcc tctgccctga tcatcaaagc cctcaaggaa ccaccaagag
acagaaagaa acagaaaaac attaaacaca gtgggaatat cacttttgat gagattgtca
acattgctcg acagatgcgg caccgatcct tagccagaga actctctgga accattaaag
agatcctggg gactgcccag tcagtgggct gtaatgttga tggccgccat cctcatgaca
tcatcgatga catcaacagt ggtgctgtgg aatgcccagc cagttaagca caaaggaaaa
catttcaata aaggatcatt tgacaactgg tgga

(SEQ ID NO:2), a different isoform of the RNA, or a nucleic acid having at least 80%, 82%, 85%, 86%, 88%, 90%, 92%, 94%, 95%, 97%, 98% or 99% nucleic acid sequence identity thereto, is detected.

In one embodiment, a product encoded by Homo sapiens ribosomal protein L13 (RPL13), transcript variant 1, mRNA NCBI Reference Sequence: NM_000977.4, e.g.,

MAPSRNGMVLKPHFHKDWQRRVATWENQPARKIRRRKARQAKAR
RIAPRPASGPIRPIVRCPTVRYHTKVRAGRGFSLEELRVAGIHKKVAR
TIGISVDPRRRNKSTESLQANVQRLKEYRSKLILFPRKPSAPKKGDSS
AEELKLATQLTGPVMPVRNVYKKEKARVITEEEKNFKAFASLRMARAN
ARLFGIRAKRAKEAAEQDVEKKK

(SEQ ID NO:3), a different isoform of the protein, or a polypeptide having at least 80%, 82%, 85%, 86%, 88%, 90%, 92%, 94%, 95%, 97%, 98% or 99% amino acid sequence identity thereto,
or a gene comprising or RNA corresponding to

ctttccgctc ggctgttttc ctgcgcagga gccgcagggc cgtaggcagc catggcgccc
agccggaatg gcatggtctt gaagccccac ttccacaagg actggcagcg gcgcgtggcc
acgtggttca accagccggc ccgtaagatc cgcagacgta aggcccggca agccaaggcg
cgccgcatcg ccccgcgccc cgcgtcgggt cccatccggc ccatcgtgcg ctgccccacg
gttcggtacc acacgaaggt gcgcgccggc cgcggcttca gcctggagga gctcagggtg
gccggcattc acaagaaggt ggcccggacc atcggcattt ctgtggatcc gaggaggcgg
aacaagtcca cggagtccct gcaggccaac gtgcagcggc tgaaggagta ccgctccaaa
ctcatcctct tccccaggaa gccctcggcc cccaagaagg gagacagttc tgctgaagaa
ctgaaactgg ccacccagct gaccggaccg gtcatgcccg tccggaacgt ctataagaag
gagaaagctc gagtcatcac tgaggaagag aagaatttca aagccttcgc tagtctccgt
atggcccgtg ccaacgcccg gctcttcggc atacgggcaa aaagagccaa ggaagccgca
gaacaggatg ttgaaaagaa aaaataaagc cctcctgggg acttggaatc agtcggcagt
catgctgggt ctccacgtgg tgtgtttcgt gggaacaact gggcctggga tggggcttca
ctgctgtgac ttcctcctgc caggggattt ggggctttct tgaaagacag tccaagccct
ggataatgct ttactttctg tgttgaagca ctgttggttg tttggttagt gactgatgta
aaacggtttt cttgtgggga ggttacagag gctgacttca gagtggactt gtgttttttc
tttttaaaga ggcaaggttg ggctggtgct cacagctgta atcccagcac tttgaggttg
gctgggagtt caagaccagc ctggccaaca tgtcagaact actaaaaata aagaaatcag
ccatgcttgg tgctgcacac ttgtagttgc agctcctggg aggcagaggt gagggatcac
ttaacccagg aggcagaggc tgcactgagc caggatcacg ccactgcact ctagcctggg
caacagtgag actgtctcaa aaaaaaaaaa agagacaggg tcttcggcac ccaggctgga
gtacagtgcc acaatcatgg ctcactgcag tcttgaactc atggcctcaa gcagtcctcc
ctcagcctcc caagtagagg ggtttatagg cacgagaccc tgcacccaac ctagagttgc
cttttttaag caaagcagtt tctagttaat gtagcatctt ggactttggg gcgtcattct
taagcttgtt gtgcccggta accatggtcc tottgctctg attaaccctt ccttcaatgg
gcttcttcac ccagacacca aggtatgaga tggccctgcc aagtgtcggc ctctcctgtt
aaacaaaaac attctaaagc cattgttctt gcttcatgga caagaggcag ccagagagag
tgccagggtg ccctggtctg agctggcatc cccatgtctt ctgtgtccga gggcagcatg
gtttctcgtg cagtgctcag acacagcctg ccctagtcct accagctcac agcagcacct
gctctccttg gcagctatgg ccatgacaac cccagagaag cagcttcagg gaccgagtca
gattctgttt tgtctacatg cctctgccgg gtgccggtat tgaggcaccc agggagctgt
tactggcgtg gaaataggtg atgctgctac ctctgctgct gcactcacag ccacacttga
tacacgatga caccttgctt gtttggaaac atctaaacat ctagtagatg acttgcaggc
tgttggctac cagtttcctg tctgaggtgt atatgttaac ttcgtgatca gtttgtatgt
ttgggactct tgtcctatgt aaagttaagg tgggccgggt gcagtggctc acgcctgtaa
tcctaacact gggaggccga ggcgggtgga tcacctgatg gtgaaacctc atctctactg
aaaatacaaa aattagctga gtggtga

(SEQ ID NO:4), a different isoform of the RNA, or a nucleic acid having at least 80%, 82%, 85%, 86%, 88%, 90%, 92%, 94%, 95%, 97%, 98% or 99% nucleic acid sequence identity thereto, is detected.

In one embodiment, a product encoded by Homo sapiens ribosomal protein L18a (RPL18A), mRNA NCBI Reference Sequence: NM_000980.4, e.g.,

MKASGTLREYKVVGRCLPTPKCHTPPLYRMRIFAPNHVVAKSRF
WYFVSQLKKMKKSSGEIVYCGQVFEKSPLRVKNFGIWLRYDSRSGTHNM
YREYRDLTTAGAVTQCYRDMGARHRARAHSIQIMKVEEIAASKCRRPAV
KQFHDSKIKFPLPHRVLRRQHKPRFTTKRPNTFF

(SEQ ID NO:5), a different isoform of the protein, or a polypeptide having at least 80%, 82%, 85%, 86%, 88%, 90%, 92%, 94%, 95%, 97%, 98% or 99% amino acid sequence identity thereto,
or a gene comprising or RNA corresponding to

agaggacact tccttttgcg ggtggcggcg aacgcggaga gcacgccatg aaggcctcgg
gcacgctacg agagtacaag gtagtgggtc gctgcctgcc cacccccaaa tgccacacgc
cgcccctcta ccgcatgcga atctttgcgc ctaatcatgt cgtcgccaag tcccgcttct
ggtactttgt atctcagtta aagaagatga agaagtcttc aggggagatt gtctactgtg
ggcaggtgtt tgagaagtcc cccctgcggg tgaagaactt cgggatctgg ctgcgctatg
actcccggag cggcacccac aacatgtacc gggaataccg ggacctgacc accgcaggcg
ctgtcaccca gtgctaccga gacatgggtg cccggcaccg cgcccgagcc cactccattc
agatcatgaa ggtggaggag atcgcggcca gcaagtgccg ccggccggct gtcaagcagt
tccacgactc caagatcaag ttcccgctgc cccaccgggt cctgcgccgt cagcacaagc
cacgcttcac caccaagagg cccaacacct tcttctaggt gcagggccct cgtccgggtg
tgccccaaat aaactcagga acgccccggt gctc

(SEQ ID NO:6), a different isoform of the RNA, or a nucleic acid having at least 80%, 82%, 85%, 86%, 88%, 90%, 92%, 94%, 95%, 97%, 98% or 99% nucleic acid sequence identity thereto, is detected.

In one embodiment, a product encoded by Homo sapiens ribosomal protein L19 (RPL19), transcript variant 1, mRNA NCBI Reference Sequence: NM_000981.4, e.g.,

MSMLRLQKRLASSVLRCGKKKVWLDPNETNEIANANSRQQIRKL
IKDGLIIRKPVTVHSRARCRKNTLARRKGRHMGIGKRKGTANARMPEKV
TWMRRMRILRRLLRRYRESKKIDRHMYHSLYLKVKGNVFKNKRILMEHI
HKLKADKARKKLLADQAEARRSKTKEARKRREERLQAKKEEIIKTLSKE
EETKK

(SEQ ID NO:7), a different isoform of the protein, or a polypeptide having at least 80%, 82%, 85%, 86%, 88%, 90%, 92%, 94%, 95%, 97%, 98% or 99% amino acid sequence identity thereto,
or a gene comprising or RNA corresponding to

gcagataatg ggaggagccg ggcccgagcg agctctttcc tttcgctgct gcggccgcag
ccatgagtat gctcaggctt cagaagaggc tcgcctctag tgtcctccgc tgtggcaaga
agaaggtctg gttagacccc aatgagacca atgaaatcgc caatgccaac tcccgtcagc
agatccggaa gctcatcaaa gatgggctga tcatccgcaa gcctgtgacg gtccattccc
gggctcgatg ccggaaaaac accttggccc gccggaaggg caggcacatg ggcataggta
agcggaaggg tacagccaat gcccgaatgc cagagaaggt cacatggatg aggagaatga
ggattttgcg ccggctgctc agaagatacc gtgaatctaa gaagatcgat cgccacatgt
atcacagcct gtacctgaag gtgaagggga atgtgttcaa aaacaagcgg attctcatgg
aacacatcca caagctgaag gcagacaagg cccgcaagaa gctcctggct gaccaggctg
aggcccgcag gtctaagacc aaggaagcac gcaagcgccg tgaagagcgc ctccaggcca
agaaggagga gatcatcaag actttatcca aggaggaaga gaccaagaaa taaaacctcc
cactttgtct gtacatactg gcctctgtga ttacatagat cagccattaa aataaaacaa
gccttaatct gccttcc

(SEQ ID NO:8), a different isoform of the RNA, or a nucleic acid having at least 80%, 82%, 85%, 86%, 88%, 90%, 92%, 94%, 95%, 97%, 98% or 99% nucleic acid sequence identity thereto, is detected.

In one embodiment, a product encoded by Homo sapiens ribosomal protein L7 (RPL7), transcript variant 1, mRNA NCBI Reference Sequence: NM_000971.4, e.g.,

MEGVEEKKKEVPAVPETLKKKRRNFAELKIKRLRKKFAQKMLRK
ARRKLIYEKAKHYHKEYRQMYRTEIRMARMARKAGNFYVPAEPKLAFVI
RIRGINGVSPKVRKVLQLLRLRQIFNGTFVKLNKASINMLRIVEPYIAW
GYPNLKSVNELIYKRGYGKINKKRIALTDNALIARSLGKYGIICMEDLI
HEIYTVGKRFKEANNFLWPFKLSSPRGGMKKKTTHFVEGGDAGNREDQI
NRLIRRMN

(SEQ ID NO:9), a different isoform of the protein, or a polypeptide having at least 80%, 82%, 85%, 86%, 88%, 90%, 92%, 94%, 95%, 97%, 98% or 99% amino acid sequence identity thereto,
or a gene comprising or RNA corresponding to

cctctttttc cggctggaac catggagggt gtagaagaga agaagaagga ggttcctgct
gtgccagaaa cccttaagaa aaagcgaagg aatttcgcag agctgaagat caagcgcctg
agaaagaagt ttgcccaaaa gatgcttcga aaggcaagga ggaagcttat ctatgaaaaa
gcaaagcact atcacaagga atataggcag atgtacagaa ctgaaattcg aatggcgagg
atggcaagaa aagctggcaa cttctatgta cctgcagaac ccaaattggc gtttgtcatc
agaatcagag gtatcaatgg agtgagccca aaggttcgaa aggtgttgca gcttcttcgc
cttcgtcaaa tcttcaatgg aacctttgtg aagctcaaca aggcttcgat taacatgctg
aggattgtag agccatatat tgcatggggg taccccaatc tgaagtcagt aaatgaacta
atctacaagc gtggttatgg caaaatcaat aagaagcgaa ttgctttgac agataacgct
ttgattgctc gatctcttgg taaatacggc atcatctgca tggaggattt gattcatgag
atctatactg ttggaaaacg cttcaaagag gcaaataact tcctgtggcc cttcaaattg
tcttctccac gaggtggaat gaagaaaaag accacccatt ttgtagaagg tggagatgct
ggcaacaggg aggaccagat caacaggctt attagaagaa tgaactaagg tgtctaccat
gattattttt ctaagctggt tggttaataa acagtacctg ctctcaaatt gaaatatatt
gttgtatttg tgatttgttg ttgttgttgt tagcctgcct ctgtcttccc ttaactactg
tggcaagtgt ggtgtgtgat aaaattaagc caaatcaaca gcccatttcg tgcaaaatca
gggtcgagtc tgtgtgaaag acatctcttg ggtttttaaa aggcttttct atacaaaaga
ttttaatttt ttgtttttta actgtgctga gtgattcgaa atgggtttat tctaagaaag
cctgtttcac aagcatttgt acatgatttg tcggtaaggt aatccaactt tggtttatgg
aaaaaattga atttagttgc taaaatttat ttctcgggcc ttcgcttgtt tataaactgc
catgtaaatg tgttgttttc cttttgcatt cca

(SEQ ID NO:10), a different isoform of the RNA, or a nucleic acid having at least 80%, 82%, 85%, 86%, 88%, 90%, 92%, 94%, 95%, 97%, 98% or 99% nucleic acid sequence identity thereto, is detected.

In one embodiment, a product encoded by Homo sapiens ribosomal protein S12 (RPS12), mRNA NCBI Reference Sequence: NM_001016.4, e.g.,

MAEEGIAAGGVMDVNTALQEVLKTALIHDGLARGIREAAKALDK
RQAHLCVLASNCDEPMYVKLVEALCAEHQINLIKVDDNKKLGEWVGLCK
IDREGKPRKVVGCSCVVVKDYGKESQAKDVIEEYFKCKK

(SEQ ID NO:11), a different isoform of the protein, or a polypeptide having at least 80%, 82%, 85%, 86%, 88%, 90%, 92%, 94%, 95%, 97%, 98% or 99% amino acid sequence identity thereto,
or a gene comprising or RNA corresponding to

ctctttccct gccgccgccg agtcgcgcgg aggcggaggc ttgggtgcgt tcaagattca
acttcacccg taacccaccg ccatggccga ggaaggcatt gctgctggag gtgtaatgga
cgttaatact gotttacaag aggttctgaa gactgccctc atccacgatg gcctagcacg
tggaattcgc gaagctgcca aagccttaga caagcgccaa gcccatcttt gtgtgcttgc
atccaactgt gatgagccta tgtatgtcaa gttggtggag gccctttgtg ctgaacacca
aagaaataaa tctttggctc aca gaaactagga gaatgggtag gcctttgtaa
aatcaaccta attaaggttg atgacaacaa ggttggttgc agttgtgtag tagttaagga
aattgacaga gaggggaaac cccgtaaagt cattgaagag tatttcaaat gcaagaaatg
ctatggcaag gagtctcagg ccaaggatgt

(SEQ ID NO:12), a different isoform of the RNA, or a nucleic acid having at least 80%, 82%, 85%, 86%, 88%, 90%, 92%, 94%, 95%, 97%, 98% or 99% nucleic acid sequence identity thereto, is detected.

In one embodiment, a product encoded by Homo sapiens ribosomal protein S18 (RPS18), mRNA NCBI Reference Sequence: NM_022551.3, e.g.,

MSLVIPEKFQHILRVLNTNIDGRRKIAFAITAIKGVGRRYAHVV
LRKADIDLTKRAGELTEDEVERVITIMQNPRQYKIPDWFLNRQKDVKDG
KYSQVLANGLDNKLREDLERLKKIRAHRGLRHFWGLRVRGQHTKTTGRR
GRTVGVSKKK

(SEQ ID NO:13), a different isoform of the protein, or a polypeptide having at least 80%, 82%, 85%, 86%, 88%, 90%, 92%, 94%, 95%, 97%, 98% or 99% amino acid sequence identity thereto,
or a gene comprising or RNA corresponding to

ctctcttcca caggaggcct acacgccgcc gcttgtgctg cagccatgtc tctagtgatc
cctgaaaagt tccagcatat tttgcgagta ctcaacacca acatcgatgg gcggcggaaa
atagcctttg ccatcactgc cattaagggt gtgggccgaa gatatgctca tgtggtgttg
aggaaagcag acattgacct caccaagagg gcgggagaac tcactgagga tgaggtggaa
cgtgtgatca ccattatgca gaatccacgc cagtacaaga tcccagactg gttcttgaac
agacagaagg atgtaaagga tggaaaatac agccaggtcc tagccaatgg tctggacaac
aagctccgtg aagacctgga gcgactgaag aagattcggg cccatagagg gctgcgtcac
ttctggggcc ttcgtgtccg aggccagcac accaagacca ctggccgccg tggccgcacc
gtgggtgtgt ccaagaagaa ataagtctgt aggccttgtc tgttaataaa tagtttatat
acctatggc

(SEQ ID NO:14), a different isoform of the RNA, or a nucleic acid having at least 80%, 82%, 85%, 86%, 88%, 90%, 92%, 94%, 95%, 97%, 98% or 99% nucleic acid sequence identity thereto, is detected.

In one embodiment, a product encoded by Homo sapiens ribosomal protein S24 (RPS24), transcript variant a, mRNA NCBI Reference Sequence: NM_033022.4, e.g.,

MNDTVTIRTRKFMTNRLLQRKQMVIDVLHPGKATVPKTEIREKL
AKMYKTTPDVIFVFGFRTHFGGGKTTGFGMIYDSLDYAKKNEPKHRLAR
HGLYEKKKTSRKQRKERKNRMKKVRGTAKANVGAGKK

(SEQ ID NO:15), a different isoform of the protein, a or a polypeptide having at least 80%, 82%, 85%, 86%, 88%, 90%, 92%, 94%, 95%, 97%, 98% or 99% amino acid sequence identity thereto,
or a gene comprising or RNA corresponding to

ctcttttcct ccttggctgt ctgaagatag atcgccatca tgaacgacac cgtaactatc
cgcactagaa agttcatgac caaccgacta cttcagagga aacaaatggt cattgatgtc
cttcaccccg ggaaggcgac agtgcctaag acagaaattc gggaaaaact agccaaaatg
tacaagacca caccggatgt catctttgta tttggattca gaactcattt tggtggtggc
aagacaactg gctttggcat gatttatgat tccctggatt atgcaaagaa aaatgaaccc
aaacatagac ttgcaagaca tggcctgtat gagaagaaaa agacctcaag aaagcaacga
aaggaacgca agaacagaat gaagaaagtc agggggactg caaaggccaa tgttggtgct
ggcaaaaagt gagctggaga ttggatcaca gccgaaggag taaaggtgct gcaatgatgt
tagctgtggc cactgtggat ttttcgcaag aacattaata aactaaaaac ttca

(SEQ ID NO:16), a different isoform of the RNA, or a nucleic acid having at least 80%, 82%, 85%, 86%, 88%, 90%, 92%, 94%, 95%, 97%, 98% or 99% nucleic acid sequence identity thereto, is detected.

In one embodiment, a product encoded by Homo sapiens ribosomal protein S26 (RPS26), rRNA NCBI Reference Sequence NM_001029.5, e.g.,

MTKKRRNNGRAKKGRGHVQPIRCTNCARCVPKDKAIKKFVIRNI
VEAAAVRDISEASVFDAYVLPKLYVKLHYCVSCAIHSKVVRNRSREARK
DRTPPPRFRPAGAAPRPPPKPM

(SEQ ID NO:17), a different isoform of the protein, or a polypeptide having at least 80%, 82%, 85%, 86%, 88%, 90%, 92%, 94%, 95%, 97%, 98% or 99% amino acid sequence identity thereto, or a gene comprising or RNA corresponding to

gattttcttc cgccatccgg ctaaatagtc ccatgtgcac tttgttccat ggataaataa
acactaggaa cgcatttcca ccctagattt cagcagaaat gctgaatgta aaggaatatt
tgagtaaagt gagttgccgt tcttgaagcc cgtctcctaa ggattctccc ggtgtccgcg
tagggatctc atgctatata ggagggccct gccaggcacc gtctcctctc tccggtccgt
gcctccaaga tgacaaagaa aagaaggaac aatggtcgtg ccaaaaaggg ccgcggccac
gtgcagccta ttcgctgcac taactgtgcc cgatgcgtgc ccaaggacaa ggccattaag
aaattcgtca ttcgaaacat agtggaggcc gcagcagtca gggacatttc tgaagcgagc
gtcttcgatg cctatgtgct tcccaagctg tatgtgaagc tacattactg tgtgagttgt
gcaattcaca gcaaagtagt caggaatcga tctcgtgaag cccgcaagga ccgaacaccc
ccaccccgat ttagacctgc gggtgctgcc ccacgtcccc caccaaagcc catgtaagga
gctgagttct taaagactga agacaggcta ttctctggag aaaaataaaa tggaaattgt
acttaatatt gcatgttaag tgtatctgtg ccagataagg tggggatttt gtgtgttaga
ccaagtgtga agtgacacac attattttca tggggaagaa agcttattca tgtaatttaa
tttttttctt tttttttttt ttttttttga gacggagtct ttgtcgccca agctgaattg
cagtggcgtg atctcagctc actgcaacct ccgtctcccg ggttcaagtg attctcctgc
ctcagcttct tgagtagctg ggattacagg tgtctgccac catgcctggt taatttttgt
atttttggta gagatggggt ttcactatgt tgtccaggct ggtcttgaac ttctgacctc
agttaatcca ccagccttgg cctcccaaag tgctgggatt acaggcttga gccacctcgc
ctagctattt atgtaaatta aactttaatt gtggtcgtat ggttggcctc acaattcgca

(SEQ ID NO:18), a different isoform of the RNA, or a nucleic acid having at least 80%, 82%, 85%, 86%, 88%, 90%, 92%, 94%, 95%, 97%, 98% or 99% nucleic acid sequence identity thereto, is detected.

In one embodiment, a product encoded by Homo sapiens ribosomal protein L23 (RPL23), mRNA NCBI Reference Sequence: NM_000978.4, e.g.,

MSKRGRGGSSGAKFRISLGLPVGAVINCADNTGAKNLYIISVKG
IKGRLNRLPAAGVGDMVMATVKKGKPELRKKVHPAVVIRQRKSYRRKDGVFLYFEDNA
GVIVNNKGEMKGSAITGPVAKECADLWPRIASNAGSIA

(SEQ ID NO:19), a different isoform of the protein, or a polypeptide having at least 80%, 82%, 85%, 86%, 88%, 90%, 92%, 94%, 95%, 97%, 98% or 99% amino acid sequence identity thereto,
or a gene comprising or RNA corresponding to

cggcgttcaa gatgtcgaag cgaggacgtg gtgggtcctc tggtgcgaaa ttccggattt
ccttgggtct tccggtagga gctgtaatca attgtgctga caacacagga gccaaaaacc
tgtatatcat ctccgtgaag gggatcaagg gacggctgaa cagacttccc gctgctggtg
tgggtgacat ggtgatggcc acagtcaaga aaggcaaacc agagctcaga aaaaaggtac
atccagcagt ggtcattcga caacgaaagt cataccgtag aaaagatggc gtgtttcttt
attttgaaga taatgcagga gtcatagtga acaataaagg cgagatgaaa ggttctgcca
ttacaggacc agtagcaaag gagtgtgcag acttgtggcc ccggattgca tccaatgctg
gcagcattgc atgattctcc agtatatttg taaaaaataa aaaaaaaaac taaacccatt
aaaaagtatt tgtttgcagt gcttgtctcc ctgttcactt ccagggttca agcgattctc
ctgcctcagc ctcctaagta gctgggatta caggcacctg ccgtcatgcc tggctagttt
ttgtattttt ggtggagaca gggtttcacc atgtttgcca aacgactcaa actcctgatc
tcaagtgatc tgcccaccca ggcctctaca gtgtttttga cataccctga ccatcacttt
tctgaaatgg aaactctggg catttttttt ttaaagcaat ccttgctttt ttgtgagttt
gcagactttc agcatcttcc aattgctgta tttacaattt tggcctcaaa aagtattatt
tgggttttga gtcccaaaaa taatagtaga tactgctcaa tgactggcta tacatggtag
cttctcctgg agtgaggaag gcattcaaat ggctgggcgc ggtagctggc acctgtaatc
ccaacacttt cggaggccaa ggcaggcaga tcatgaggtc aggagttcaa gaccaggctg
accaacgtgg tgaaacctcc tcttttctaa aaatacaaaa attagctggg cgtggtggcg
caccgcctgt aatctcagct actgagagaa tcgcttgaac ctgggaggtg gagtttgcag
cgagtcgaga ttgcaccatt gtactccatc cagcctgggc aacggagcaa gattccgttc
ccgcccccgc cccaaaaaaa ggcattgaag ttaagataga ctatatagtt agatcctgga
cacacacatg gattttgagg aatgatgtga gtttgtttat gtatgtacat tttagcagtt
aacagatttg gagtaaattg aatatttata aaacaacagt aattgccatg taggtttact
gtcatagtgg aagatgatta agttgatggt acctacaggt attttgctat gaaaatgttt
tgacaaacag gatgatcgtg taatttatgt tccaagctct agtttgagaa tggaagaatg
tggtaaattt ttgccaactg aacaggcata aagcgctgat aataagggac ttggccttaa
ggtaggaggt tgttagcatt tctttctaaa cgtgtaagag tttatttagg tgacacccag
cgttttggaa aaatgggtgt ttgtttagaa caataatttg gagggaagtg gactaaacag
ggtttttaga ttaaggtttg tgtttatgta tctgtatctg caaatactca gccataaatg
tttctacctg taagttgggt ataatacaag ctccacttgg tatcaaaaag gactaccctt
agtgtcttcc atgactagtt atggaatgta ccagacctag agaggagttg ttctaacctg
gagcttttga aaatgtttcc ggtccatacc ctagaccaat taagtcagac tgcagaatag
gactcagaca tcagaattgt gaagctccca ggagatgtca aggtataccc aagactgaga
accagtaccc tgtctagact gaaccaggct tggttttaga agtattaaat ctcgcctggg
tacggtggct cacacctata atcccagcac tttgggaggc tgaggtgggt ggatcatctg
aggtcaggag agtgagacca gcctgtccaa catgttgaaa ccccatctct actatacaaa
attagccagg catggtggtg catgctgtaa tcccagctac ttgggaggct ttgtgctcca
aattgcttga accccggaga tggaggttgc agtgagctga ggtcgcacga gaggcaggag
gcctgggcaa ccagtgaaac tctgggggga aagaaaaaaa atgtattaaa tctctagttt
tagagagtta caccataaca tccctgagta tggtcaattt caagtcagct ttagtgtgac
aagcccctag gcccaccatt tatgtcttta tatattatgg caatatatga tccttatata
ttaaccacta atcgctgctg tttttgaatg ttatcttttc tgaggcagtc ttgctctgtc
acacaggctg gagtgcagta acaccactga agctcactgt aactttgaac tgctggactt
ggaatcctcc tgctgtggcc ttcaaagggc tgcaattaca agtgtgagcc actgcatccc
acctcacatt ttattctttg gagatttttt ttgacttgga ttaaaaaact ttatatttac
acttca

(SEQ ID NO:20), a different isoform of the RNA, or a nucleic acid having at least 80%, 82%, 85%, 86%, 88%, 90%, 92%, 94%, 95%, 97%, 98% or 99% nucleic acid sequence identity thereto, is detected.

In one embodiment, a product encoded by Homo sapiens ribosomal protein L26 (RPL26), mRNA NCBI Reference Sequence: NM_000987.5, e.g.,

MKFNPFVTSDRSKNRKRHFNAPSHIRRKIMSSPLSKELRQKYNV
RSMPIRKDDEVQVVRGHYKGQQIGKVVQVYRKKYVIYIERVQREKANGTTVHVGIHPS
KVVITRLKLDKDRKKILERKAKSRQVGKEKGKYKEETIEKMQE

(SEQ ID NO:21), a different isoform of the protein, or a polypeptide having at least 80%, 82%, 85%, 86%, 88%, 90%, 92%, 94%, 95%, 97%, 98% or 99% amino acid sequence identity thereto,
or a gene comprising or RNA corresponding to

ctcttccctt ttgcggccat caccgaagcg ggagcggcca aaatgaagtt taatcccttt
gtgacttccg accgaagcaa gaatcgcaaa aggcatttca atgcaccttc ccacattcga
aggaagatta tgtcttcccc tctttccaaa gagctgagac agaagtacaa cgtgcgatcc
atgcccatcc gaaaggatga tgaagttcag gttgtacgtg gacactataa aggtcagcaa
attggcaaag tagtccaggt ttacaggaag aaatatgtta tctacattga acgggtgcag
cgggaaaagg ctaatggcac aactgtccac gtaggcattc accccagcaa ggtggttatc
agtaatctta tatacaagct ttgattaaaa cttgaaacaa agagcctg

(SEQ ID NO:22), a different isoform of the RNA, or a nucleic acid having at least 80%, 82%, 85%, 86%, 88%, 90%, 92%, 94%, 95%, 97%, 98% or 99% nucleic acid sequence identity thereto, is detected.

In one embodiment, a product encoded by Homo sapiens ribosomal protein L35a (RPL35A), mRNA NCBI Reference Sequence: NM_00996.4, e.g.,

MSGRLWSKAIFAGYKRGLRNQREHTALLKIEGVYARDETEFYLG
KRCAYVYKAKNNTVTPGGKPNKTRVIWGKVTRAHGNSGMVRAKFRSNLPAKAIGHRIR
VMLYPSRI

(SEQ ID NO:23), a different isoform of the protein, or a polypeptide having at least 80%, 82%, 85%, 86%, 88%, 90%, 92%, 94%, 95%, 97%, 98% or 99% amino acid sequence identity thereto,
or a gene comprising or RNA corresponding to

cttctcttac cgccatcttg gctcctgtgg aggcctgctg ggaacgggac ttctaaaagg
aactatgtct ggaaggctgt ggtccaaggc catttttgct ggctataagc ggggtctccg
gaaccaaagg gagcacacag ctcttcttaa aattgaaggt gtttacgccc gagatgaaac
agaattctat ttgggcaaga gatgcgctta tgtatataaa gcaaagaaca acacagtcac
tcctggcggc aaaccaaaca aaaccagagt catctgggga aaagtaactc gggcccatgg
aaacagtggc atggttcgtg ccaaattccg aagcaatctt cctgctaagg ccattggaca
cagaatccga gtgatgctgt acccctcaag gatttaaact aacgaaaaat caataaataa
atgtggattt gtgctcttgt atttttaagt ggattaaaaa acttactacc ttaaattgat
ttgctacatg cttaaaatga tagaggttgc tcagcatttt tggagtacaa gggggtcaga
gagacatgtg atgaaaatta cagggcgagt acagagattt agaagggaac gggttttaat
gcgagtatct ttgacagagt cttgctctgt tgcccatgct ggagtgtagt ggtgctcgct
gcagcctcac attcaaaggc tcaagcaatc ctcccttggc ctttgaagta gctgggacca
caggctcatg ccaccatccc tgggtcattt ttaaattttt tgtagagagg gtctgactct
tgcctatgct ggcttcaaac tcctgggctc aagcaatcct ccttccttgg cctctcctga
agtgctggga tacagttatg agccaccaca cctgccaagt gctttgtgat actatgcatt
tgttcaatgc agattgggaa acttaaaatt tgaatggaga ttatgttgat gggctttggc
agttcatttg gatagactgg gatgagaagc tcttgggact tgtgactgga caaagcattc
cagtatatta aaataaaatt aagccatatt actccactca taaaaagcaa tcctatggta
ggtacatgga ggttgggaat agtgcacgga aaggtggcag ctttctttgg cttcatgttt
taatctggta aagttcaaga ttgcacttta agcaggcctc ctaaatattt tagatttctt
ggggatatgc taaaataaaa caactaaggc atca

(SEQ ID NO:24), a different isoform of the RNA, or a nucleic acid having at least 80%, 82%, 85%, 86%, 88%, 90%, 92%, 94%, 95%, 97%, 98% or 99% nucleic acid sequence identity thereto, is detected.

In one embodiment, a product encoded by Homo sapiens ribosomal protein L37 (RPL37), mRNA NCBI Reference Sequence: NM_000997.5, e.g.,

MTKGTSSFGKRRNKTHTLCRRCGSKAYHLQKSTCGKCGYPAKRK
RKYNWSAKAKRRNTTGTGRMRHLKIVYRRFRHGFREGTTPKPKRAAVAA
SSSS

(SEQ ID NO:25), a different isoform of the protein, or a polypeptide having at least 80%, 82%, 85%, 86%, 88%, 90%, 92%, 94%, 95%, 97%, 98% or 99% amino acid sequence identity thereto,
or a gene comprising or RNA corresponding to

ctcttccggt ctttctggtc tcggccgcag aagcgagatg acgaagggaa cgtcatcgtt
tggaaagcgt cgcaataaga cgcacacgtt gtgccgccgc tgtggctcta aggcctacca
ccttcagaag tcgacctgtg gcaaatgtgg ctaccctgcc aagcgcaaga gaaagtataa
ctggagtgcc aaggctaaaa gacgaaatac caccggaact ggtcgaatga ggcacctaaa
aattgtatac cgcagattca ggcatggatt ccgtgaagga acaacaccta aacccaagag
ggcagctgtt gcagcatcca gttcatctta agaatgtcaa cgattagtca tgcaataaat
gttctggttt taaaaaatac atatctggtt ttggtaaggt atttttaatc aattaggctt
gtagtatcag tgaaatactg taggtttagg gactgggcta gcttcatatc agatttactt
gttaagtgac tgttttggaa tgtttacttt tggactgggt ttgtaacacg gttaaaggca
atgagaaaca agcagaattc caggagtcct tgaagcagag ggcactggaa gacaatatag
cagattaaaa tagcacagct catgtggcat aggtgggtat tttagatgtt tgagtaaatt
tgaaagagta tgatgtttaa attaccttta gcaacatgtt catctgctat gctgtcatga
ctagggggat gattattagt cacatagagc ttgggagtac cactggaaac gtatgggtag
gagtttaggt ggcttctgtt tttcaaaaga tgatcttatc ctagtatctg taatgctcac
ttggcacacc tgacttgtgg gctgtgtgta aggtggctag ctaagtgaaa aaagcctgct
aggtgtgagt caacttaaga atatgtaaat aggtttgaga aaaagtaggg cttgggtgca
agtaaagatt gagcaggaaa taaaggaaaa tcaagtataa tccctgagat ttgtagacta
aaggcaatga tgtgggacta cttggtcgaa tttttttagc cctcaacttg gtaattgggt
gtttctgtgt taaagcactg aaacttgctg tcgtgccttc ctagttttcg tggtttattg
acagggttgg gggttttttt tgttttttta aaatgaaggg acaaagtcaa ctggactgct
gagtgagagg gcaggggcag ttgaagggaa catgaattgc tggaacagct acataaaata
gtgatgtagc caagtcatgc tatttaaatt ataattctcc actgtgttta gaataacatc
tgaggttctt aacctggcct tggaagggta tcacttttac ttgtaacctg gaatggcttt
ataatgtgct agctaattgc tactctcatc ttgtatttta actcctaatt tacccttcag
gtctcagctt cagaacattc acttataaag aaaccctgct gattaaatct ctcttgggct
tcctcccgaa atgtgagact atactttaaa gatgtatggt tagagtccaa ttgccattgc
ctttcttgtt tacagatagt gcaatggcgc aatcctggct cactgcagcc tctaattcct
gggctcaagt caagtggttc tcctgcctca gccttctgag tagctgggac tacagatgca
caccaccaca cctggctaat ttttgtgttt tttgtagaga tggagattca ctcgactaat
tctttttgta ttcttagtag agactgggtt tcaacatgtt gaccaggctg gtctcgaact
cgacctcgtg atccgccgcc tcggcctccc aaagtgctag gattacaggc gtgagccacc
actcccggct gtcatcatca aattttcaag tgaagatagt ctgttgaaga ttgaacaatg
accttgaaag acagctgagt tgctgtgggt ataatgtaaa gctggtgaag tttggccagt
ttgggcttca gaagtctaag tctagtgaag gtaccctgac ccccatataa acaacccttg
aggccggtgt ggtggctcac ggctgtaatc ctggcacttt gggaggccga ggtgggcgga
tcacaaggtc aagagatcga gatcaccctg gcccacatgg tgagacccca actctactaa
aaatacaaaa attagctggg tgtggtggca tgtgcctgta gtcttagcta cttgagaggc
tgaggcagga gaatcgcttg aacctgggag gcagaggttg cagtgagctg agatcatgcc
actgtgctcc agcctggtgg cacagtgaga ctccacctca aaaaaaaaaa aaaatccttg
aactaatgac tcaaattttc aaatgaaaca aaataagcag tggatcttgc attggagatg
gagttaaact atgttgccca ggctggtctt ggactcctgg tttcaagtga tcctctcatc
ttggcctccc aaagtgctgg gattacaggg acgagccacc acaccccacc tattgtctat
atttctatct ttaacagcac ttcagtcctg ccttaagtta tagttatgta tagataccca
ttatacttta aatttttcag cagaaattat gcttttatct tctctgcagt gttatatgtt
ggtgtgcaaa aatgttaaat ttatttttcc taagtacccc atctgctttt caactctgtc
ctctgcctga aaagcctccc tccagcccct acttccctcc catcttagtt cacaaagtca
ggttgatttg cccccagctg tcaaagcaga ctacctgttt ccacatgtaa ctggttatgt
tctgtaaagt tacaaaaata gaaaggttga atctgtggcg gccgggtacg gtagctcacg
tcctatagtc gcagcacttt gggaggccaa ggtgggcagt tcacgtgagg tggggagttc
gagaccagcc tgaccaacat ggagaaaccc cgtctctact aaaaatacaa aaatattagc
cgggcgtggt ggtgcatgcc tgtaatccca gctactaggg aggctgaggc aggagaattg
cttgaacctg ggagacggag gttgcggtga gctgcagagg cctcacctct aattgagaca
caattatata ttgttgatat atatatatat atatatacac acacacacac acactatgat
ggataaatgc atgagtttct gtgagagcat tggaaaggag tttgtcactc aataggtgaa
gccaggctaa gatttaagct gagccaggga ggacttgaag gaatcatgat gagagagaag
gtaagtggct ttgccagcaa tgaaacagct gacataatgg taaccagtca gaggagggca
taactatgaa actggacacc ttggttgtca ggttagaagg atggggtgta gggttggtaa
gaaaagaatt cagggaagag cagcgatcag attatgaaga atttgtcttg agaaattaca
gaggatttaa accagaatgt taggaatagt tattctagca agatgaatgt ggaaagtgtt
agtgtgcatg tgatgagtct tgaagctgga aactaggtaa caggttctta aatagttcat
gtgaaaatca tgacagacta aggcagtggc tgtggggctg tccgggagtt ctctacagaa
aacatctaaa acttgaatgt gcaagtgagt agctaacttc caagcttccc atttctgtat
aatttaagca tgaaaatgag aacactgaga tttgataggc atgtagaagt cagagtaagc
aagagggctt gagttcatcg atgaacttca gtaactatcc ttgacttagt attggtggaa
accatttgtg aatttacaga ctccaaaaac aaaaagataa tttagagtct caaagtaaga
tttggggaga tgtccattgg agacagaagt ggagagaggg agaagttcat ttgttgacat
atttatgtat ggtgcaccta ctgtgtgcca agccatgtta gagatacagt gggagcaaaa
ccagatgtgt tttctgccct ctgactccag tggttgcaga attagcccag gaactagaat
tggatggagt cacacaaacc aaggaagggg attgtttgag gggggcactc agtcacatgt
ggctgaggcc aaagagaatg aagatatatt ttaaaattct ggatttggca aaatttaggc
tagtctttac ttttcataaa cctctctcat gttagagcag caaagaaaaa ccagtttgcc
aagaggtcac gagattgggc agtgaggaat ggggagttta gtttaaaaga caacactaga
tacttttgga agtttaccta ttctgtggtt cccttttatt gactaaatgg tgcccttcat
tcatgaagca agtatctatt tagtgactac tatgtgtcaa aagctatcta ggcccagaaa
cttttaagtg gaaaacaaaa caaagaccct gccctgacag atttcaatca tgtatatact
gtatgtatgt ttgtggaagg tgacataata gacaattgca gatagtgata agtgtgtaag
acaaggatac cacgagcgta atagacagtg gggatggagg aacctttaga aggttggtta
agttgaatga taaggagcca ttcttttgtc tttacctgaa tgacttatca ttcagccctt
tttgagtttt gggttgcttg cagagtttaa ccttgcctgt aattgaaact taattttgta
atagcataac ttcatgtaag aaaagcaaag caatacatta cagaattatt ttactgaaga
acttgtttca gagaaagagg ctgtttcaca tttattagca catttggatt atatttaggt
gttttatttt tttttaaaca aaggagtttg gatcataata caagagaagc acagggcaaa
gacactgcat aacctcaaga actaagaatg gaaggactgg ccaggcctgg tggtgcacac
ctgtaatccc agctactcag gaggctgagg cgagagaatc gcttgagcct ggggggcaga
ggttgcagtg agctgagaga acggcgggcg gcagagcgag actccatctc aaaaaaaaaa
aaggataaag gaaggactta agcaaaatct tccttgtaag tagaaggatg ttttgacaag
aaaagttgca atggaaaaat ggttctcatg tacacgagta tgtagaataa gcatcgtgtg
tggattggat tcagatcaaa acattgcttt tatgtttgtg tctttatacg gtgggagtat
accctggtgc cccaggatga agacttgacc tgacccatgt atttttagat tactcacaga
taacaaaaag tattttcatc atgattagtt gcgaaaacag ttttatttca ataggtaaaa
cgtgcagtcc tatgtaatcg tcagaaggta atcttaatta tagcttgggt gtgctttaaa
ctgcaagctg gcagtggagg gcacgattcc tctgatttca gctttctcct tatacttttc
tggagctgtg agctgcaagt taactcagtg ggattaaagt gtagactgga ggtacaaaag
gtgaggagtg aggagatagg gtagttcttc cttggctggc tggcttcata atccctgggc
cccgcagata attaaatcga ctttttctgt ctcaggcatt tgtatgacct ctttggaggt
tccctgctgg gtagttatcc ttgtatctga tggacccatc tcaatttaaa atactctgcc
aggttcggag gttcatgctt gtcatcccag cactttggga ggctcagagg tgccattggc
ttgagcccaa gagtttgaga ccagcctggg caacctggtg aaacctcttc tccattaaaa
atacaaaaaa ttagccaggc atggtggtat gtgcctgtag tcccagctac tcagggagct
gaggtggaag gattgcttga gcctgggagg tcaaggctgc agtgaacctt gattgtgcca
ctgcactcca gcctggatga caaagaccct gtctcaagaa aagtctgtaa ttctttccta
acccttagta tccagcctca gtcctgaggt tttctttacc tctgggggct attttatgcc
ggctctctcc tgagtgtcac acatctggtc ctcaggaaac attctcacat ccctggcctg
aaaaaaacaa tttcagggag atcgcatggc agcagccctc tctgggctcc ccagctaaat
ggttgtacga aacacatttc aaagctctct gaagggcttc cttgaagttc ctttcactgg
gtttcaaagt agaaggtagt aactcctttg tccaagaagg ctgaattgag cacttaacaa
ttctccaaag aaatttcttc attggtctct atcctagacc ccttctgtat ccttgatatg
gctgaggatt ctaaaaaatg accagtctta catgggaggg ctgggatata aaaaataaaa
taataaataa tgataaaaac tgaaaactga ccacgttctt ggatatgttt tctttgggtt
gtgtgtgtgt gtgagacttt tgatagttac ccaaagtagg aaaaatccca ttctaataag
gttatattta tgtagctctg caaataaaca tctagcaaat gtaaaaagta ttttctttgc
cttaaaaatg attaaaatta tttgaactcc tgaggagtgt tatatgaata aaattagtaa
gttatttgga ggaaagttat tttttaaaaa gacaactggt aaaacagtac aggagaaagg
ccagcttcct caagtgagga cagttgttta gaattgactg aggagcggcc gggtgcggag
gctcacatct gtaatcccac acgccttggg aggctctgag gcgggtagat cacctgaggt
caggagtttg agaccagcct ggccaacatg gcgaaacccc gtctccacta aaaatacaaa
aattagccag gtgcagtggc acacacctgt aatcccagct actccgaagg ctgaggcagg
aggatcacct gagcccagga agttgagact gcagtgagct gagattgcac cactgcactc
cagcctcagt gacagcgaga ctgtctcaaa aaaagaaaaa agtgactgag gaggaagagg
ccaggtggca aatggaacag aatcaccaaa gggtgaacag gactaaggca atgtagtgta
tggctcagct acgtcagagt ggaaaaggtg ttattagagc agaaactatg gtccctgcgt
cacagggaag caacctacag agaagcagca gctccccaag agaggagaga taagaagcca
gaagcctcag agtgaacaat tgtcctatta gggattgctc tagagagaga aacctctggg
aacgtacccc tgtgaggcag cacagcacaa tgcttttaga attgtatgag agttgatata
tctccatttg ttttgcaaag gcaaaaacta agatacagag attatctgtt aaagttatgt
atttctttgg taataaagat gctgacagtg ttgctggaat gcatttcttt aataaagata
ttgtacaatg aaa

(SEQ ID NO:26), a different isoform of the RNA, or a nucleic acid having at least 80%, 82%, 85%, 86%, 88%, 90%, 92%, 94%, 95%, 97%, 98% or 99% nucleic acid sequence identity thereto, is detected.

In one embodiment, a product encoded by Homo sapiens ribosomal protein L38 (RPL38), mRNA NCBI Reference Sequence: NM_000999.4, e.g.,

MPRKIEEIKDELLTARRKDAKSVKIKKNKDNVKFKVRCSRYLYT
LVITDKEKAEKLKQSLPPGLAVKELK

(SEQ ID NO:27), a different isoform of the protein, or a polypeptide having at least 80%, 82%, 85%, 86%, 88%, 90%, 92%, 94%, 95%, 97%, 98% or 99% amino acid sequence identity thereto,
or a gene comprising or RNA corresponding to

ctttttcgtc cttttccccg gttgctgctt gctgtgagtg tctctagggt gatacgtggg
tgagaaaggt cctggtccgc gccagagccc agcgcgcctc gtcgccatgc ctcggaaaat
tgaggaaatc aaggacttcc tgctcacagc ccgacgaaag gatgccaaat ctgtcaagat
caagaaaaat aaggacaacg tgaagtttaa agttcgatgc agcagatacc tttacaccct
ggtcatcact gacaaagaga aggcagagaa actgaagcag tccctgcccc ccggtttggc
agtgaaggaa ctgaaatgaa ccagacacac tgattggaac tgtattatat taaaatacta
aaaatcctaa gtgtctttcg tctttgcgga tgggaaaggg aaaaatgcta cctcgtagtg
gcttctgatg ggaacaggac gcgggttctg ttgctgcctt cctgtgtctt tttttttttt
tttttttctt tctttgagac ggagtcttgc tctgtggctc atcctggagc acagtggtgc
gatatcagct cactaccacc tccgcctcct gggttcaagc gactgtcctg cctcagcctc
ccgagtggct gggattacag gcacacatca ccacgcctgg ccaatttttg tatttgtagt
agagacaggg tttcactgcc tgcctcagcc tcccatagtg ctgggattac aggcatgagc
ctccgtgccc ggtgcatccc taatcttgag catgatctca gtcggcaaat gaggccatct
gttttcagcc tgtttgaaaa taagatgtgg ggaggccatg atggaaatag cacgtggggt
taaacataac tggcagatgt gggagcgatg gtggggcatg ccattcaaac aggtcccaaa
atgggtgcaa caaggtatag cacatctacc actcgctaac ttgactgact tggagaaatg
actacacttt tgcctgtttc ctcagttgga aaatagccat attaacacct ctttcattgg
cttgctgtca gggtactggg atggggggag gtgcatgggt tggggtggcc accaggtggt
gctgtgccac agcgggcagc ccctctggaa atgactggca tcataaaatc tgtcttcata
cccga

(SEQ ID NO:28), a different isoform of the RNA, or a nucleic acid having at least 80%, 82%, 85%, 86%, 88%, 90%, 92%, 94%, 95%, 97%, 98% or 99% nucleic acid sequence identity thereto, is detected.

In one embodiment, a product encoded by Homo sapiens ribosomal protein L6 (RPL6), mRNA NCBI Reference Sequence: NM_001029.5, e.g.,

MAGEKVEKPDTKEKKPEAKKVDAGGKVKKGNLKAKKPKKGKPHC
SRNPVLVRGIGRYSRSAMYSRKAMYKRKYSAAKSKVEKKKKEKVLATVTKPVGGDKNG
GTRVVKLRKMPRYYPTEDVPRKLLSHGKKPFSQHVRKLRASITPGTILIILTGRHRGK
RVVFLKQLASGLLLVTGPLVLNRVPLRRTHQKFVIATSTKIDISNVKIPKHLTDAYFK
KKKLRKPRHQEGEIFDTEKEKYEITEQRKIDQKAVDSQILPKIKAIPQLQGYLRSVFA
LTNGIYPHKLVF

(SEQ ID NO:29), a different isoform of the protein, or a polypeptide having at least 80%, 82%, 85%, 86%, 88%, 90%, 92%, 94%, 95%, 97%, 98% or 99% amino acid sequence identity thereto,
or a gene comprising or RNA corresponding to

ctctttccca tcttgcaaga tggcgggtga aaaagttgag aagccagata ctaaagagaa
gaaacccgaa gccaagaagg ttgatgctgg tggcaaggtg aaaaagggta acctcaaagc
taaaaagccc aagaagggga agccccattg cagccgcaac cctgtccttg tcagaggaat
tggcaggtat tcccgatctg ccatgtattc cagaaaggcc atgtacaaga ggaagtactc
agccgctaaa tccaaggttg aaaagaaaaa gaaggagaag gttctcgcaa ctgttacaaa
accagttggt ggtgacaaga acggcggtac ccgggtggtt aaacttcgca aaatgcctag
atattatcct actgaagatg tgcctcgaaa gctgttgagc cacggcaaaa aacccttcag
tcagcacgtg agaaaactgc gagccagcat tacccccggg accattctga tcatcctcac
tggacgccac aggggcaaga gggtggtttt cctgaagcag ctggctagtg gcttattact
tgtgactgga cctctggtcc tcaatcgagt tcctctacga agaacacacc agaaatttgt
cattgccact tcaaccaaaa tcgatatcag caatgtaaaa atcccaaaac atcttactga
tgcttacttc aagaagaaga agctgcggaa gcccagacac caggaaggtg agatcttcga
cacagaaaaa gagaaatatg agattacgga gcagcgcaag attgatcaga aagctgtgga
ctcacaaatt ttaccaaaaa tcaaagctat tcctcagctc cagggctacc tgcgatctgt
gtttgctctg acgaatggaa tttatcctca caaattggtg ttctaaatgt cttaagaacc
taattaaata gctgactaca t

(SEQ ID NO:30), a different isoform of the RNA, or a nucleic acid having at least 80%, 82%, 85%, 86%, 88%, 90%, 92%, 94%, 95%, 97%, 98% or 99% nucleic acid sequence identity thereto, is detected.

In one embodiment, a product encoded by Homo sapiens ribosomal protein L7a6 (RPL7A), mRNA NCBI Reference Sequence: NM_000972.3, e.g.,

MPKGKKAKGKKVAPAPAVVKKQEAKKVVNPLFEKRPKNFGIGQD
IQPKRDLTRFVKWPRYIRLQRQRAILYKRLKVPPAINQFTQALDRQTATQLLKLAHKY
RPETKQEKKQRLLARAEKKAAGKGDVPTKRPPVLRAGVNTVTTLVENKKAQLVVIAHD
VDPIELVVFLPALCRKMGVPYCIIKGKARLGRLVHRKTCTTVAFTQVNSEDKGALAKL
VEAIRTNYNDRYDEIRRHWGGNVLGPKSVARIAKLEKAKAKELATKLG

(SEQ ID NO:31), a different isoform of the protein, or a polypeptide having at least 80%, 82%, 85%, 86%, 88%, 90%, 92%, 94%, 95%, 97%, 98% or 99% amino acid sequence identity thereto,
or a gene comprising or RNA corresponding to

ctttctctct cctcccgccg cccaagatgc cgaaaggaaa gaaggccaag ggaaagaagg
tggctccggc cccagctgtc gtgaagaagc aggaggctaa gaaagtggtg aatcccctgt
ttgagaaaag gcctaagaat tttggcattg gacaggacat ccagcccaaa agagacctca
cccgctttgt gaaatggccc cgctatatca ggttgcagcg gcagagagcc atcctctata
agcggctgaa agtgcctcct gcgattaacc agttcaccca ggccctggac cgccaaacag
ctactcagct gcttaagctg gcccacaagt acagaccaga gacaaagcaa gagaagaagc
agagactgtt ggcccgggcc gagaagaagg ctgctggcaa aggggacgtc ccaacgaaga
gaccacctgt ccttcgagca ggagttaaca ccgtcaccac cttggtggag aacaagaaag
ctcagctggt ggtgattgca cacgacgtgg atcccatcga gctggttgtc ttcttgcctg
ccctgtgtcg taaaatgggg gtcccttact gcattatcaa gggaaaggca agactgggac
gtctagtcca caggaagacc tgcaccactg tcgccttcac acaggtgaac tcggaagaca
aaggcgcttt ggctaagctg gtggaagcta tcaggaccaa ttacaatgac agatacgatg
agatccgccg tcactggggt ggcaatgtcc tgggtcctaa gtctgtggct cgtatcgcca
agctcgaaaa ggcaaaggct aaagaacttg ccactaaact gggttaaatg tacactgttg
agttttctgt acataaaaat aattgaaata atacaaattt tccttca

(SEQ ID NO:32), a different isoform of the RNA, or a nucleic acid having at least 80%, 82%, 85%, 86%, 88%, 90%, 92%, 94%, 95%, 97%, 98% or 99% nucleic acid sequence identity thereto, is detected.

In one embodiment, a product encoded by Homo sapiens ribosomal protein S15a (RPS15A), mRNA NCBI Reference Sequence: NM_001030009.2, e.g.,

MVRMNVLADALKSINNAEKRGKRQVLIRPCSKVIVRFLTVMMKH
GYIGEFEIIDDHRAGKIVVNLTGRLNKCGVISPRFDVQLKDLEKWQNNL
LPSRQFGFIVLTTSAGIMDHEEARRKHTGGKILGFFF

(SEQ ID NO:33), a different isoform of the protein, or a polypeptide having at least 80%, 82%, 85%, 86%, 88%, 90%, 92%, 94%, 95%, 97%, 98% or 99% amino acid sequence identity thereto,
or a gene comprising or RNA corresponding to

ctctttccgc catctttccg cgccgccaca atggtgcgca tgaatgtcct ggcagatgct
ctcaagagta tcaacaatgc cgaaaagaga ggcaaacgcc aggtgcttat taggccgtgc
tccaaagtca tcgtccggtt tctcactgtg atgatgaagc atggttacat tggcgaattt
gaaatcattg atgaccacag agctgggaaa attgttgtga acctcacagg caggctaaac
aagtgtgggg tgatcagccc cagatttgac gtgcaactca aagacctgga aaaatggcag
aataatctgc ttccatcccg ccagtttggt ttcattgtac tgacaacctc agctggcatc
atggaccatg aagaagcaag acgaaaacac acaggaggga aaatcctggg attctttttc
tagggatgta atacatatat ttacaaataa aatgcctcat ggactctggt gcttccactt
ggtcgttttg agcctttaca gcagtgtagc cacagcttct gcggcagcat gcagttgctt
cgtttatcgg tgaatgcgat tccctgaagt gactaataca gccaagggaa aaagttctta
tgaaaccagt atgcctaaga aacagtcacc cctgctgtct gccaaaacca ggtatttgac
actaaatatt ttagttgtat ttcagttttt tttttttttt ttcttttttg gagacagagt
ctgactctat tgctcaggct ggagtgcagt ggcgcgatct tggcccactg caacctccac
ctcccgggtt caagtgattg tcctgtctca gccgcctgag taactgggat tacaagtgtg
tgtcgccaca tccggctaat ttttatattt tagtagagac agggtttcgc catgttgccc
aggctggtct tgagttctgg gcctcaagtg atcagcctac ctcggcctcc cgaagtgctg
ggattacagc cacgagccat tacacctggc ctatatttca gtattttcta ttagtttttg
atgaatttgt tttgcctggc taggattatt ctgtagatag gattttagat ctggcttttg
tcactgactg ctgtaataaa tacttgctag gaattttttt tttttttttt ttttttttaa
gacaaagtcg ctctgtcaca caggctggag tgcagtggca tgatcttggc tcactgcacc
tccgcttccc agattcaagt agttctcgtg cctcagcctc ctgagtagct gggattacag
gtgtgtgcca ccatgtctgg ctgatttttg tatttttagt agaggtgggg tttcaccatg
ttggccaggc tggtctcgaa ctcctgatct caagtgatct gcctgccttg gtctcccaaa
gtgctgggat tacaggtgtg accaccacgc cctgccttaa gaattgttcc aagagaatct
ggtgccactt gcaggtgccc attgaagtgc aatgggcact gttgatcact gaggaggtag
tgggtgctga cccggtgctg gggcctgtcc cctagtctct gctttgccct tggctagcta
ggtggtgtgc caagtgggga gagaagctac cttattaagg ggcatggatc agcttcctga
aaggagggcc tgcctctgta agatatggga agtcgctgag aatgttacag aacggcccta
gagatggggc agataacggc ccccatttgt gagaagtgag ttgggaggca tgtttggggc
ctctgatgtt tgggaggctg tgggtaatta acatgagttt tggggtccag cagcagaatt
caggtttcct cttccactca gtaacctcag catccgtatc tgtaatggga atgatacaaa
acctatcccc aagttgaggg aaaaatgaga ttgtgtaaag cgcacttggc acatgacagt
cacaagcatg gggacagtga gtccagaagg attttcttat gccagcattg taagccctag
gatcacaggg ctctggcttg tttaaccatc gtgtctctgg tccctagcct gcaaacctgg
tgtgtaggga tgcctcagtc gcttacatgt tgattgagtg aatcgtcggt ttctttctgg
acactgactt caaaaataaa ataggatatg aaaatgg

(SEQ ID NO:34), a different isoform of the RNA, or a nucleic acid having at least 80%, 82%, 85%, 86%, 88%, 90%, 92%, 94%, 95%, 97%, 98% or 99% nucleic acid sequence identity thereto, is detected.

In one embodiment, a product encoded by Homo sapiens ribosomal protein S28a (RPS28), mRNA NCBI Reference Sequence: NM_001031.5, e.g.,

MDTSRVQPIKLARVTKVLGRTGSQGQCTQVRVEFMDDTSRSIIR
NVKGPVREGDVLTLLESEREARRLR

(SEQ ID NO:35), a different isoform of the protein, or a polypeptide having at least 80%, 82%, 85%, 86%, 88%, 90%, 92%, 94%, 95%, 97%, 98% or 99% amino acid sequence identity thereto,
or a gene comprising or RNA corresponding to

actcctctcc gccagaccgc cgccgcgccg ccatcatgga caccagccgt gtgcagccta
tcaagctggc cagggtcacc aaggtcctgg gcaggaccgg ttctcaggga cagtgcacgc
aggtgcgcgt ggaattcatg gacgacacga gccgatccat catccgcaat gtaaaaggcc
ccgtgcgcga gggcgacgtg ctcacccttt tggagtcaga gcgagaagcc cggaggttgc
gctgagcttg gctgctcgct gggtcttgga tgtcgggttc gaccacttgg ccgatgggaa
tggtctgtca cagtctgctc cttttttttg tccgccacac gtaactgaga tgctccttta
aataaagcgt ttgtgtttca agttaactca ggttcttgtc tgggttatac gactagggtt
tctccaggtt tcttgagtgg ctcccaggcg gtcaccgatc ctccgcactc tggaaatcct
ggccgtgcgg tcttgccaaa cgaagctttt cctttttgag gcggggggtc gtgtttgtcg
attgcaccct ctaccccaaa caaaacacaa gcgtagtagg aatgttttat tagcaaagaa
gtttcagaga gtgggtggat cagggctcta tcacttggtc cccacctcac cttggtgggg
ccagagtgag ccccttcctg ccacagtcac cccaactgaa attgcctttc tcttcggcca
gtgttagcct ctgagcaggg gaccctggac ccttctgtgc gccaaaggct gaggtgactg
acgaggagat ctccccacag ctaggtgtag tgagccagac gaggcagctt actgaacctg
ggggttctct ccattgtcac cgcattctcc ttcaccaggt gtggctgtct gggagccagg
gggtgactcg ctctggagag aggggaaaag aggggggcct gctgcaatct ccttgaggca
ggaaacgtgg gattcagccc cagcctcact tagtggaggt tcttttacca tggacccagg
ctgcctggtt tgtatccaac ctctgcccct tctgacctgg aagaggcgct tgaccttect
cccacatccc ttccagtggg gtgagtacag gtgttcctca gtttacaatg ggttacattc
cggtgagtac atcataggtt gaaagtattg caagttgaaa tgtgtttaat acacctaatc
tcccaaacat cacagcttag catggtccat cttaagcttg ttcagaacgc cttagcctgt
agttggggaa actcgtctaa cacgaagcct gttttaataa agtattgaat gtcttatgta
atttattgaa

(SEQ ID NO:36), a different isoform of the RNA, or a nucleic acid having at least 80%, 82%, 85%, 86%, 88%, 90%, 92%, 94%, 95%, 97%, 98% or 99% nucleic acid sequence identity thereto, is detected.

In one embodiment, a product encoded by Homo sapiens ribosomal protein S5 (RPS5), mRNA NCBI Reference Sequence: NM_001009.4, e.g.,

MTEWETAAPAVAETPDIKLFGKWSTDDVQINDISLQDYIAVKEK
YAKYLPHSAGRYAAKRFRKAQCPIVERLTNSMMMHGRNNGKKLMTVRIV
KHAFEIIHLLTGENPLQVLVNAIINSGPREDSTRIGRAGTVRRQAVDVS
PLRRVNQAIWLLCTGAREAAFRNIKTIAECLADELINAAKGSSNSYAIK
KKDELERVAKSNR

(SEQ ID NO:37), a different isoform of the protein, or a polypeptide having at least 80%, 82%, 85%, 86%, 88%, 90%, 92%, 94%, 95%, 97%, 98% or 99% amino acid sequence identity thereto,
or a gene comprising or RNA corresponding to

ctcttcctgt ctgtaccagg gcggcgcgtg gtctacgccg agtgacagag acgctcaggc
tgtgttctca ggatgaccga gtgggagaca gcagcaccag cggtggcaga gaccccagac
atcaagctct ttgggaagtg gagcaccgat gatgtgcaga tcaatgacat ttccctgcag
gattacattg cagtgaagga gaagtatgcc aagtacctgc ctcacagtgc agggcggtat
gccgccaaac gcttccgcaa agctcagtgt cccattgtgg agcgcctcac taactccatg
atgatgcacg gccgcaacaa cggcaagaag ctcatgactg tgcgcatcgt caagcatgcc
ttcgagatca tacacctgct cacaggcgag aaccctctgc aggtcctggt gaacgccatc
atcaacagtg gtccccggga ggactccaca cgcattgggc gcgccgggac tgtgagacga
caggctgtgg atgtgtcccc cctgcgccgt gtgaaccagg ccatctggct gctgtgcaca
ggcgctcgtg aggctgcctt ccggaacatt aagaccattg ctgagtgcct ggcagatgag
ctcatcaatg ctgccaaggg ctcctcgaac tcctatgcca ttaagaagaa ggacgagctg
gagcgtgtgg ccaagtccaa ccgctgattt tcccagctgc tgcccaataa acctgtctgc
cctttggggc agtcccagcc a

(SEQ ID NO:38), a different isoform of the RNA, or a nucleic acid having at least 80%, 82%, 85%, 86%, 88%, 90%, 92%, 94%, 95%, 97%, 98% or 99% nucleic acid sequence identity thereto, is detected.

In one embodiment, a product encoded by Homo sapiens ribosomal protein S7 (RPS7), mRNA NCBI Reference Sequence: NM_001011.4, e.g.,

MFSSSAKIVKPNGEKPDEFESGISQALLELEMNSDLKAQLRELN
ITAAKEIEVGGGRKAIIIFVPVPQLKSFQKIQVRLVRELEKKFSGKHVV
FIAQRRILPKPTRKSRTKNKQKRPRSRTLTAVHDAILEDLVFPSEIVGK
RIRVKLDGSRLIKVHLDKAQQNNVEHKVETFSGVYKKLTGKDVNFEFPE
FQL

(SEQ ID NO:39), a different isoform of the protein, or a polypeptide having at least 80%, 82%, 85%, 86%, 88%, 90%, 92%, 94%, 95%, 97%, 98% or 99% amino acid sequence identity thereto,
or a gene comprising or RNA corresponding to

ctcgcgctgt ttccgcctct tgccttcgga cgccggattt tgacgtgctc tcgcgagatt
tgggtctctt cctaagccgg cgctcggcaa gttctcccag gagaaagcca tgttcagttc
gagcgccaag atcgtgaagc ccaatggcga gaagccggac gagttcgagt ccggcatctc
ccaggctctt ctggagctgg agatgaactc ggacctcaag gctcagctca gggagctgaa
tattacggca gctaaggaaa ttgaagttgg tggtggtcgg aaagctatca taatctttgt
tcccgttcct caactgaaat ctttccagaa aatccaagtc cggctagtac gcgaattgga
gaaaaagttc agtgggaagc atgtcgtctt tatcgctcag aggagaattc tgcctaagcc
aactcgaaaa agccgtacaa aaaataagca aaagcgtccc aggagccgta ctctgacagc
tgtgcacgat gccatccttg aggacttggt cttcccaagc gaaattgtgg gcaagagaat
ccgcgtcaaa ctagatggca gccggctcat aaaggttcat ttggacaaag cacagcagaa
caatgtggaa cacaaggttg aaactttttc tggtgtctat aagaagctca cgggcaagga
tgttaatttt gaattcccag agtttcaatt gtaaacaaaa atgactaaat aaaaagtata
tattcacaat ac

(SEQ ID NO:40), a different isoform of the RNA, or a nucleic acid having at least 80%, 82%, 85%, 86%, 88%, 90%, 92%, 94%, 95%, 97%, 98% or 99% nucleic acid sequence identity thereto, is detected.

In one embodiment, a product encoded by Homo sapiens ribosomal protein SA (RPSA), mRNA NCBI Reference Sequence: NM_001030009.2, e.g.,

MSGALDVLQMKEEDVLKFLAAGTHLGGTNLDFQMEQYIYKRKSD
GIYIINLKRTWEKLLLAARAIVAIENPADVSVISSRNTGQRAVLKFAAA
TGATPIAGRFTPGTFTNQIQAAFREPRLLVVTDPRADHQPLTEASYVNL
PTIALCNTDSPLRYVDIAIPCNNKGAHSVGLMWWMLAREVLRMRGTISR
EHPWEVMPDLYFYRDPEEIEKEEQAAAEKAVTKEEFQGEWTAPAPEFTA
TQPEVADWSEGVQVPSVPIQQFPTEDWSAQPATEDWSAAPTAQATEWVG
ATTDWS

(SEQ ID NO:41), a different isoform of the protein, or a polypeptide having at least 80%, 82%, 85%, 86%, 88%, 90%, 92%, 94%, 95%, 97%, 98% or 99% amino acid sequence identity thereto,
or a gene comprising or RNA corresponding to

cttttccgtg ctacctgcag aggggtccat acggcgttgt tctggattcc cgtcgtaact
taaagggaaa ttttcacaat gtccggagcc cttgatgtcc tgcaaatgaa ggaggaggat
gtccttaagt tccttgcagc aggaacccac ttaggtggca ccaatcttga cttccagatg
gaacagtaca tctataaaag gaaaagtgat ggcatctata tcataaatct caagaggacc
tgggagaagc ttctgctggc agctcgtgca attgttgcca ttgaaaaccc tgctgatgtc
agtgttatat cctccaggaa tactggccag agggctgtgc tgaagtttgc tgctgccact
ggagccactc caattgctgg ccgcttcact cctggaacct tcactaacca gatccaggca
gccttccggg agccacggct tettgtggtt actgacccca gggctgacca ccagcctctc
acggaggcat cttatgttaa cctacctacc attgcgctgt gtaacacaga ttctcctctg
cgctatgtgg acattgccat cccatgcaac aacaagggag ctcactcagt gggtttgatg
tggtggatgc tggctcggga agttctgcgc atgcgtggca ccatttcccg tgaacaccca
tgggaggtca tgcctgatct gtacttctac agagatcctg aagagattga aaaagaagag
caggctgctg ctgagaaggc agtgaccaag gaggaatttc agggtgaatg gactgctccc
gctcctgagt tcactgctac tcagcctgag gttgcagact ggtctgaagg tgtacaggtg
ccctctgtgc ctattcagca attccctact gaagactgga gcgctcagcc tgccacggaa
gactggtctg cagctcccac tgctcaggcc actgaatggg taggagcaac cactgactgg
tcttaagctg ttcttgcata ggctcttaag cagcatggaa aaatggttga tggaaaataa
acatcagttt ctaaaagttg tcttcattta gtttgctttt tactccagat cagaatacct
gggattgcat atcaaagcat aataataaat acatgtctcg acatgagttg tacttctaaa

(SEQ ID NO:42), a different isoform of the RNA, or a nucleic acid having at least 80%, 82%, 85%, 86%, 88%, 90%, 92%, 94%, 95%, 97%, 98% or 99% nucleic acid sequence identity thereto, is detected.

In one embodiment, a product encoded by Homo sapiens Baculoviral IAP repeat-containing protein 7 (BIRC7), mRNA NCBI Reference Sequence: NM_139317.3, e.g.,

MGPKDSAKCLHRGPQPSHWAAGDGPTQERCGPRSLGSPVLGLDT
CRAWDHVDGQILGQLRPLTEEEEEEGAGATLSRGPAFPGMGSEELRLAS
FYDWPLTAEVPPELLAAAGFFHTGHQDKVRCFFCYGGLQSWKRGDDPWT
EHAKWFPSCQFLLRSKGRDFVHSVQETHSQLLGSWDPWEEPEDAAPVAP
SVPASGYPELPTPRREVQSESAQEPGGVSPAEAQRAWWVLEPPGARDVE
AQLRRLQEERTCKVCLDRAVSIVFVPCGHLVCAECAPGLQLCPICRAPV
RSRVRTELS

(SEQ ID NO:43), a different isoform of the protein, or a polypeptide having at least 80%, 82%, 85%, 86%, 88%, 90%, 92%, 94%, 95%, 97%, 98% or 99% amino acid sequence identity thereto,
or a gene comprising or RNA corresponding to

acttccagaa agctgtgggc cctgggatac tcccctccca gggtgtctgg tggcaggcct
gtgcctatcc ctgctgtccc cagggtgggc cccgggggtc aggagctcca gaagggccag
ctgggcatat tctgagattg gccatcagcc cccatttctg ctgcaaacct ggtcagagcc
agtgttccct ccatgggacc taaagacagt gccaagtgcc tgcaccgtgg accacagccg
agccactggg cagccggtga tggtcccacg caggagcgct gtggaccccg ctctctgggc
agccctgtcc taggcctgga cacctgcaga gcctgggacc acgtggatgg gcagatectg
ggccagctgc ggcccctgac agaggaggaa gaggaggagg gcgccggggc caccttgtcc
agggggcctg ccttccccgg catgggctct gaggagttgc gtctggcctc cttctatgac
tggccgctga ctgctgaggt gccacccgag ctgctggctg ctgccggctt cttccacaca
ggccatcagg acaaggtgag gtgcttcttc tgctatgggg gcctgcagag ctggaagcgc
ggggacgacc cctggacgga gcatgccaag tggttcccca gctgtcagtt cctgctccgg
tcaaaaggaa gagactttgt ccacagtgtg caggagactc actcccagct gctgggctcc
tgggacccgt gggaagaacc ggaagacgca gcccctgtgg ccccctccgt ccctgcctct
gggtaccctg agctgcccac acccaggaga gaggtccagt ctgaaagtgc ccaggagcca
ggaggggtca gtccagccga ggcccagagg gcgtggtggg ttcttgagcc cccaggagcc
agggatgtgg aggcgcagct gcggcggctg caggaggaga ggacgtgcaa ggtgtgcctg
gaccgcgccg tgtccatcgt ctttgtgccg tgcggccacc tggtctgtgc tgagtgtgcc
cccggcctgc agctgtgccc catctgcaga gcccccgtcc gcagccgcgt gcgcaccttc
ctgtcctagg ccaggtgcca tggccggcca ggtgggctgc agagtgggct ccctgcccct
ctctgcctgt tctggactgt gttctgggcc tgctgaggat ggcagagctg gtgtccatcc
agcactgacc agccctgatt ccccgaccac cgcccagggt ggagaaggag gcccttgctt
ggcgtggggg atggcttaac tgtacctgtt tggatgcttc tgaatagaaa taaagtgggt
tttccctgga ggta

(SEQ ID NO:44), a different isoform of the RNA, or a nucleic acid having at least 80%, 82%, 85%, 86%, 88%, 90%, 92%, 94%, 95%, 97%, 98% or 99% nucleic acid sequence identity thereto, is detected.

In one embodiment, a product encoded by Homo sapiens Cadherin-3 (CDH3)), mRNA NCBI Reference Sequence: NM_001793.6, e.g.,

MGLPRGPLASLLLLQVCWLQCAASEPCRAVFREAEVTLEAGGAE
QEPGQALGKVFMGCPGQEPALFSTDNDDFTVRNGETVQERRSLKERNPL
KIFPSKRILRRHKRDWVVAPISVPENGKGPFPQRLNQLKSNKDRDTKIF
YSITGPGADSPPEGVFAVEKETGWLLLNKPLDREEIAKYELFGHAVSEN
GASVEDPMNISIIVTDQNDHKPKFTQDTFRGSVLEGVLPGTSVMQVTAT
DEDDAIYTYNGVVAYSIHSQEPKDPHDLMFTIHRSTGTISVISSGLDRE
KVPEYTLTIQATDMDGDGSTTTAVAVVEILDANDNAPMEDPQKYEAHVP
ENAVGHEVQRLTVTDLDAPNSPAWRATYLIMGGDDGDHFTITTHPESNQ
GILTTRKGLDFEAKNQHTLYVEVTNEAPFVLKLPTSTATIVVHVEDVNE
APVFVPPSKVVEVQEGIPTGEPVCVYTAEDPDKENQKISYRILRDPAGW
LAMDPDSGQVTAVGTLDREDEQFVRNNIYEVMVLAMDNGSPPTTGTGTL
LLTLIDVNDHGPVPEPRQITICNQSPVRQVLNITDKDLSPHTSPFQAQL
TDDSDIYWTAEVNEEGDTVVLSLKKFLKQDTYDVHLSLSDHGNKEQLTV
IRATVCDCHGHVETCPGPWKGGFILPVLGAVLALLFLLLVLLLLVRKKR
KIKEPLLLPEDDTRDNVFYYGEEGGGEEDQDYDITQLHRGLEARPEVVL
RNDVAPTIIPTPMYRPRPANPDEIGNFIIENLKAANTDPTAPPYDTLLV
EDYEGSGSDAASLSSLTSSASDQDQDYDYLNEWGSRFKKLADMYGGGED
D

(SEQ ID NO:45), a different isoform of the protein, or a polypeptide having at least 80%, 82%, 85%, 86%, 88%, 90%, 92%, 94%, 95%, 97%, 98% or 99% amino acid sequence identity thereto,
or a gene comprising or RNA corresponding to

aaaggggcaa gagctgagcg gaacaccggc ccgccgtcgc ggcagctgct tcacccctct
ctctgcagcc atggggctcc ctcgtggacc tctcgcgtct ctcctccttc tccaggtttg
ctggctgcag tgcgcggcct ccgagccgtg ccgggcggtc ttcagggagg ctgaagtgac
cttggaggcg ggaggcgcgg agcaggagcc cggccaggcg ctggggaaag tattcatggg
ctgccctggg caagagccag ctctgtttag cactgataat gatgacttca ctgtgcggaa
tggcgagaca gtccaggaaa gaaggtcact gaaggaaagg aatccattga agatcttccc
atccaaacgt atottacgaa gacacaagag agattgggtg gttgctccaa tatctgtccc
tgaaaatggc aagggtccct tcccccagag actgaatcag ctcaagtcta ataaagatag
agacaccaag attttctaca gcatcacggg gccgggggca gacagccccc ctgagggtgt
cttcgctgta gagaaggaga caggctggtt gttgttgaat aagccactgg accgggagga
gattgccaag tatgagctct ttggccacgc tgtgtcagag aatggtgcct cagtggagga
ccccatgaac atctccatca tcgtgaccga ccagaatgac cacaagccca agtttaccca
ggacaccttc cgagggagtg tcttagaggg agtcctacca ggtacttctg tgatgcaggt
gacagccacg gatgaggatg atgccatcta cacctacaat ggggtggttg cttactccat
ccatagccaa gaaccaaagg acccacacga cctcatgttc accattcacc ggagcacagg
caccatcagc gtcatctcca gtggcctgga ccgggaaaaa gtccctgagt acacactgac
catccaggcc acagacatgg atggggacgg ctccaccacc acggcagtgg cagtagtgga
gatccttgat gccaatgaca atgctcccat gtttgacccc cagaagtacg aggcccatgt
gcctgagaat gcagtgggcc atgaggtgca gaggctgacg gtcactgatc tggacgcccc
caactcacca gcgtggcgtg ccacctacct tatcatgggc ggtgacgacg gggaccattt
taccatcacc acccaccctg agagcaacca gggcatcctg acaaccagga agggtttgga
ttttgaggcc aaaaaccagc acaccctgta cgttgaagtg accaacgagg ccccttttgt
gctgaagctc ccaacctcca cagccaccat agtggtccac gtggaggatg tgaatgaggc
acctgtgttt gtcccaccct ccaaagtcgt tgaggtccag gagggcatcc ccactgggga
gcctgtgtgt gtctacactg cagaagaccc tgacaaggag aatcaaaaga tcagctaccg
catcctgaga gacccagcag ggtggctagc catggaccca gacagtgggc aggtcacagc
tgtgggcacc ctcgaccgtg aggatgagca gtttgtgagg aacaacatct atgaagtcat
ggtcttggcc atggacaatg gaagccctcc caccactggc acgggaaccc ttctgctaac
actgattgat gtcaatgacc atggcccagt ccctgagccc cgtcagatca ccatctgcaa
ccaaagccct gtgcgccagg tgctgaacat cacggacaag gacctgtctc cccacacctc
ccctttccag gcccagctca cagatgactc agacatctac tggacggcag aggtcaacga
ggaaggtgac acagtggtct tgtccctgaa gaagttcctg aagcaggata catatgacgt
gcacctttct ctgtctgacc atggcaacaa agagcagctg acggtgatca gggccactgt
gtgcgactgc catggccatg tcgaaacctg ccctggaccc tggaagggag gtttcatcct
ccctgtgctg ggggctgtcc tggctctgct gttcctcctg ctggtgctgc ttttgttggt
gagaaagaag cggaagatca aggagcccct cctactccca gaagatgaca cccgtgacaa
cgtcttctac tatggcgaag aggggggtgg cgaagaggac caggactatg acatcaccca
gctccaccga ggtctggagg ccaggccgga ggtggttctc cgcaatgacg tggcaccaac
catcatcccg acacccatgt accgtcctcg gccagccaac ccagatgaaa tcggcaactt
tataattgag aacctgaagg cggctaacac agaccccaca gccccgccct acgacaccct
cttggtgttc gactatgagg gcagcggctc cgacgccgcg tccctgagct ccctcacctc
ctccgcctcc gaccaagacc aagattacga ttatctgaac gagtggggca gccgcttcaa
gaagctggca gacatgtacg gtggcgggga ggacgactag gcggcctgcc tgcagggctg
gggaccaaac gtcaggccac agagcatctc caaggggtct cagttccccc ttcagctgag
gacttcggag cttgtcagga agtggccgta gcaacttggc ggagacaggc tatgagtctg
acgttagagt ggtggcttcc ttagcctttc aggatggagg aatgtgggca gtttgacttc
agcactgaaa acctctccac ctgggccagg gttgcctcag aggccaagtt tccagaagcc
tcttacctgc cgtaaaatgc tcaaccctgt gtcctgggcc tgggcctgct gtgactgacc
tacagtggac tttctctctg gaatggaacc ttcttaggcc tcctggtgca acttaatttt
tttttttaat gctatcttca aaacgttaga gaaagttctt caaaagtgca gcccagagct
gctgggccca ctggccgtcc tgcatttctg gtttccagac cccaatgcct cccattcgga
tggatctctg cgtttttata ctgagtgtgc ctaggttgcc ccttattttt tattttccct
gttgcgttgc tatagatgaa gggtgaggac aatcgtgtat atgtactaga acttttttat
taaagaaact tttcccagag gtgcctgggg agtgaactgt tttctaaata gaaggtttat
tggcatctaa ctcacatacc ataccattca cttgtttaac gtttacaatt caatggtttt
tagaattttc agagttctgc aaaaagagtg ggctctgttt accctgctgg cttcacccaa
gcttcctctc aatggcaggg gatactcagg gtcagcttcc atgcctaagt gggctcaggg
aggggagact tgccctcctt tgcaaagtca gttgctccat gagagagaac cattaacctc
ttaccccaag gctgagcccc tccacagccc cagcaaggte tettctggaa cagcggctgc
cctccctgca ggacagtgtg tgacgacttc ctcttctatg ctggaggggt ccctgttgaa
aggcaggggt tggggaacag ccagctctgc tacttgctag cacatttttt tttttttttt
tgagacggag tctcgttctg tcgcccaggc tggagtgcag tggtgtgatc ttggctcact
gcaagctctg cctcctgggt tcacgccatt ctcctgcctc agcctcccga gtagctggga
ctacaggtgc ctgccactat gcccagctaa ttttttttga gtgtgtgtgt ttttttagta
gagacggggt ttcactgtgt tagccaggat gtctcgatct cctgacctcg tgatccgccc
acgtcggcct cccaaagtgc taggattaca ggcttgagcc accgcgccca gcgctggcac
atcatttaac ctccagttgc ctcagatttt acatttacaa aatggggagt ttttgtggag
attaagtgaa ttaatatctg gcacatggtc catgcaatct gttagttggt aacagctacc
atttattcag tacttttaaa ggccagacag gacttcgatt atttcctcta aatcctcaca
atcaccctct gaggggactt tctcctttaa agaatggcca cattgtattt gtttttttaa
atgacatctg gtcatcatcg aaatcaagca aaacaaaatt aagagaacct acccaagatg
tcagtgaaat tggaacattc ctgacaatac cagggcataa atgcaggaat caggaatagg
cagcagtgat agaacaattc tgtttgtgcc cttgttaacg tgaagttcaa agtcatcttt
gcaattagcc aaaagaatct gaagtgaagc tgaggaaatt gctgatgttg aaataaacat
ttccttccat ga

(SEQ ID NO:46), a different isoform of the RNA, or a nucleic acid having at least 80%, 82%, 85%, 86%, 88%, 90%, 92%, 94%, 95%, 97%, 98% or 99% nucleic acid sequence identity thereto, is detected.

In one embodiment, a product encoded by Homo sapiens Dual specificity protein kinase CLK1 (CLK1), mRNA NCBI Reference Sequence: NM_004071.4, e.g.,

MRHSKRTYCPDWDDKDWDYGKWRSSSSHKRRKRSHSSAQENKRC
KYNHSKMCDSHYLESRSINEKDYHSRRYIDEYRNDYTQGCEPGHRQRDH
ESRYQNHSSKSSGRSGRSSYKSKHRIHHSTSHRRSHGKSHRRKRTRSVE
DDEEGHLICQSGDVLSARYEIVDTLGEGAFGKVVECIDHKAGGRHVAVK
IVKNVDRYCEAARSEIQVLEHLNTTDPNSTFRCVQMLEWFEHHGHICIV
FELLGLSTYDFIKENGFLPFRLDHIRKMAYQICKSVNFLHSNKLTHTDL
KPENILFVQSDYTEAYNPKIKRDERTLINPDIKVVDFGSATYDDEHHST
LVSTRHYRAPEVILALGWSQPCDVWSIGCILIEYYLGFTVFPTHDSKEH
LAMMERILGPLPKHMIQKTRKRKYFHHDRLDWDEHSSAGRYVSRRCKPL
KEFMLSQDVEHERLFDLIQKMLEYDPAKRITLREALKHPFFDLLKKSI

(SEQ ID NO:47), a different isoform of the protein, or a polypeptide having at least 80%, 82%, 85%, 86%, 88%, 90%, 92%, 94%, 95%, 97%, 98% or 99% amino acid sequence identity thereto,
or a gene comprising or RNA corresponding to

attttgttgt tggtgcgcga cgcagtcagc tgcgtgattc ccgtgattgc gttacaagct
ttgtctcctt cgacttggag tctttgtcca ggacgatgag acactcaaag agaacttact
gtcctgattg ggatgacaag gattgggatt atggaaaatg gaggagcagc agcagtcata
aaagaaggaa gagatcacat agcagtgccc aggagaacaa gcgctgcaaa tacaatcact
ctaaaatgtg tgatagccat tatttggaaa gcaggtctat aaatgagaaa gattatcata
gtcgacgcta cattgatgag tacagaaatg actacactca aggatgtgaa cctggacatc
gccaaagaga ccatgaaagc cggtatcaga accatagtag caagtcttct ggtagaagtg
gaagaagtag ttataaaagc aaacacagga ttcaccacag tacttcacat cgtcgttcac
atgggaagag tcaccgaagg aaaagaacca ggagtgtaga ggatgatgag gagggtcacc
tgatctgtca gagtggagac gtactaagtg caagatatga aattgttgat actttaggtg
aaggagcttt tggaaaagtt gtggagtgca tcgatcataa agcgggaggt agacatgtag
cagtaaaaat agttaaaaat gtggatagat actgtgaagc tgctcgctca gaaatacaag
acgacttcat taaagaaaat acagacccca acagtacttt ccgctgtgtc cagatgttgg
ttctggaaca tctgaataca cacatttgca ttgtttttga actattggga cttagtactt
aatggtttga gcatcatggt ggttttctac catttcgact ggatcatatc agaaagatgg
catatcagat atgcaagtct gtgaattttt tgcacagtaa taagttgact cacacagact
taaagcctga aaacatctta tttgtgcagt ctgactacac agaggcgtat aatcccaaaa
taaaacgtga tgaacgcacc ttaataaatc cagatattaa agttgtagac tttggtagtg
caacatatga tgacgaacat cacagtacat tggtatctac aagacattat agagcacctg
aagttatttt agccctaggg tggtcccaac catgtgatgt ctggagcata ggatgcattc
ttattgaata ctatcttggg tttaccgtat ttccaacaca cgatagtaag gagcatttag
caatgatgga aaggattctt ggacctctac caaaacatat gatacagaaa accaggaaac
gtaaatattt tcaccacgat cgattagact gggatgaaca cagttctgcc ggcagatatg
tttcaagacg ctgtaaacct ctgaaggaat ttatgctttc tcaagatgtt gaacatgagc
gtctctttga cctcattcag aaaatgttgg agtatgatcc agccaaaaga attactctca
gagaagcctt aaagcatcct ttctttgacc ttctgaagaa aagtatatag atctgtaatt
ggacagctct ctcgaagaga tcttacagac tgtatcagtc taatttttaa attttaagtt
attttgtaca gctttgtaaa ttcttaacat ttttatattg ccatgtttat tttgtttggg
taatttggtt cattaagtac atagctaagg taatgaacat ctttttcagt aattgtaaag
tgatttattc agaataaatt ttttgtgctt atgaagttga tatgtatctg aacagtttgt
tctaagtacc atttttcttc ctacttctat taaagaatgg acataga

(SEQ ID NO:48), a different isoform of the RNA, or a nucleic acid having at least 80%, 82%, 85%, 86%, 88%, 90%, 92%, 94%, 95%, 97%, 98% or 99% nucleic acid sequence identity thereto, is detected.

In one embodiment, a product encoded by Homo sapiens Chondroitin sulfate proteoglycan 4 (CSPG4), mRNA NCBI Reference Sequence; NM_001897.5, e.g.,

MQSGPRPPLPAPGLALALTLTMLARLASAASFFGENHLEVPVAT
ALTDIDLQLQFSTSQPEALLLLAAGPADHLLLQLYSGRLQVRLVLGQEELRLQTPAET
LLSDSIPHTVVLTVVEGWATLSVDGFLNASSAVPGAPLEVPYGLFVGGTGTLGLPYLR
GTSRPLRGCLHAATLNGRSLLRPLTPDVHEGCAEEFSASDDVALGFSGPHSLAAFPAW
GTQDEGTLEFTLTTQSRQAPLAFQAGGRRGDFIYVDIFEGHLRAVVEKGQGTVLLHNS
VPVADGQPHEVSVHINAHRLEISVDQYPTHTSNRGVLSYLEPRGSLLLGGLDAEASRH
LQEHRLGLTPEATNASLLGCMEDLSVNGQRRGLREALLTRNMAAGCRLEEEEYEDDAY
GHYEAFSTLAPEAWPAMELPEPCVPEPGLPPVFANFTQLLTISPLVVAEGGTAWLEWR
HVQPTLDLMEAELRKSQVLFSVTRGARHGELELDIPGAQARKMFTLLDVVNRKARFIH
DGSEDTSDQLVLEVSVTARVPMPSCLRRGQTYLLPIQVNPVNDPPHIIFPHGSLMVIL
EHTQKPLGPEVFQAYDPDSACEGLTFQVLGTSSGLPVERRDQPGEPATEFSCRELEAG
SLVYVHRGGPAQDLTFRVSDGLQASPPATLKVVAIRPAIQIHRSTGLRLAQGSAMPIL
PANLSVETNAVGQDVSVLFRVTGALQFGELQKQGAGGVEGAEWWATQAFHQRDVEQGR
VRYLSTDPQHHAYDTVENLALEVQVGQEILSNLSFPVTIQRATVWMLRLEPLHTQNTQ
QETLTTAHLEATLEEAGPSPPTFHYEVVQAPRKGNLQLQGTRLSDGQGFTQDDIQAGR
VTYGATARASEAVEDTFRFRVTAPPYFSPLYTFPIHIGGDPDAPVLTNVLLVVPEGGE
GVLSADHLFVKSLNSASYLYEVMERPRHGRLAWRGTQDKTTMVTSFTNEDLLRGRLVY
QHDDSETTEDDIPFVATRQGESSGDMAWEEVRGVERVAIQPVNDHAPVQTISRIFHVA
RGGRRLLTTDDVAFSDADSGFADAQLVLTRKDLLFGSIVAVDEPTRPIYRFTQEDLRK
RRVLFVHSGADRGWIQLQVSDGQHQATALLEVQASEPYLRVANGSSLVVPQGGQGTID
TAVLHLDTNLDIRSGDEVHYHVTAGPRWGQLVRAGQPATAFSQQDLLDGAVLYSHNGS
LSPRDTMAFSVEAGPVHTDATLQVTIALEGPLAPLKLVRHKKIYVFQGEAAEIRRDQL
EAAQEAVPPADIVFSVKSPPSAGYLVMVSRGALADEPPSLDPVQSFSQEAVDTGRVLY
LHSRPEAWSDAFSLDVASGLGAPLEGVLVELEVLPAAIPLEAQNFSVPEGGSLTLAPP
LLRVSGPYFPTLLGLSLQVLEPPQHGALQKEDGPQARTLSAFSWRMVEEQLIRYVHDG
SETLTDSFVLMANASEMDRQSHPVAFTVTVLPVNDQPPILTTNTGLQMWEGATAPIPA
EALRSTDGDSGSEDLVYTIEQPSNGRVVLRGAPGTEVRSFTQAQLDGGLVLESHRGTL
DGGFRFRLSDGEHTSPGHFFRVTAQKQVLLSLKGSQTLTVCPGSVQPLSSQTLRASSS
AGTDPQLLLYRVVRGPQLGRLFHAQQDSTGEALVNFTQAEVYAGNILYEHEMPPEPFW
EAHDTLELQLSSPPARDVAATLAVAVSFEAACPQRPSHLWKNKGLWVPEGQRARITVA
ALDASNLLASVPSPQRSEHDVLFQVTQFPSRGQLLVSEEPLHAGQPHFLQSQLAAGQL
VYAHGGGGTQQDGFHFRAHLQGPAGASVAGPQTSEAFAITVRDVNERPPQPQASVPLR
LTRGSRAPISRAQLSVVDPDSAPGEIEYEVQRAPHNGFLSLVGGGLGPVTRFTQADVD
SGRLAFVANGSSVAGIFQLSMSDGASPPLPMSLAVDILPSAIEVQLRAPLEVPQALGR
SSLSQQQLRVVSDREEPEAAYRLIQGPQYGHLLVGGRPTSAFSQFQIDQGEVVFAFTN
FSSSHDHFRVLALARGVNASAVVNVTVRALLHVWAGGPWPQGATLRLDPTVLDAGELA
NRTGSVPRFRLLEGPRHGRVVRVPRARTEPGGSQLVEQFTQQDLEDGRLGLEVGRPEG
RAPGPAGDSLTLELWAQGVPPAVASLDFATEPYNAARPYSVALLSVPEAARTEAGKPE
SSTPTGEPGPMASSPEPAVAKGGFLSFLEANMFSVIIPMCLVLLLLALILPLLFYLRK
RNKTGKHDVQVLTAKPRNGLAGDTETFRKVEPGQAIPLTAVPGQGPPPGGQPDPELLQ
FCRTPNPALKNGQYWV

(SEQ ID NO:49), a different isoform of the protein, or a polypeptide having at least 80%, 82%, 85%, 86%, 88%, 90%, 92%, 94%, 95%, 97%, 98% or 99% amino acid sequence identity thereto,
or a gene comprising or RNA corresponding to

gcgcccagga gcagagccgc gctcgctcca ctcagctccc agctcccagg actccgctgg
ctcctcgcaa gtcctgccgc ccagcccgcc gggatgcagt ccgggccgcg gcccccactt
ccagcccccg gcctggcctt ggctttgacc ctgactatgt tggccagact tgcatccgcg
gcttccttct tcggtgagaa ccacctggag gtgcctgtgg ccacggctct gaccgacata
gacctgcagc tgcagttctc cacgtcccag cccgaagccc tccttctcct ggcagcaggc
ccagctgacc acctcctgct gcagctctac tctggacgcc tgcaggtcag acttgttctg
ggccaggagg agctgaggct gcagactcca gcagagacgc tgctgagtga ctccatcccc
cacactgtgg tgctgactgt cgtagagggc tgggccacgt tgtcagtcga tgggtttctg
aacgcctcct cagcagtccc aggagccccc ctagaggtcc cctatgggct ctttgttggg
ggcactggga cccttggcct gccctacctg aggggaacca gccgacccct gaggggttgc
ctccatgcag ccaccctcaa tggccgcagc ctcctccggc ctctgacccc cgatgtgcat
gagggctgtg ctgaagagtt ttctgccagt gatgatgtgg ccctgggctt ctctgggccc
cactctctgg ctgccttccc tgcctggggc actcaggacg aaggaaccct agagtttaca
ctcaccacac agagccggca ggcacccttg gccttccagg cagggggccg gcgtggggac
ttcatctatg tggacatatt tgagggccac ctgcgggccg tggtggagaa gggccagggt
accgtattgc tccacaacag tgtgcctgtg gccgatgggc agccccatga ggtcagtgtc
cacatcaatg ctcaccggct ggaaatctcc gtggaccagt accctacgca tacttcgaac
cgaggagtcc tcagctacct ggagccacgg ggcagtctcc ttctcggggg gctggatgca
gaggcctctc gtcacctcca ggaacaccgc ctgggcctga caccagaggc caccaatgcc
tccctgctgg gctgcatgga agacctcagt gtcaatggcc agaggcgggg gctgcgggaa
gctttgctga cgcgcaacat ggcagccggc tgcaggctgg aggaggagga gtatgaggac
gatgcctatg gacattatga agctttctcc accctggccc ctgaggcttg gccagccatg
gagctgcctg agccatgcgt gcctgagcca gggctgcctc ctgtctttgc caatttcacc
cagctgctga ctatcagccc actggtggtg gccgaggggg gcacagcctg gcttgagtgg
aggcatgtgc agcccacgct ggacctgatg gaggctgagc tgcgcaaatc ccaggtgctg
ttcagcgtga cccgaggggc acgccatggc gagctcgagc tggacatccc gggagcccag
gcacgaaaaa tgttcaccct cctggacgtg gtgaaccgca aggcccgctt catccacgat
ggctctgagg acacctccga ccagctggtg ctggaggtgt cggtgacggc tcgggtgccc
atgccctcat gccttcggag gggccaaaca tacctcctgc ccatccaggt caaccctgtc
aatgacccac cccacatcat cttcccacat ggcagcctca tggtgatcct ggaacacacg
cagaagccgc tggggcctga ggttttccag gcctatgacc cggactctgc ctgtgagggc
ctcaccttcc aggtccttgg cacctcctct ggcctccccg tggagcgccg agaccagcct
ggggagccgg cgaccgagtt ctcctgccgg gagttggagg ccggcagcct agtctatgtc
caccgcggtg gtcctgcaca ggacttgacg ttccgggtca gcgatggact gcaggccagc
cccccggcca cgctgaaggt ggtggccatc cggccggcca tacagatcca ccgcagcaca
gggttgcgac tggcccaagg ctctgccatg cccatcttgc ccgccaacct gtcggtggag
accaatgccg tggggcagga tgtgagcgtg ctgttccgcg tcactggggc cctgcagttt
ggggagctgc agaagcaggg ggcaggtggg gtggagggtg ctgagtggtg ggccacacag
gcgttccacc agcgggatgt ggagcagggc cgcgtgaggt acctgagcac tgacccacag
caccacgctt acgacaccgt ggagaacctg gccctggagg tgcaggtggg ccaggagatc
ctgagcaatc tgtccttccc agtgaccatc cagagagcca ctgtgtggat gctgcggctg
gagccactgc acactcagaa cacccagcag gagaccctca ccacagccca cctggaggcc
accctggagg aggcaggccc aagcccccca accttccatt atgaggtggt tcaggctccc
aggaaaggca accttcaact acagggcaca aggctgtcag atggccaggg cttcacccag
gatgacatac aggctggccg ggtgacctat ggggccacag cacgtgcctc agaggcagtc
gaggacacct tccgtttccg tgtcacagct ccaccatatt tctccccact ctataccttc
cccatccaca ttggtggtga cccagatgcg cctgtcctca ccaatgtcct cctcgtggtg
cctgagggtg gtgagggtgt cctctctgct gaccacctct ttgtcaagag tctcaacagt
gccagctacc tctatgaggt catggagcgg ccccgccatg ggaggttggc ttggcgtggg
acacaggaca agaccactat ggtgacatcc ttcaccaatg aagacctgtt gcgtggccgg
ctggtctacc agcatgatga ctccgagacc acagaagatg atatcccatt tgttgctacc
cgccagggcg agagcagtgg tgacatggcc tgggaggagg tacggggtgt cttccgagtg
gccatccagc ccgtgaatga ccacgcccct gtgcagacca tcagccggat cttccatgtg
gcccggggtg ggcggcggct gctgactaca gacgacgtgg ccttcagcga tgctgactcg
ggctttgctg acgcccagct ggtgcttacc cgcaaggacc tcctctttgg cagtatcgtg
gccgtagatg agcccacgcg gcccatctac cgcttcaccc aggaggacct caggaagagg
cgagtactgt tcgtgcactc aggggctgac cgtggctgga tccagctgca ggtgtccgac
gggcaacacc aggccactgc gctgctggag gtgcaggcct cggaacccta cctccgtgtg
gccaacggct ccagccttgt ggtccctcaa ggaggccagg gcaccatcga cacggccgtg
ctccacctgg acaccaacct cgacatccgc agtggggatg aggtccacta ccacgtcaca
gctggccctc gctggggaca gctagtccgg gctggtcagc cagccacagc cttctcccag
caggacctgc tggatggggc cgttctctat agccacaatg gcagcctcag cccccgcgac
accatggcct tctccgtgga agcagggcca gtgcacacgg atgccaccct acaagtgacc
attgccctag agggcccact ggccccactg aagctggtcc ggcacaagaa gatctacgtc
ttccagggag aggcagctga gatcagaagg gaccagctgg aggcagccca ggaggcagtg
ccacctgcag acatcgtatt ctcagtgaag agcccaccga gtgccggcta cctggtgatg
gtgtcgcgtg gcgccttggc agatgagcca cccagcctgg accctgtgca gagcttctcc
caggaggcag tggacacagg cagggtcctg tacctgcact cccgccctga ggcctggagc
gatgccttct cgctggatgt ggcctcaggc ctgggtgctc ccctcgaggg cgtccttgtg
gagctggagg tgctgcccgc tgccatccca ctagaggcgc aaaacttcag cgtccctgag
ggtggcagcc tcaccctggc ccctccactg ctccgtgtct ccgggcccta cttccccact
ctcctgggcc tcagcctgca ggtgctggag ccaccccagc atggagccct gcagaaggag
gacggacctc aagccaggac cctcagcgcc ttctcctgga gaatggtgga agagcagctg
atccgctacg tgcatgacgg gagcgagaca ctgacagaca gttttgtcct gatggctaat
gcctccgaga tggatcgcca gagccatcct gtggccttca ctgtcactgt cctgcctgtc
aatgaccaac cccccatcct cactacaaac acaggcctgc agatgtggga gggggccact
gcgcccatcc ctgcggaggc tctgaggagc acggacggcg actctgggtc tgaggatctg
gtctacacca tcgagcagcc cagcaacggg cgggtagtgc tgcggggggc gccgggcact
gaggtgcgca gcttcacgca ggcccagctg gacggcgggc tcgtgctgtt ctcacacaga
ggaaccctgg atggaggctt ccgcttccgc ctctctgacg gcgagcacac ttcccccgga
cacttcttcc gagtgacggc ccagaagcaa gtgctcctct cgctgaaggg cagccagaca
ctgactgtct gcccagggtc cgtccagcca ctcagcagtc agaccctcag ggccagctcc
agcgcaggca ctgaccccca gctcctgctc taccgtgtgg tgcggggccc ccagctaggc
cggctgttcc acgcccagca ggacagcaca ggggaggccc tggtgaactt cactcaggca
gaggtctacg ctgggaatat tctgtatgag catgagatgc cccccgagcc cttttgggag
gcccatgata ccctagagct ccagctgtcc tcgccgcctg cccgggacgt ggccgccacc
cttgctgtgg ctgtgtcttt tgaggctgcc tgtccccagc gccccagcca cctctggaag
aacaaaggtc tctgggtccc cgagggccag cgggccagga tcaccgtggc tgctctggat
gcctccaatc tcttggccag cgttccatca ccccagcgct cagagcatga tgtgctcttc
caggtcacac agttccccag ccggggccag ctgttggtgt ccgaggagcc cctccatgct
gggcagcccc acttcctgca gtcccagctg gctgcagggc agctagtgta tgcccacggc
ggtgggggca cccagcagga tggcttccac tttcgtgccc acctccaggg gccagcaggg
gcctccgtgg ctggacccca aacctcagag gcctttgcca tcacggtgag ggatgtaaat
gagcggcccc ctcagccaca ggcctctgtc ccactccggc tcacccgagg ctctcgtgcc
cccatctccc gggcccagct gagtgtggtg gacccagact cagctcctgg ggagattgag
tacgaggtcc agcgggcacc ccacaacggc ttcctcagcc tggtgggtgg tggcctgggg
cccgtgaccc gcttcacgca agccgatgtg gattcagggc ggctggcctt cgtggccaac
gggagcagcg tggcaggcat cttccagctg agcatgtctg atggggccag cccacccctg
cccatgtccc tggctgtgga catcctacca tccgccatcg aggtgcagct gcgggcaccc
ctggaggtgc cccaagcttt ggggcgctcc tcactgagcc agcagcagct ccgggtggtt
tcagatcggg aggagccaga ggcagcatac cgcctcatcc agggacccca gtatgggcat
ctcctggtgg gcgggcggcc cacctcggcc ttcagccaat tccagataga ccagggcgag
gtggtctttg ccttcaccaa cttctcctcc tctcatgacc acttcagagt cctggcactg
gctaggggtg tcaatgcatc agccgtagtg aacgtcactg tgagggctct gctgcatgtg
tgggcaggtg ggccatggcc ccagggtgcc accctgcgcc tggaccccac cgtcctagat
gctggcgagc tggccaaccg cacaggcagt gtgccgcgct tccgcctcct ggagggaccc
cggcatggcc gcgtggtccg cgtgccccga gccaggacgg agcccggggg cagccagctg
gtggagcagt tcactcagca ggaccttgag gacgggaggc tggggctgga ggtgggcagg
ccagagggga gggcccccgg ccccgcaggt gacagtctca ctctggagct gtgggcacag
ggcgtcccgc ctgctgtggc ctccctggac tttgccactg agccttacaa tgctgcccgg
ccctacagcg tggccctgct cagtgtcccc gaggccgccc ggacggaagc agggaagcca
gagagcagca cccccacagg cgagccaggc cccatggcat ccagccctga gcccgctgtg
gccaagggag gcttcctgag cttccttgag gccaacatgt tcagcgtcat catccccatg
tgcctggtac ttctgctcct ggcgctcatc ctgcccctgc tcttctacct ccgaaaacgc
aacaagacgg gcaagcatga cgtccaggtc ctgactgcca agccccgcaa cggcctggct
ggtgacaccg agacctttcg caaggtggag ccaggccagg ccatcccgct cacagctgtg
cctggccagg ggccccctcc aggaggccag cctgacccag agctgctgca gttctgccgg
acacccaacc ctgcccttaa gaatggccag tactgggtgt gaggcctggc ctgggcccag
atgctgatcg ggccagggac aggcttgccc atgtcccggg ccccattgct tccatgcctg
gtgctgtctg agtatcccca gagcaagaga gacctggaga caccagggtg ggagggtcct
gggagatagt cccaggggtc cgggacagag tggagtcaag agctggaacc tccctcagct
cactccgagc ctggagaact gcaggggcca aggtggaggc aggcttaagt tcagtcctcc
tgccctggag ctggtttggg ctgtcaaaac cagggtaacc tcctacatgg gtcatgactc
tgggtcctgg gtctgtgacc ttgggtaagt cgcgcctgac ccaggctgct aagagggcaa
ggagaaggaa gtaccctggg gagggaaggg acagaggaag ctattcctgg cttttccact
ccaacccagg ccaccctttg tctctgcccc agagttgaga aaaaaacttc ctcccctggt
tttttaggga gatggtatcc cctggagtag agggcaagag gagagagcgc ctccagtcta
gaaggcataa gccaatagga taatatattc agggtgcagg gtgggtaggt tgctctgggg
atgggtttat ttaagggaga ttgcaaggaa gctatttaac atggtgctga gctagccagg
actgatggag cccctggggg tgtgggatgg aggagggtct gcagccagtt cattcccagg
gccccatctt gatgggccaa gggctaaaca tgcatgtgtc agtggctttg gagcaggtta
ggctggggct catcgagggt ctcaggccga ggccactgcg gtgccagtgc ccccctgagg
actagggcag gcagctgggg gcacttggtt ccatggagcc tggataaaca gtgctttgga
ggctctggac agctgtgtgg tgtttgtgtc ttaactatgc actgggccct tgtctgcgtc
ggcttgcata cagagggccc ctggggtcgg ccctccggcc tggcctcagc cagtgggatg
gacagggcca ggcaggcctc tgaacttcca cctcctgggg cctcccagac ctcctgtgcc
ccaccctaga tgggcaggtg ggccagtctt cgggtgatgg gaccaaaccc cttcagttca
gtagagaaag gctaggtcct ctacaaagag ctgcaagaca aaaattaaaa taaatgctcc
cccacctgtg

(SEQ ID NO:50), a different isoform of the RNA, or a nucleic acid having at least 80%, 82%, 85%, 86%, 88%, 90%, 92%, 94%, 95%, 97%, 98% or 99% nucleic acid sequence identity thereto, is detected.

In one embodiment, a product encoded by Homo sapiens Eukaryotic translation initiation factor 4B (EIF4B), mRNA NCBI Reference Sequence: NM_001300821.3, e.g.

MAASAKKKNKKGKTISLTDFLAEDGGTGGGSTYVSKPVSWADET
DDLEGDVSTTWHSNDDDVYRAPPIDRSILPTAPRAAREPNIDRSRLPKSPPYTAFLGN
LPYDVTEESIKEFFRGLNISAVRLPREPSNPERLKGFGYAEFEDLDSLLSALSLNEES
LGNRRIRVDVADQAQDKDRDDRSFGRDRNRDSDKTDTDWRARPATDSEDDYPPRRGDD
SFGDKYRDRYDSDRYRDGYRDGYRDGPRRDMDRYGGRDRYDDRGSRDYDRGYDSRIGS
GRRAFGSGYRRDDDYRGGGDRYEDRYDRRDDRSWSSRDDYSRDDYRRDDRGPPQRPKL
NLKPRSTPKEDDSSASTSQSTRAASIFGGAKPVDTAAREREVEERLQKEQEKLQRQLD
EPKLERRPRERHPSWRSEETQERERSRTGSESSQTGTSTTSSRSKSDQDARRRESEKS
LENETLNKEEDCHSPTSKPPKPDQPLKVMPAPPPKENAWVKRSSNPPARSQSSDTEQQ
SPTSGGGKVAPAQPSEEGPGRKDENKVDGMNAPKGQTGNSSRGPGDGGNRDHWKESDR
KDGKKDQDSRSAPEPKKPEENPASKESSASKYAALSVDGEDENEGEDYAE

(SEQ ID NO:51), a different isoform of the protein, or a polypeptide having at least 80%, 82%, 85%, 86%, 88%, 90%, 92%, 94%, 95%, 97%, 98% or 99% amino acid sequence identity thereto,
or a gene comprising or RNA corresponding to

cttttgcgtt ctctttccct ctcccaacat ggcggcctca gcaaaaaaga agaataagaa
ggggaagact atctccctaa cagactttct ggctgaggat gggggtactg gtggaggaag
cacctatgtt tccaaaccag tcagctgggc tgatgaaacg gatgacctgg aaggagatgt
ttcgaccact tggcacagta acgatgacga tgtgtatagg gcgcctccaa ttgaccgttc
catccttccc actgctccac gggctgctcg ggaacccaat atcgaccgga gccgtcttcc
caaatcgcca ccctacactg cttttctagg aaacctaccc tatgatgtta cagaagagtc
aattaaggaa ttctttcgag gattaaatat cagtgcagtg cgtttaccac gtgaacccag
caatccagag aggttgaaag gttttggtta tgctgaattt gaggacctgg attccctgct
cagtgccctg agtctcaatg aagagtctct aggtaacagg agaattcgag tggacgttgc
tgatcaagca caggataaag acagggatga tcgttctttt ggccgtgata gaaatcggga
ttctgacaaa acagatacag actggagggc tcgtcctgct acagacagct ttgatgacta
cccacctaga agaggtgatg atagctttgg agacaagtat cgagatcgtt atgattcaga
ccggtatcgg gatgggtatc gggatgggta tcgggatggc ccacgccggg atatggatcg
atatggtggc cgggatcgct atgatgaccg aggcagcaga gactatgata gaggctatga
ttcccggata ggcagtggca gaagagcatt tggcagtggg tatcgcaggg atgatgacta
cagaggaggc ggggaccgct atgaagaccg atatgacaga cgggatgatc ggtcgtggag
ctccagagat gattactctc gggatgatta taggcgtgat gatagaggtc ccccccaaag
acccaaactg aatctaaagc ctcggagtac tcctaaggaa gatgattcct ctgctagtac
ctcccagtcc actcgagctg cttctatctt tggaggggca aagcctgttg acacagctgc
tagagaaaga gaagtagaag aacggctaca gaaggaacaa gagaagttgc agcgtcagct
ggatgagcca aaactagaac gacggcctcg ggagagacac ccaagctggc gaagtgaaga
aactcaggaa cgggaacggt cgaggacagg aagtgagtca tcacaaactg ggacctccac
cacatctagc agaagtaagt cagaccagga tgcacgaagg agagagagtg agaagtctct
agaaaatgaa acactcaata aggaggaaga ttgccactct ccaacttcta aacctcccaa
acctgatcag cccctaaagg taatgccagc ccctccacca aaggagaatg cttgggtgaa
gcgaagttct aaccctcctg ctcgatctca gagctcagac acagagcagc agtcccctac
aagtggtggg ggaaaagtag ctccagctca accatctgag gaaggaccag gaaggaaaga
tgaaaataaa gtagatggga tgaatgcccc aaaaggccaa actgggaact ctagccgtgg
tccaggcgac ggagggaaca gagaccactg gaaggagtca gataggaaag atggcaaaaa
ggatcaagac tccagatctg cacctgagcc aaagaaacct gaggaaaatc cagcttccaa
gttcagttct gcaagcaagt atgctgctct ctctgttgat ggtgaagatg aaaatgaggg
agaagattat gccgaataga cctctacatc ctgtgctttt ctcctagttt ctctccaccc
tggaacattc gagagcaaat caaaacctct atccagacaa gacaaaataa aactcaccat
ctcctgaaga cctttcttac ctttttttaa aaacaaaaaa tgaaattatt ttgcatgctg
ctgcagcctt taaagtattg aagtaactgg agaattgcca atacagccag agagaaaggg
actacagctt tttagaggaa aagttgtggt gcgttatgtc accatgcagt tgccagtgtg
attagtgcct aggggtctcc atttagcaga aatggtaatg acagtgatat aatgcctgga
acctggttgg gcagtagggg agggaggtag aaggaaaagt gtgagatttc taccttttag
tttttatcct attgtggcat atatgaattc tcaaacatta tctgaataaa ttttccactc
ttggaaaggt agatttagcc tcaagttgtt ctagtctcca ggaggctgcc agcccctcct
cttatttaat tctgagtttt gggggccagc ctagagggaa ttcctttttt ttttttaacc
ccccaggggg gtagttggga gtgagactat aggccataaa gaatgggact gcattggacc
aaaataaatg ggaaaatcgt ggtttgaaaa gaagcttttg ggaagtgatg agtcattttg
caccaggtaa taggggaaaa ttgtgtgacc tccagcaaac acatgaatgg ttatttcctg
gagccggaag cacttggggg tcgtggtaat tcccagtgtt ttctgtgtcc tagttttacc
ctttctaaac actgtccttt ttgaaagttt tgaatatatc cacattctat tgaaaccttg
aaactaaaaa tttagactct tatcatcatc ttaagttctt catgctactc ttaacctccc
aaaaagcagt atctaagtca catacatgat gtcttgggca ttttctcagc catggagaac
tctgaaagga agaatcgctg cttttctcaa gcaaatcggt ttcttgatgt cttttggttc
tccttgcctg ctcctgatgc ttggacccct tttattgatc agagtgctct agaataatgg
atggtcttgg atgatggata aatagggaca gggacagtta aattgggagc ctttcttaca
accttgatgg gatttttccc cccaagtttc cttctccact gaaatgccac actaatgctt
gttggattca tgaggtggcc agaccaatgt gttgttttgt tgttgttttt ttaagcttcc
cttgagagaa taaatggtaa tggagagaac tatttaacaa ggtcctggtt tctcttgcaa
cacagtagct aaacttgcct gcttttatat gcatttttgt agggatcagc ttggtagaca
gtattagcgg agaaacacct tgatcttggt ttgcaagccc ttctcccatc agtcctagat
taggccctgt tcagccatgc aggggtgttg gtttatgcgt gctgcagcag tgggcataat
gaatataatt tacccagtgg acaaaggtgt gtaccaagtg aatttaaata attggtgtgg
attggccagt agctaagaag tgggctttta aagagtattg aagattgaaa gggtttttct
ttctttttta aaaaagaaaa acaaactatt gattgtagat aatgaaaagc tagggtttgc
cctcttcatg tctactctcc ttccaaatag ttatatccaa aactgttttt ccctctcccc
taccttgtcc cccctattaa aatagaaaca gggattgatt aatgtcccgc tcctgaatac
atgtaaaatt tgtacaaaaa tatcttctat gaaaatgatt tgtaatctgt agacttatta
cctgggagat gtcttgatgt aaaatcccat cctttgggtt gtgggttttt tgttttctcc
aaataaatct gatctttaaa gttcattgta a aatcgtcggt ttctttctgg
acactgactt caaaaataaa ataggatatg aaaatgg

(SEQ ID NO:52), a different isoform of the RNA, or a nucleic acid having at least 80%, 82%, 85%, 86%, 88%, 90%, 92%, 94%, 95%, 97%, 98% or 99% nucleic acid sequence identity thereto, is detected.

In one embodiment, a product encoded by Homo sapiens MORF4 family-associated protein 1 (MRFAP1), mRNA NCBI Reference Sequence: NM_001030009.2, e.g.,

MRPLDIVELAEPEEVEVLEPEEDFEQFLLPVINEMREDIASLTR
EHGRAYLRNRSKLWEMDNMLIQIKTQVEASEESALNHLQNPGDAAEGRAAKRCEKAEE
KAKEIAKMAEMLVELVRRIEKSESS

(SEQ ID NO:53), a different isoform of the protein, or a polypeptide having at least 80%, 82%, 85%, 86%, 88%, 90%, 92%, 94%, 95%, 97%, 98% or 99% amino acid sequence identity thereto,
or a gene comprising or RNA corresponding to

attttgttcg ccgttactct gcgcgtaagt cgcttgtccg tggcttctct gagaagaaaa
gttgaaaaag ggtaaaagtt ttcaggaata ttcgggctct ctattgctaa gcatagcgag
tgtcggtttt ctctctccaa cagacatcgc tattgcggtt ccgaggcagt gggaagagat
gcggcccctg gacatcgtcg agctggcgga accggaggaa gtggaggtgc tggagcccga
ggaggatttc gagcagtttc tgctcccggt catcaacgag atgcgcgagg acatcgcgtc
gctgacgcgc gagcacgggc gggcgtacct gcggaaccgg agcaagctgt gggagatgga
caatatgctc atccagatca aaacgcaggt ggaggcctcg gaggagagcg ccctcaacca
cctccagaac ccgggcgacg cggccgaggg ccgggcggcc aagaggtgcg agaaggccga
ggagaaggcc aaggagattg cgaagatggc agagatgctg gtggagctgg tccggcggat
agagaagagc gagtcgtcgt gagcgcggtc ggcggtttcc agccaatgga ttctggtcaa
ctggtggaga ttggctgaca ccctggagaa gccgaaacca gagagccttt tgttttctct
tttttcctgt ctatgctctg tctcacttaa cactacgttt tctgctatgg tctgtggttg
atgacctcaa tatgagtttc gattgttaac gtgtttttgt ttgggaagta attttgtttg
aaaatgctct cacatacagg aattagggcc tagattgtaa gctcttgcag cagtcacatt
tgttcccggg ctttggtggt tatttctaaa tttttgaggt gctttgctat ttcttgtgtg
acctgatagc tccctggaac tttgggtctg tgtgtgacac atgagactca cagttggagt
tctccagctc tggaggtgct ggaagacgac cattaattct gaaggagctg tccatgcagc
aactactgaa gaaaggacca gacttcaacg gggagtgtgg atgggccgac ctggctggga
ctcgtgaatc tggagaagag ctggagaatg gatagtattg tctgtatttg gagactttaa
tttctgtgtg agaccaaagg aggagagatg tgttttgttc aaaatttaaa tttgttgtgg
tacactatct tatgtaacct gtctggtgag tttgtttgga caacctaact cagctttatt
tgacatggaa cctaaaatag aagataagat cttgatattc tgtacaagtt gatgtaatac
cctgatgcgt tttagaggac ttggcataaa atgaaagatt ggcaaaggcc cttgaggggc
ttggggatga cagtatggaa ctgtctgcat tggaccctaa actggactag aagaggcatc
ttcaaggttc atacgttgtc cagctgtaag ttcatttgag tagcagacct aacaaatatt
tgaggtcaga accctaccat gttaaaacaa acaaaaactt accatgttaa taaaagtatt
catttgcttg aaaa

(SEQ ID NO:54), a different isoform of the RNA, or a nucleic acid having at least 80%, 82%, 85%, 86%, 88%, 90%, 92%, 94%, 95%, 97%, 98% or 99% nucleic acid sequence identity thereto, is detected.

In one embodiment, a product encoded by Homo sapiens Polyadenylate-binding protein-interacting protein 1 (PAIP1), mRNA NCBI Reference Sequence: NM_006451.5, e.g.,

MSDGFDRAPGAGRGRSRGLGRGGGGPEGGGFPNGAGPAERARHQ
PPQPKAPGFLQPPPLRQPRTTPPPGAQCEVPASPQRPSRPGALPEQTRPLRAPPSSQD
KIPQQNSESAMAKPQVVVAPVLMSKLSVNAPEFYPSGYSSSYTESYEDGCEDYPTLSE
YVQDFLNHLTEQPGSFETEIEQFAETLNGCVTTDDALQELVELIYQQATSIPNFSYMG
ARLCNYLSHHLTISPQSGNFRQLLLQRCRTEYEVKDQAAKGDEVTRKRFHAFVLFLGE
LYLNLEIKGTNGQVTRADILQVGLRELLNALFSNPMDDNLICAVKLLKLTGSVLEDAW
KEKGKMDMEEIIQRIENVVLDANCSRDVKQMLLKLVELRSSNWGRVHATSTYREATPE
NDPNYFMNEPTFYTSDGVPFTAADPDYQEKYQELLEREDFFPDYEENGTDLSGAGDPY
LDDIDDEMDPEIEEAYEKFCLESERKRKQ

(SEQ ID NO:55), a different isoform of the protein, or a polypeptide having at least 80%, 82%, 85%, 86%, 88%, 90%, 92%, 94%, 95%, 97%, 98% or 99% amino acid sequence identity thereto,
or a gene comprising or RNA corresponding to

ggaaagccga gggtagccga gcggggcggg cgctctggag cggcgggtgc tcgggctgcc
gtccgctccg ccagaagcac cgagcagccg agccggggcc cgccgccctc ctcctccatg
aggcccgagt gaggcgcggc ggctatagcc gacccgcggc gccttccccc cgcgtcctat
cgcgagcgca gcggcagcgg cccctggagg aggaggcgga ggaggaggag catgtcggac
ggtttcgatc gggccccagg tgctggtcgg ggccggagcc ggggcctggg ccgcggaggg
ggcgggcctg agggcggcgg tttcccgaac ggagcggggc ctgctgagcg ggcgcggcac
cagccgccgc aacccaaagc cccgggcttc ctgcagccac cgccgctgcg ccagcccagg
acgaccccgc cgccaggggc ccagtgcgag gtccccgcca gcccccagcg gccttcccgg
cccggggcgc tcccagagca aacgaggccc ctgagagctc cacctagttc acaggataaa
atcccacagc agaactcgga gtcagcaatg gctaagcccc aggtggttgt agctcctgta
ttaatgtcta agctgtctgt gaatgcccct gaattttacc cttcaggtta ttcttccagt
tacacagaat cctatgagga tggttgtgag gattatccta ctctatcaga atatgttcag
gattttttga atcatcttac agagcagcct ggcagttttg aaactgaaat tgaacagttt
gcagagaccc tgaatggttg tgttacaaca gatgatgctt tgcaagaact tgtggaactc
atctatcaac aggccacatc tatcccaaat ttctcttata tgggagctcg cctgtgtaat
tacctgtccc atcatctgac aattagccca cagagtggca acttccgcca attgctactt
caaagatgtc ggactgaata tgaagttaaa gatcaagctg caaaagggga tgaagttact
cgaaaacgat ttcatgcatt tgtactcttt ctgggagaac tttatcttaa cctggagatc
aagggaacaa atggacaggt tacaagagca gatattcttc aggttggtct tcgagaattg
ctgaatgccc tgttttctaa tcctatggat gacaatttaa tttgtgcagt aaaattgtta
aagttgacag gatcagtttt ggaagatgct tggaaggaaa aaggaaagat ggatatggaa
gaaattattc agagaattga aaacgttgtc ctagatgcaa actgcagtag agatgtaaaa
cagatgctct tgaagcttgt agaactccgg tcaagtaact ggggcagagt ccatgcaact
tcaacatata gagaagcaac accagaaaat gatcctaact actttatgaa tgaaccaaca
ttttatacat ctgatggtgt tcctttcact gcagctgatc cagattacca agagaaatac
caagaattac ttgaaagaga ggactttttt ccagattatg aagaaaatgg aacagattta
tccggggctg gtgatccata cttggatgat attgatgatg agatggaccc agagatagaa
gaagcttatg aaaagttttg tttggaatca gagcgtaagc gaaaacagta aagttaaatt
tcagcatatc agttttataa agcagtttag gtatggtgat ttagcagaac acaagagagc
aagaaaatgt cacatctata ccaaattaag gatgttgagt tatgttacta atgtatgcaa
ctttaatttt gtttaacact atctgccaaa ataaacttta ttccctataa cttaaaatgt
gtatatatat ataatagttt attatgtaca gttaattcta ctgttttggc tgcaataaaa
tcgattttga aataaatgaa atgttgaaaa ttttgctagt tggttagatg cttatccttt
aaattctact tttcttgagg ggaaaaagtc ttcttctgga aatacatatt actgcaaaaa
tgtagcatcc ttttttaggt aggagtatta tagctttcat tttagtttga catttagtgt
cccaatgaat tgaatttcaa atatgaatca taatcttgaa aatctttagc actaaagtct
tggaatatat caacaactga tttacatatg cagatgctat ttgataccaa gggcttttta
aatgtcatgg gggggaaaaa cccaacttgg tgaactccca gctaaacaac caagacttca
ctgaagattt attccaattc tagaattgtt cttttttatt tttatttttt caactgacta
acttcattac cttaaaggct agaacattat tctgctttat ttatatggct ttctcacttt
tattttgtag catgggttgc atcgactttt ttactagaga attttactag atatttgtca
ttcaagtttt catctgcttt ataattgata caccttgagg gtcacttttc taatactttt
actataatgt ggtaccacct cagccctaat aaataatatt tttacctaat gtcaaatctt
tttccagcta actaaaaact gtgtacaaaa ggattgcttg taaatatgca tgtaaatagt
tctgttaata acccactgtt ttacatttgg tacatctgtg tctgctaata cagttagctt
tctcactttt ctgcttgttt gttcagtctg aattaaaatt agactttgaa aataaagctt
aaatagttgt ttcctctaaa

(SEQ ID NO:56), a different isoform of the RNA, or a nucleic acid having at least 80%, 82%, 85%, 86%, 88%, 90%, 92%, 94%, 95%, 97%, 98% or 99% nucleic acid sequence identity thereto, is detected.

In one embodiment, a product encoded by Homo sapiens Pancreatic Progenitor Cell Differentiation and Proliferation Factor (PPDPF), mRNA NCBI Reference Sequence: Q9H3Y8⋅PPDPF_HUMAN, e.g.,

MAAIPSSGSLVATHDYYRRRLGSTSSNSSCSSTECPGEAIPHPPGLPKADPGHWWASFFFGKSTLPF
MATVLESAEHSEPPQASSSMTACGLARDAPRKQPGGQSSTASAGPPS

(SEQ ID NO:57), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a product encoded by Homo sapiens Ribosomal modification protein rimK like family member B (RIMKLB), mRNA NCBI Reference Sequence: NM_001030009.2, e.g.,

MCSSVAAKLWELTDRRIREDYPQKEILRALKAKCCEEELDFRAV
VMDEVVLTIEQGNLGLRINGELITAYPQVVVVRVPTPWVQSDSDITVLRHLEKMGCRL
MNRPQAILNCVNKFWTFQELAGHGVPLPDTFSYGGHENFAKMIDEAEVLEFPMVVKNT
RGHRGKAVFLARDKHHLADLSHLIRHEAPYLFQKYVKESHGRDVRVIVVGGRVVGTML
RCSTDGRMQSNCSLGGVGMMCSLSEQGKQLAIQVSNILGMDVCGIDLLMKDDGSFCVC
EANANVGFIAFDKACNLDVAGIIADYAASLLPSGRLTRRMSLLSVVSTASETSEPELG
PPASTAVDNMSASSSSVDSDPESTERELLTKLPGGLENMNQLLANEIKLLVD

(SEQ ID NO:58), a different isoform of the protein, or a polypeptide having at least 80%, 82%, 85%, 86%, 88%, 90%, 92%, 94%, 95%, 97%, 98% or 99% amino acid sequence identity thereto,
or a gene comprising or RNA corresponding to

gcggcaggcg agtgagggaa cgaggagcgg ccgggtgtga gtgtgtggga gtgagagtgt
gtgggagtgg ggtgagggag aagctgacgg gacgcgaggc tgtgagaaac tgggcgagtg
tgcgaggacg cccggccagc ctgcgggagc cgcagtcggc ggaggagaaa ggaggcggct
cccggtatcc cgaccccctc cccctcctct ccttccccca cttccagccg cccggcggcc
cgcgcttcct cgaaggcccc agcccggctc agtcggccga gagcgaggga ggagcccccc
gacccaggtg ctggagtctg cttgtcaggg aggggggcag tctgtgattc tgagaacaga
gccaagaagg ggaacagcaa attcagtcac agacaatcct ccactcggtc aagagccact
tttctcttcc tgccttgccc ccccgcaggg ggtaaggaac tgagcgttta atctttagcc
ggttggctac cagctaaaat tctacttatc ttagtttcta gtggatagct ttcttatttt
gcccatgttt tcttagaatc cctgtttaat atacttttgt cagtagtagt atctaggagt
agcagggaga gtgacaataa attagcccct tcttttttcc cttgtcattc aggccccttt
tcctctccag agggaaatta ccagtaaact cttctaaatc ttccacccct tctcagtcat
actgtgaaga aacacactaa agtggacatt atttgaccag tgaacacgaa cccagcttca
ggcattggtt tgttgtggca catggagaaa catctctttt aaaatatctc ccaattaccc
ttttcacaat ttgtatccac ctaggatttg ctgctggggt aagtcactag atttatttct
caaagctccc ctctctatga gctgaaagac tgaccaacca tgaacactag taggggatgg
ggaaagggga cagagcagag ccagttgttc cacactttgg gaagcaggag tagcttttat
catcttcctc tggggagcag gcatagagac ataaactgag tgaaaatggg tggaggaaga
acttctatac ccacgaacaa catgtgaaga gagagaacca aacataaagt aaggagggta
gacgttacat ccaagaggaa ataatccagg caaggaagca caagctgatc aagatgtgta
gttctgtggc tgccaagttg tggtttttga cagatcgtcg catcagggaa gactatcctc
aaaaagagat tttacgagca ttgaaggcca aatgttgtga ggaggaactg gactttaggg
ctgtggtgat ggatgaggtg gtgctgacaa tcgagcaagg aaacctgggt ctgcggatca
atggagagct aatcactgcc tacccacaag tggtggtagt cagagtacca accccttggg
tgcaaagtga tagtgacatc actgttttgc gccatctaga gaagatggga tgtcggttaa
tgaaccgacc tcaagccatc ctgaactgcg ttaataagtt ctggacattt caagagttgg
ctggccatgg tgttcctctg ccggatactt tctcttatgg tggccacgaa aattttgcta
aaatgattga tgaggctgaa gttctggagt tcccaatggt agtaaagaat acgcggggtc
acagaggtaa agctgttttc ttggctcgag ataagcacca tttggctgat ctaagccatc
ttattcgcca tgaagcgcca tacctgttcc agaagtatgt taaagagtct catggacggg
atgtacgtgt cattgtcgtg ggaggccgtg tggttggcac catgttacgt tgttcaacag
atgggagaat gcaaagcaac tgctcattag gtggtgtggg gatgatgtgc tcattgagtg
aacaagggaa gcagctagct atccaggtgt ctaatatcct ggggatggat gtgtgtggca
ttgatctgct gatgaaagat gacggctcct tctgcgtctg tgaggccaat gcaaatgtag
gtttcatcgc ctttgataag gcttgtaatc tagatgtagc tggtatcata gcagactatg
ccgcctccct tctaccctct ggccggctca cccggcgtat gtccctgctc tccgtggtgt
ccactgccag tgagactagt gagccggagc tgggtccccc agccagcact gctgttgaca
acatgagtgc aagttccagc tctgttgaca gcgaccctga aagcacggag cgagagctgc
tcaccaagct cccagggggc ctgttcaaca tgaaccagct gctagccaat gaaatcaaac
tactggtgga ctgactccac tggtaattaa ccaacaaaac ccttgtaaaa ctttctttct
tcttttctat ttttaaaacc aacttgcaat gctgttcatg gaggatgctc aggaagatga
gagaaaatta gtaggattag ttggagagag tgggagatag atgagacctc tgctagtaag
atgttacttt catttacaaa tcctacaaat agagaggcag aataggtggg gtatagaaaa
atgtcaggct ctcatagtta cccttttaaa ttgctaaaaa atgtgtatgc tcataggcca
tgaggaacaa atactttttt tttttcatgg tcccttgctt ttgtttttgt acaaaaaaaa
atggttttgc tacaaatatc caagtagcat aacttcacat tgtgttggaa gatttgtcat
cagtgaggaa aacatctgca taaattacag gaatttttgt attatacagc tctgaaaatt
ctgccatttc cttattaact agcagcttta gtttgtagtt tatgaaatct tgaggggctc
ttttactggg atttcttatt tttttgtttt ttcccgctta atttggtggg aggtcaaatt
gaatataacc caataaaggc ttcttaatga caaaattggc atgtttgcat gatgaaatgg
aaatgaacag tattgcaatg tccggtatac aaaataacat taattcaatg tagataaaat
tacactagtt taaaatatgt gcattcactt gtatttgtta gtgttttagt cttttttgaa
agatgtgctc tgttaatgtt gctttttttt ttttttttaa tacatgctag tctaacattt
cctgctctat gcctgcatct ttaacaatgg ccaaagtgaa gaaaatgcta ccttttttgt
taacaagaca ctgacttgaa acatgtacat ttaaagcctt ttattttttc cctttttgtt
ttggtagttg ggcatttaaa taaggacaag gaaaaatatt tttgggggca aatcaagagc
ctatgagttc taagtataaa gctgaagtga tttcgaatgc cagcgttata tatttgcatt
tttcacattt tacgagggag tatatgtgta tgtgtgtgca cgcatgcatg tgtatgtgtt
ttgctttttg tttccatcaa ctaatcaaaa aggataattt agaaaatgga gcatgatggg
aaacagagtt tttgacttta aaaaacagat gagttgtttt cataagtaga ctccactggg
gtagaggtat tcaccttaaa acatagggtg agtagatgct tttttaggcc tttttgtgta
tatgtacgtt gtttgttttt ttccttttgt ttctagcctg ttcagtgtac agtttattca
aggctacatg cttttcttta atgcttctgg ctatgcattt tctcttttta catataggat
ttgggattgg gggtgggttg gatgtttttg tttggggact tatttagtag tattgagtct
cttatagccc tactcttaag ccttcaatac tgtccactct ttatattcct ttacttgcag
aatttataaa agcccccaaa ctgcatataa tatgagcctt taaaacatgg gtaaaactaa
tcccattgat gggtttggat ggtatgttaa gaaatggaga tgctgcagag cccaacgtaa
ttttttaaac agcaagtttt ccatctccct acgaatcctc tgaagctttt acccaagccc
tttcttgcct ctccagtgct attttccttc agatggacct taaacataat ttcttggaca
ctactagaga gacttcgagg caataataaa agatcagtat taaccagcta taacagaggt
ttgatcatgc ttacttgtac agtttttccc ccgttttaaa aaggaatgta ataaaatttg
ttttttccat agaattaaat aatattaaaa ttgagtgaaa ggttgattgt tgatgaatag
aatagtacct ctcatctgtg cagtgtctca tttcacctca gagaaaagga tacataagag
gagtttgtaa tttatcttag gatattctaa ttgcatttaa aagaacttat cttgcgcagg
gtaaatgggg gactcacata catatattaa tacctctgac tcattaacag aaagaaatac
ttggtacttc tttcgctgaa tgaccatact gtggaggatg catactattt ggtatagaga
aataaatgag gaagaaagaa ctgcttaatt aaattatcat tcatatgttc atatagagac
catctggttg ccatgtgtat tatgacacat acactttgaa tagttacata tcacaagtat
gtagttcatg tttgtgttgg tggggtaagg catcaggaaa aatgtagtta gtcttttctt
aacttatacc aaattaacca actatattat aggaaatatg tgaaattagt tcattagctt
tattcactat tatgcattca catgatatta aaacgtacac tcacatgtta gaatgaaaag
agcagtagtt atcttagatt ttaaaaacat ggatatcttc ttgaattcct tcaagattga
ggtagagaat aagagcaaat cattctggaa gtaccttaag gaaacaaaca gcagcagata
tttaggttaa acttattttc ataattgttt aataactttt gtataatctt cattgctatt
atgagagaga atgtatatat caaatatgtg taatgataaa atctgaattg taaaattttt
gtatattgtt aaaattgtaa ttctaaattg tatttcaaaa atgattattt ctgatattgt
ttttatgtca cccatgatga aaactggact ttatatatct aaacatacaa gtatgaacta
ttctatttaa aatttttaat agtttttttc ttttttggtg cctataattg attggtcatt
tctgctggct tttctccaat gaacattgaa atcttcctgt atatgttacc aataagaaaa
ctaccctgga acagtagaaa aacccaacaa gagacttggc attcatcaag cacattatca
gactttgaga acatattgaa ggcattgact ttgaaaatca tctctttttc tcaagaagaa
agcaatggag aagcaaattt gtttcattca gtgaatcccc agtttggggc ttgtggggct
tagagacatt gtgaaatcaa atcttgtgtt atacttttct cctggctcac tttttttgag
aaggtttatg ggctatttgg ctggtgagac acgatcccct cctaagaaaa tgtaggtgct
cagacaggta accactgctg ctactgtttt tatttgtttg tttgttcaat tttatttaag
atttgttttt gttgtactag gattttaaaa aatgtaatat attgcaggat ttataaccag
g

(SEQ ID NO:59), a different isoform of the RNA, or a nucleic acid having at least 80%, 82%, 85%, 86%, 88%, 90%, 92%, 94%, 95%, 97%, 98% or 99% nucleic acid sequence identity thereto, is detected.

In one embodiment, a product encoded by Homo sapiens Signal Peptidase Complex Subunit 2 (SPCS2), Reference Sequence: Q15005⋅SPCS2_HUMAN, e.g.,

MAAAAVQGGRSGGSGGCSGAGGASNCGTGSGRSGLLDKWKIDDKPVKIDKWDGSAVKNSLDDSAKKV
LLEKYKYVENFGLIDGRLTICTISCFFAIVALIWDYMHPFPESKPVLALCVISYFVMMGILTIYTSY
KEKSIFLVAHRKDPTGMDPDDIWQLSSSLKRFDDKYTLKLTFISGRTKQQREAEFTKSIAKFFDHSG
TLVMDAYEPEISRLHDSLAIERKIK

(SEQ ID NO:60), a different isoform of the protein, or a polypeptide having at least 80%, 82%, 85%, 86%, 88%, 90%, 92%, 94%, 95%, 97%, 98% or 99% amino acid sequence identity thereto.

In one embodiment, a product encoded by Homo sapiens Protein sprouty homolog 4 (SPRY4), mRNA NCBI Reference Sequence: NM_030964.5, e.g.,

MLSPLPTGPLEACFSVQSRTSSPMEPPIPQSAPLTPNSVMVQPL
LDSRMSHSRLQHPLTILPIDQVKTSHVENDYIDNPSLALTTGPKRTRGGAPELAPTPA
RCDQDVTHHWISFSGRPSSVSSSSSTSSDQRLLDHMAPPPVADQASPRAVRIQPKVVH
CQPLDLKGPAVPPELDKHFLLCEACGKCKCKECASPRTLPSCWVCNQECLCSAQTLVN
YGTCMCLVQGIFYHCTNEDDEGSCADHPCSCSRSNCCARWSFMGALSVVLPCLLCYLP
ATGCVKLAQRGYDRLRRPGCRCKHTNSVICKAASGDAKTSRPDKPF

(SEQ ID NO:61), a different isoform of the protein, or a polypeptide having at least 80%, 82%, 85%, 86%, 88%, 90%, 92%, 94%, 95%, 97%, 98% or 99% amino acid sequence identity thereto,
or a gene comprising or RNA corresponding to

gcaacatcgc cgcggaggta gcgagctgag ctgacagcgc ggagctggcg ctgtggagcg
cagggagcct tgccggttcc tccgaccggc gtctgcgagt acagcggcgg ctaacctgcc
ccggcttcag gatttacaca gacgtggggc gatgcttgtg accctgcagc tcctcaaacc
agcctgtatt gagcggtttg cagcctgatg ctcagccccc tccccacagg gcccctagaa
gcctgtttct ccgtacagtc caggacctcc agccccatgg agcccccgat cccacagagc
gcccccttga ctcccaactc agtcatggtc cagccccttc ttgacagccg gatgtcccac
agccggctcc agcacccact caccatccta cccattgacc aggtgaagac cagccatgtg
gagaatgact acatagacaa ccctagcctg gccctgacca ccggcccaaa gcggacccgg
ggcggggccc cagagctggc cccgacgccc gcccgctgtg accaggatgt cacccaccat
tggatctcct tcagcgggcg ccccagctct gtgagcagca gcagcagcac atcctctgac
caacggctct tagaccacat ggcaccacca cccgtggctg accaggcctc accaagggct
gtgcgcatcc agcccaaggt ggtccactgc cagccgctgg acctcaaggg cccggcggtc
ccacccgagc tggacaagca cttcttgctg tgcgaggcct gtgggaagtg taaatgcaag
gagtgtgcat ccccccggac gttgccttcc tgctgggtct gcaaccagga gtgcctgtgc
tcagcccaga ctctggtcaa ctatggcacg tgcatgtgtt tggtgcaggg catcttctac
cactgcacga atgaggacga tgagggctcc tgcgctgacc acccctgctc ctgctcccgc
tccaactgct gcgcccgctg gtccttcatg ggtgctctct ccgtggtgct gccctgcctg
ctctgctacc tgcctgccac cggctgcgtg aagctggccc agcgtggcta cgaccgtctg
cgccgccctg gttgccgctg caagcacacg aacagcgtca tctgcaaagc agccagcggg
gatgccaaga ccagcaggcc cgacaagcct ttctgacagt ttgtgtcgaa gccccagtgc
tctgcctgga aacctggttc tcttctgaca tctaagaaga ctgcagcaag gtcagaggtt
ttagcctcct gaggctgacc ttgctagtct gcccactccc tacccccagc ttcggaaaat
acagagacca ccaccacgta ccctgtattc cccaaggtga tgaagaagca ctttggggct
ttttttcagg gtcctgaaac tttgtgtcaa acagacaatg caggggcagg gtgtggtttg
gggggaaatt tttctttttc agaagacaga acacagatgt ggacacatat ccggaaactg
cagctgcttg aatgccttcc cagcccctcc ttctccctcc ctccctccgc cccccccccc
ttcctctttt ccattgtctt tggctctcac aggagctagc tgcctgggag gaattgttaa
ctgagtacca gggtaccttt aaagaagacc cttggagtct tctatacctt cttctccttc
cccatctcac tccaccccac tttgtccctg atgtcttggg gaaggtgtag aacaccctag
cagttcctat tgtatatact tgggagccac tgagaacaga ggacggccag tgagtccaag
cctcgttcct ccttctgcct ccccggagcc acaggatgga tttaggagcc actgctcagt
gcacttctcc cttccaactg catcaactaa ctctcggggg tgttctgctc accacaccgt
ccttcggttc ttactgagtc acagactcgc ctgcccacta cgtgtcctgg gttctctcta
ctcagatccc ttccagaaac tttatatggg tagaggaagc cagggcggca aatgcgagac
caaatatcat tttgccaatg agtctgaggc tgtggtctct ggatccagtc attatgtttt
tatagaataa ttaaaccgga tgctaacggt gttttaaaaa ataataataa aacaacttgt
ttccttttgg ccacccccag gaagggctga tttcaaaatc tgggggcgag caacctcaag
gaacacaatt tccctcccta tcaacaagag gattttaaca gcaaagaaga gaggcagcac
ctcccattgg cagaatgacc gctgagccag gctgggtttg ggtttcttct cttctgattc
tgctgctcac tgtcatagcc ttttgtgtat agtgatgtgt ctgtatcttt aatgtaaata
gagagatgat gaaaaaagag tctattttag tgttaggaag ccccagcagg ggagtcggaa
gagcttggaa gagctgggga gagggtaggg gaaaggtttt tccaggggcc actgggtttg
agccctgctt ctgtgcacag ccacaccacc ctctcccgac agccctcaaa gacgtagcaa
ctctttctct caaggtgcta aaggactcag aaggtgcagc acgtccagtg ggtaggtact
tgttgcatgc aaaagctgta gtgtatctgg tccttcctcc ccagcttttg tgtggggttc
ttgctttgtg tggtattttg ttttcccctc taatgagagg gcatggcctg agtcagaaga
gctaccccag gtgaaactgg aagtgcatga ggcagagcgt ccgtagcatt tccagtttgt
tctgtatagg aacagaggtg cctccgggaa ggaggcagcg aggtaggtag ctatgatagg
cacctaatgc ttctcaagga cttatttttt ccttcttgaa gactagtagt aacatcttat
gatttagagt aagttgattg taaccatagg tatttattga ttggaggaag ggagggtcat
attattttcg gctttattta tgtaacattt gctagcttgt aaaaggcgaa tgtgaaatat
tgcatctgca ttttccaagg ctgattcgtg tagctaccct tgccacagtt gtgacggatg
tatggatgtt cttgaacatt tcagaaggag tggtagaaaa aaacacacat tcagccaacc
acttatatga attgaatgta tcagaagtgt actgaaggga ctggagatgg ttttcctcag
atgagggggc cccaaaattg atagtgcaca tctgcacgct ttctgcgagg cctcagaact
tcccagggcc cctccctcaa attgtctcca tgggaaactt gacccagtgg caagttgcac
tttggtgatc ttggtggtct acacacccgt tctgtggaga gtcgatttac ataagctgtg
tatacacaca cacacacaca cacacacacc cctaccccac actgactgtc taccgacaaa
gaccctattt cctggcaaac ggcctcctga accctgactt tttgtgtaca tacttgtaaa
cacggatttt tctgggtttt ggtttgcttt ttcctttttt ccccctgccc ctgttctagc
ttgttcttct tggtttgctt tcaacctgct tgatggatgt ctgcagagtg ctctctaaga
gtccacctca gtgcctcgtg tgctcagtgg tcatgggaaa ggagcgaagg aaccatcctt
ggttctccca gcttggttgt gtagcaatcc ctcagcattg tttttctcag cttcttggca
aaaattaaaa caacaacaac aacaacaaca acaacaacaa acagaaggat aaactggctt
gcctgtggac cctccccggc tctggggcca gtcgagagcc actgagggac ccagcactca
gagacacaac acacatgtgt agctgcttct ggctgagtgt gtttcctgtc accaatggcc
tgtttggctg gacgatgcct cggcttgacc ttttttgaaa agtgctggtt agttcccgcc
cctggtaaac ctggggtagg tgggggttct gtcttaactc gaggggcacc tgggatccag
gacgcttcta gggggctctg gctgcccgtg ttaatgaagg acagcgcttc cgcgagcacc
ctgggaactg ggtcttgggt agcaaagccc tcccagagaa aagattgggc acaactaagg
ctttcctgag caggaagggg gtgaagacca atcccttcct ttggtccttt ggtacgcacc
ccctcagagc tgagatggaa gacatggcta gttcttttca gccttgtgga gcctgtcagt
cgccatcata cctcgagtga ggcccagcta gataatgact tgtccaagat ggcacacgtg
gaaagttgat ctgcaccaga acccggatga ctgtcacctt gaagcatcct gttctccttc
tgtgctgtcc caggaagtgt ctggcgggcg tgggcagcac agctctacac tgtacgattc
actagggcat cctgcgagcc tcactagcct tctggttcat gcctttgaca agcatttttg
tgccccctct gcttactgtg acagtcgatg atgaatcttg cgttgccatt ttctgctgtg
ggtaactgcg tgcagtgtct tgccttgctt tctcttctta ctgtcccaca gcttggtttc
atgttacaaa cagaaaagct cgaggctccc accccgccac atcccaactt catttccccc
tcactgtagc ccatttccac cccaccacaa agttgccaca ggttttcttt gtatagaata
tttattttga agctctattt taatagtatt tattttagaa agtctactat tgtaagagtt
cttctgtttg tgaagaaaaa aacaagttaa aaactgaatg tactgattta gaaaatatat
ataaatatat attgttaaat atacacggga ctgcc

(SEQ ID NO:62), a different isoform of the RNA, or a nucleic acid having at least 80%, 82%, 85%, 86%, 88%, 90%, 92%, 94%, 95%, 97%, 98% or 99% nucleic acid sequence identity thereto, is detected.

### Exemplary Therapies

For mammals with an increased risk of or having MBM, e.g., based on expression for the genes disclosed herein, those mammals may be treated with various immunotherapies, targeted therapies and/or chemotherapies. Immunotherapies include but are not limited to talimogene laherparepvec (T-VEC), aldesleukin, peginterferon Alfa-2b, high-dose interferon alfa-2b, pembrolizumab, nivolumab, ipilimumab, or a combined nivolumab and ipilimumab Regimen. Targeted therapies include but are not limited to vemurafenib, trametinib, dabrafenib, a combined trametinib and dabrafenib regimen, a combined encorafenib and binimetinib, or a combined cobimetinib and vemurafenib Regimen. An exemplary chemotherapy includes but is not limited to dacarbazine. In one embodiment, immunotherapies include but are not limited to pembrolizumab (anti-PD-1 antibody) plus bevacizumab (anti-angiogenic); pembrolizumab, nivolumab (anti-PD-1 inhibitor), fotemustine (alkylating agent) fotemustine and ipilimumab (anti-CTLA-4 inhibitor), ipilimumab and nivolumab, or nivolumab plus ipilimumab followed by nivolumab monotherapy. In one embodiment, targeted therapies include but are not limited to dabrafenib (BRAF inhibitor) plus trametinib (MEK1/2 inhibitor), buparlisib (pan-PI3K inhibitor), abemaciclib (CDK4/6 inhibitor), WP1066 (STAT3 pathway inhibitor), dabrafenib (BRAF inhibitor) plus trametinib (MEK inhibitor), vemurafenib (BRAF inhibitor) plus cobimetinib (MEK1/2 inhibitor). In one embodiment radiation plus systemic therapy includes but is not limited to dabrafenib (BRAF inhibitor) plus SRS, nivolumab (anti-PD1 antibody) plus SRS, pembrolizumab (anti-PD1 antibody) plus SRS, ipilimumab (anti-CTLA-4 antibody) plus SRS, ipilimumab (anti-CTLA-4 antibody) plus WBRT, or Ipilimumab (anti-CTLA4 antibody) plus WBRT.

The invention will be described by the following non-limiting example.

### Example

Provided herein is evidence of RPL/RPS gene signature driving melanoma brain metastasis. Complex multilevel approach was performed to identify MBM signature and confirm its relevance to clinical settings. An MRI CTC-derived MBM mouse xenograft was established to monitor MBM spatial and temporal development and progression.

### Materials and Methods

### Patient Blood Collection and Processing

Patients diagnosed with primary or metastatic melanoma were enrolled according to protocols approved by the Institutional Review Board at UNM Health Sciences Center (UNM-HSC), Albuquerque, NM. All patients' blood samples were collected after receiving informed written consent, according to the principles of Declaration of Helsinki. Clinical details of each patient included in the study are provided in Table 1. Peripheral blood (12-18 mL) was collected either in CellSave (Menarini Silicon Biosystems, Inc.), or in sodium-ethylenediamine tetraacetic acid (EDTA) tubes under aseptic conditions. Blood collection was performed at the middle of vein puncture as part of patients' routine clinical care. Following blood collection, samples were sent immediately to the laboratory for isolation and analysis of CTCs. All blood specimens were analyzed within 24 hours following blood draw.

TABLE 1
Patients' clinical parameters

| PatientID | Gender | Age | Stage | Mutation Status | Metastatic Site | Treatment |
|---|---|---|---|---|---|---|
| Primary patient 1 | Male | 56 | pT1b NX | Unknown | None | None |
| Primary patient 2 | Male | 61 | pT3b NX | Unknown | None | None |
| Primary patient 3 | Male | 51 | pT2b III | Unknown | None | None |
| MBM patient 1 | | 50 | IV | GNA11, SF381, MYC ampl | Brain, liver | None |
| MBM patient 2 | Male | 58 | IV | BRAF negative | Brain, lung | Nivolumab |
| no MBM patient 1 | Male | 73 | T2b N2a III-B | ATM Q218, AXL R368Q, AXL | Lung | Ipilumumab, |
| | | | | R295W, CDKN2A R80, CSF1R | | Nivolumab |
| | | | | W58, GRM3 S154F, GRM3 G18E | | |
| no MBM patient 2 | | 46 | IV | BRAF V600E | Lung | Nivolumab |

Demographics and clinical-pathological characteristics of melanoma patients of this study. Clinical parameters of patients include gender, age, stage, mutation status, metastatic site, and treatment.

### CellSearch CTC Enumeration

CTCs positive for the human melanoma biomarker Mel-A (Mel-A+ CTCs) were captured and quantified by the CellSearch platform (Menarini Silicon Biosystems, Inc.), following manufacturer's guidelines. Samples (7.5 mL) were processed using CellTracks and the CellSearch melanoma CTC kit. CellSearch-captured CTCs are defined as MEL-PE+/DAPI+/CD45− cells (Vishnoi et al., 2018; Sprouse et al., 2019). Peripheral blood (7.5 mL) from healthy donors was used as negative control and subjected to the same process. In addition, the human melanoma CTC-derived clonal lines (70W-SM3 cells) were spiked at different concentrations in 7.5 mL of healthy donor blood as positive control. The automated CellBrowser software was used to visualize and quantify CellSearch melanoma CTCs.

### Peripheral Blood Mononuclear Cell Isolation and CTC Enrichment by FACS

Peripheral blood mononuclear cells (PBMC) were isolated by an established procedure (Vishnoi et al., 2018; Boral et al., 2017). Briefly, patients' blood was lysed with red blood cell lysis buffer (BioLegend, catalog no. 420302), and washed twice with PBS with 5 mmol/L EDTA (USB, catalog no. 15694). PBMCs were isolated and quantified by the Countess II cell counter (Thermo Fisher Scientific). Following cell blocking with Fc block (BioLegend, catalog no. 422302), PBMCs were stained for fluorescence labeling with FITC-CD45 (BioLegend, catalog no. 304038), FITC-CD34 (BioLegend, catalog no. 343504), FITC-CD73 (BioLegend, catalog no. 344016), FITC-CD90 (BioLegend, catalog no. 328108), FITC-CD105 (BioLegend, catalog no. 323204), Pacific Blue-conjugated CD235 (BioLegend, catalog no. 306612). Processed cells were then sorted using an iCyt SY3200 cell sorter (Sony Inc.) to separate Lineage-negative (Lin−) and Lineage-positive (Lin+) cell populations. FITC-positive cells were sorted into the Lin+ fraction, while the Lin− fraction consisted of cells negative for all fluorescent biomarkers indicative of normal cell lineage. Briefly, FACS gating employed the depletion of dead cells (DAPI−), followed by the isolation and elimination of leukocytes (CD45⁺), erythrocytes (CD235⁺), endothelial cells (CD34⁺), and mesenchymal stromal cells (CD73⁺/CD90⁺/CD105⁺ (Vishnoi et al., 2018; Sprouse et al., 2019; Boral et al., 2017)). CD235-positive cells were eliminated from downstream analysis. Data generated by FACS were analyzed by FlowJo V10 program, as described previously (Vishnoi et al., 2018; Boral et al., 2017)).

### RNA Sequencing

RNA was isolated from Lin− and Lin+ fractions (25-50×10³ cells, respectively) after FACS. RNA extraction was performed using a miRNA Isolation kit (Qiagen Inc., catalog no. 74004). RNA from matching Lin− and Lin+ fractions were compared with RNA from PBMCs of healthy donors (negative controls). RNA analysis, cDNA amplification, and library preparation were performed using the human microarray platform (SMARTer Universal Low Input RNA kit for sequencing (Clontech, catalog no. 634946). The Ion Plus Fragment Library kit (Thermo Fisher Scientific, catalog no. 4471252) was used for fragmented RNA, as reported previously (Frerich et al., 2017; Brown et al., 2017; Brayer et al., 2016). The Ion Proton S5/XL platform (Thermo Fisher Scientific) was used for sequencing at the Analytical and Translational Genomics Shared Resource Core at the University of New Mexico Comprehensive Cancer Center (UNM-CCC).

### Bioinformatics and Biostatistical Analyses

RNA sequencing (RNA-seq) analyses were aligned using tmap (v5.10.11) to a BED file that contained nonoverlapping exon regions from the UCSC genome browser (GRCh38/hg38). HTSeq (v0.11.1) was used to quantify exon counts (Pauken et al., 2021; Anders et al., 2015). The gene-level counts were generated by averaging counts across exons. Normalization of the library size and differential analysis were carried using edgeR (Pauken et al., 2021; Alexa & Rahnenfuhrer, 2016). Heatmap and cluster analysis were conducted using Heatmap3. Pathway enrichment analyses were executed using clusterProfiler, Pathview, and topGO software programs (Pauken et al., 2021; Alexa & Rahnenfuhrer, 2016). Data generated by pathway discrimination analyses were analyzed by the Reactome pathway database, as described previously (Croft et al., 2011).

### Cell Culture

Highly brain-metastatic melanoma CTC-derived clonal cells (70W-SM3; generated in Dr. Marchetti's laboratory (Vishnoi et al., 2018)) or the human melanoma MeWo line (ATCC; catalog no. HTB-65) were stored in liquid nitrogen and freshly recovered prior to use. Cells were maintained at 37° C. in a humidified 5% CO₂ incubator in DMEM nutrient mixture F-12 (DMEM/F12; Gibco, catalog no. 11320033), supplemented with 10% FBS (Gibco, catalog no. A4766801). Cells were grown using ultra-low attachment plates (Corning, catalog no. CLS3471), routinely tested for Mycoplasma using Mycoplasma Detection Assay (MycoAlert, Lonza) every 20 passages, and were only used at low-passage number (lower than 30 passages). PCR-based assay for authentication of cell lines was performed routinely. Luciferase-tagged 70W-SM3 cells were acquired using procedures reported previously (Lee & Wu, 2011). Prior to use, cells were checked for phenotypic changes using microscopy.

### CTC/CTC Cluster Capturing

Peripheral blood (7.5 mL) was collected from patients in EDTA-coated tubes and loaded onto the CTC Parsortix microfluidic chip (8 μm) within 1 hour of blood draw. Samples were analyzed employing the CTC filtration and/or microfluidic Parsortix PR1 instrument (Angle Europe Ltd.), and 6.5 μmol/L cartridges (Angle PLC). Following cassette priming, blood went through the cassette capturing single CTCs and CTC clusters based upon their size and deformability. To analyze captured CTC/CTC clusters, cells were either harvested and subjected to RNA isolation, or immunostained inside the Parsortix separation cassette, according to manufacturer's instructions (Sprouse et al., 2019). CTCs were defined and enumerated based upon positivity for human Mel-A (Alexa Fluor 594-tagged, Santa Cruz Biotechnology, catalog no. sc-20032), and human DAPI (Thermo Fisher Scientific, catalog no. D3571) staining, however negative for human CD45 (FITC-tagged, BioLegend, catalog no.103108) staining. Parsortix-captured cells displaying the human Mel-A⁺/DAPI⁺/CD45⁻ phenotype with a round and intact morphology were designated as CTCs. Confocal microscopy was performed for CTC visualization and enumeration of CTC/CTC clusters using Zeiss LSM800 microscope (10-40× magnification) and ZEN system software (Carl Zeiss Microscopy).

### CDXS

All in vivo studies were performed according to the approved Institutional Animal Care and Use Committee protocol. Animal studies were carried out using 6 to 12 weeks old immunodeficient NOD.Cg-Prkdcscid Il2rgtm1Wjl/SzJ (NSG) mice (Jackson Labs). Mice were given 50 μL (4 mg/mL) low-molecular weight heparin intravenously (retro-orbital or tail vein) 10 minutes prior to intracardiac injection of MBM CTC-derived clone (70W-SM3-Luc2 cells) to prevent thromboembolism in mice (Stocking et al., 2009). For intracardiac injections, mice were anesthetized with isoflurane (2.5%, 1 L/minute O₂ flow), placed in dorsal recumbency, and injected into the left ventricle (5.0×10⁵ cells in 50 μL of PBS) using a sterile 0.5-mL U-100 insulin syringe with a 29G×½″ needle (Beckton Dickinson, catalog no. 58324702). The injection site was confirmed as intracardiac by blood backflow into the syringe prior to injection. Animals were then monitored on a daily basis for changes in health status (rapid weight loss, distress, difficulty with breathing or ambulation, impaired mobility, seizures, ruffled coat, difficulty in obtaining food or water, etc.). For CTC capture and enumeration in animals over time, blood (100-150 μL) was collected from mouse retro-orbital sinus using EDTA-coated glass Pasteur pipette into a Mini-Collect tube (Greiner Bio-One, catalog no. K3E K3EDTA). Prior to blood collection, mice were anesthetized with isoflurane (2.5%, 1 L/minute O₂ flow). Tumor development was monitored weekly by Xenogen IVIS Spectrum animal imager (PerkinElmer), with acquisition of both two-dimensional and three dimensional (3D) optical tomography using Living Image Software program (PerkinElmer). For in vivo assessment of tumor burden, luciferin (150 mg/kg) was administered intraperitoneally into a mouse 10 minutes prior to imaging. At the end of the study, mice were sacrificed, necropsied, and weighed, and blood (0.6-1.0 mL) was collected via retro-orbital injection into an EDTA-containing MiniCollect tube (Greiner Bio-One, catalog no. K3E K3EDTA). Mice were kept under isoflurane anesthesia (5%, 1 L/minute O₂ flow), until opening the chest cavity. Liver, lungs, and brain organs were snap-frozen in Tissue-Tek OCT compound (Sakura Finetek USA Inc., catalog no. 4583). Spleen, sternum, femur, and skull-cap tissues were fixed in 10% neutral buffered formalin for pathologic evaluation. Because most melanoma cells produce melanin, melanoma metastasis was visually detected as brown-to-black pigmented regions (Lin & Fisher, 2007).

### MRI

Animals whose MBM was detected 24-hour postinjection of CTC-derived clonal cells (70W-SM3) were selected for MRI. MRI was conducted biweekly using the advanced Bruker 7 Tesla PET/MRI instrument (Bruker Inc.) to detect and monitor melanoma progression in the brain. The first MRI session was 3 days postinjection and considered day 0 of MRI studies. MRI was used to assess the presence of tumors in Gadolinium contrast-enhanced (CE) T1-weighted (T1W) and brain structures in T2-weighted (T2W) MRI. Image resolution for T1W and T2W MRI was 100×100×500 μm³. The skull stripping technique was performed on the T2WMRI sequence to remove extrameningeal tissues from brain images of the whole head and to better visualize tumors. T2-weighted images were acquired with a fast spin-echo sequence rapid acquisition with relaxation enhancement with repetition time (TR)/echo time (TE)=5,000 ms/30 ms, field of view (FOV)=15 mm×15 mm, slice thickness=0.5 mm, interslice distance=0.5 mm, number of slices=30, matrix=150×150, number of average=1. T1-weighted images were acquired with a 3D fast low angle shot with TR/TE=20 ms/5 ms, FOV=15 mm×15 mm×15 mm, slice thickness=0.5 mm, interslice distance=0.5 mm, number of slices=30, matrix=150×150, number of average=9. Fast T1 maps were developed using inversion recovery (IR) based T1_EPI (echo planar imaging) with RT/TE=3,000 ms/10.2 ms, FOV=15 mm×15 mm×15 mm, slice thickness=0.5 mm, interslice distance=0.5 mm, number of slices=30, matrix=100×100, number of average=1, EPI segments=8, automatic ghost correction=on, IR offset=20, IR Spacing=160, IR points=16 (Ordidge et al., 1990; Freeman et al., 1998).

Prior to MRI, mice were given 100 μL (3.89 mL/kg) of contrast agent Multi-Hance gadobenate dimeglumine (Bracco Diagnostics Inc, catalog no. SP9002A) intravenously (retro-orbital or tail vein) to enhance tumor visualization. Contrast agent was injected right before placing the animal into the MRI scanner. The mouse was positioned in a dedicated holder and placed in the isocenter of the 7T MRI scanner (Bruker Biospin MRI), which was equipped with a 30 cm bore, a 20 cm gradient with the strength of 660 mT/m and shim systems (Bruker Biospin MRI). To obtain a good signal-to-noise ratio, a small-bore linear RF coil (inner diameter=72 mm), and a phased-array surface coil were employed for signal excitation and detection, respectively. During MRI experiments, mice were anaesthetized with 1-1.5% isoflurane (Phonenix, Clipper Distributing Company) by mechanical ventilation. A monitoring system of physiologic parameter (SA Instruments, Inc) enabled the visualization of the respiratory cycle.

### MRI Analyses and Statistical Validation

MRI analyses were performed by the Radiology Department at UNM-HSC by one of the co-authors (E. Taylor). Images were organized by scan date and subject number, followed by whole brain bias field correction using the Advanced Normalization Tools software in Python (ANTsPy; Python Software Foundation; (Fedorov et al., 2012)). CE-T1W MRI was analyzed by 3D Slicer software (Linux, version 4.11.20210226). Brain tumors were semi-manually segmented using the level tracing method for tumor volume measurement (Fedorov et al., 2012). T2W MRI was skull-striped (SS) by a deep learning technique with U-Net followed by manual correction of the SS image in 3D slicer. Brain atlas with 62 regions structures including frontal lobe (FL), parieto-temporal lobe (PTL), and other major brain regions (Dorr et al., 2008) was spatially normalized to T2Wimages inANTsPy by rigid, affine, and a deformable registration for each individual subject and time point was carried out. Total brain tumor volume and regional brain tumor volume were then calculated from segmented CE-T1W MRI labeled with the brain atlas. Brain tumors were counted using scikit-image (Van der Walt et al., 2014) measure label tool to assign all 3D connected regions with a unique integer value in Python. Brain atlas labels were then referenced to assign each tumor>10 voxels to a brain region of interest.

### Data Availability

NCBI SRA database BioProject accession number PRJNA866169.

### Results

Patient CTCs exhibit extensive heterogeneity in their cell surface biomarkers (Vishnoi et al., 2018; Alexa & Rahnenfuhrer, 2016; Khoja et al., 2014). The absence of a universal CTC biomarker is particularly valid in melanoma (Vishnoi et al., 2018), creating a challenge for the detection and capture of the entire spectrum of CTC subsets present and implicated in melanoma carcinogenesis and metastasis (Vishnoi et al., 2018; Khoja et al., 2014; Joosse et al., 2015). Multiple CTC platforms have been used to detect and isolate melanoma CTCs, including CellSearch (Luo et al., 2014; De Giorgi et al., Hong et al., 2018). CellSearch is the only FDA-cleared platform for CTC isolation, visualization, and interrogation [FDA clearance is however applicable only for metastatic breast, prostate, and colorectal cancers, not melanoma (Alex-Panabieres & Pantel, 2014; Vishnoi et al., 2018; Joosse et al., 2015)]. Specifically, the melanoma CellSearch CTC kit uses MEL-PE (CD146) biomarker to capture CTCs. Captured CTCs are then detected, visualized, and enumerated via automated CellBrowser software. Accordingly, a consequence of melanoma CTC heterogeneity is inability of the CellSearch assay to isolate and study the entire CTC spectrum beyond MEL-PE⁺/DAPI⁺/CD45⁻ cells.

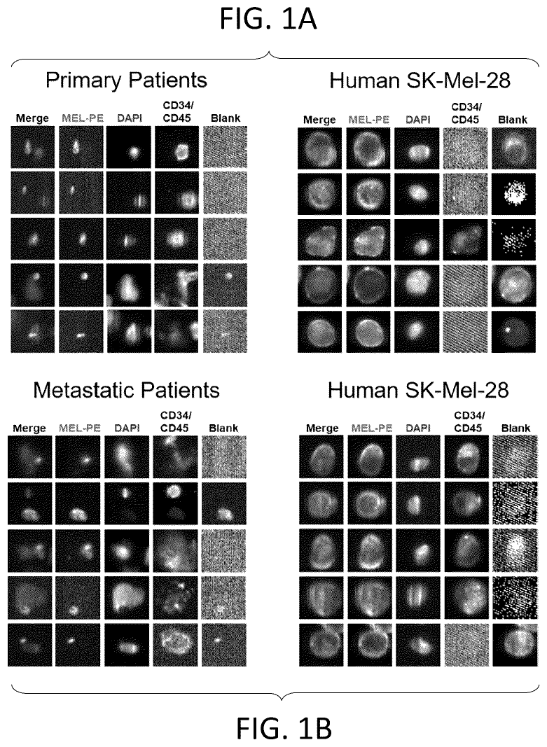

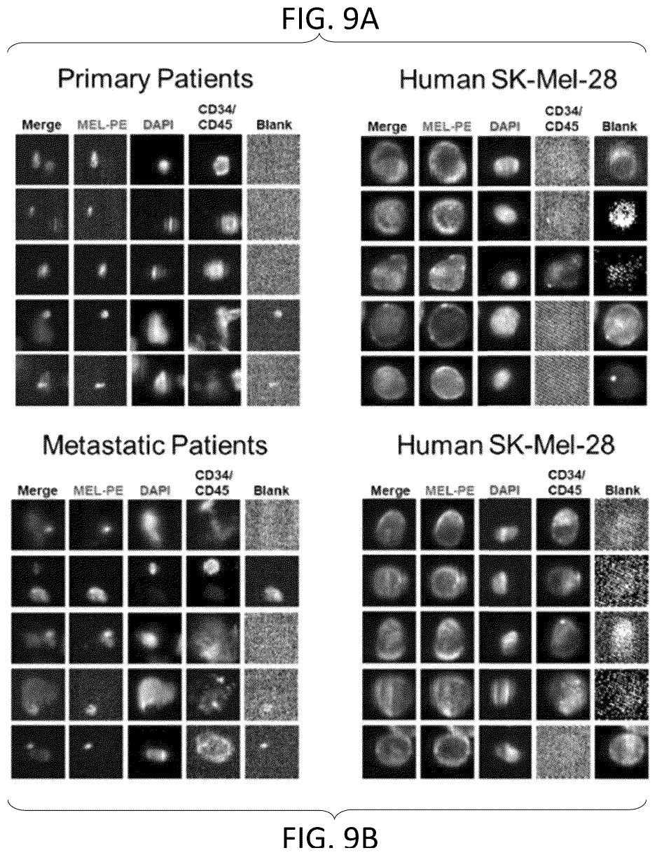

As first step, peripheral blood from patients with primary or metastatic melanoma was collected and evaluated by CellSearch. No CTCs could be detected by the CellSearch platform in any of these analyses (FIGS. 1A and B). To confirm validity of these results, human melanoma cells (SK-Mel-28 line) embedded within the CellSearch melanoma CTC assay and run in parallel to patient samples showed a high number of CTCs being captured (positive control; FIG. 1). Healthy donors' blood was analyzed via CellSearch with negligible results (negative control; FIG. 9A). Similar CellSearch analyses using the CTC-derived melanoma clone (70W-SM3cells) spiked in healthy donors' blood at different concentrations displayed consistent CTC capturing and/or visualization (FIG. 9B). These findings suggest that CellSearch cannot detect melanoma CTCs in patient samples based solely on the presence of the MEL-PE (CD146) biomarker selection.

Consequently, a multilevel approach was selected to characterize CTCs and evaluate a CTC-associated gene signature responsible for MBM onset. To discriminate gene expression differences among CTC populations in patients with primary and metastatic melanoma, multiparametric flow cytometry (FACS) was implemented to deplete circulatory normal cell lineages (Lin+ or LinP cells) from peripheral blood of patients, thus selecting a cell population of neoplastic origin (referred as Lin− or LinN cells here and onward; Vishnoi et al., 2018).

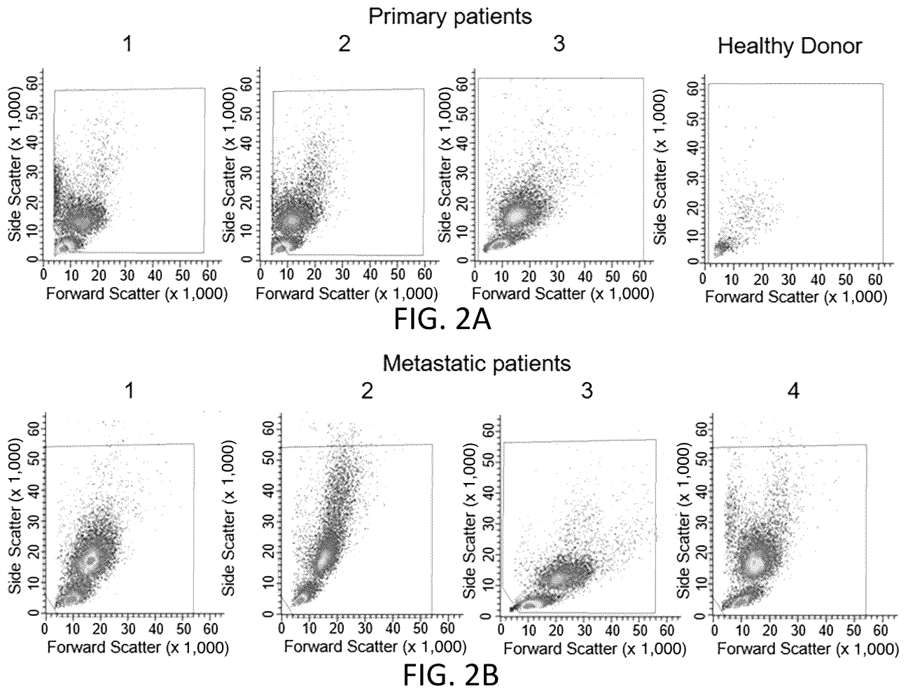

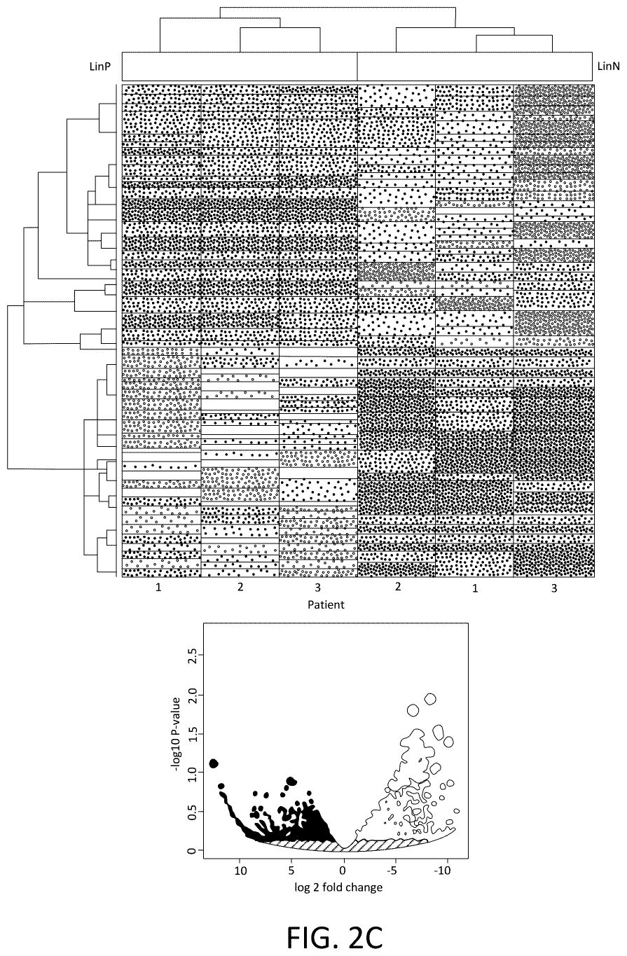

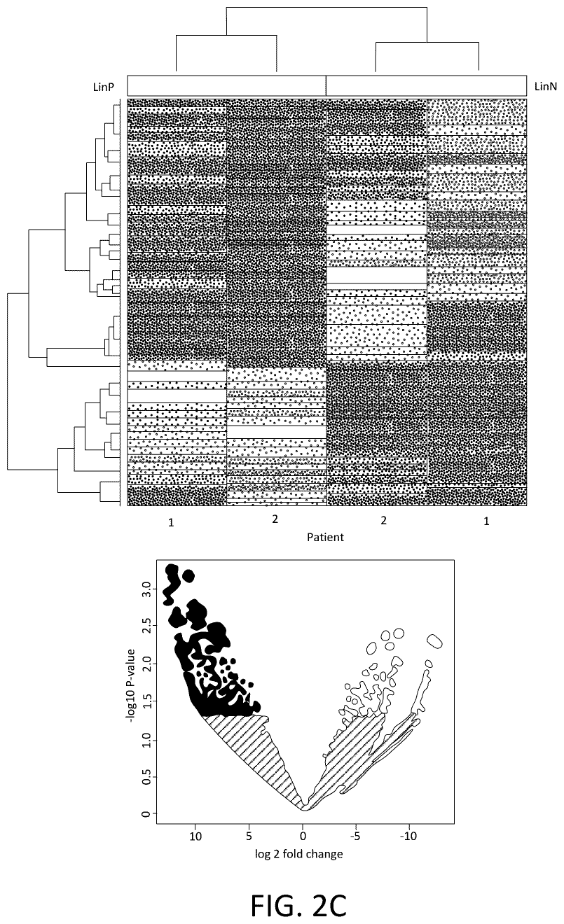

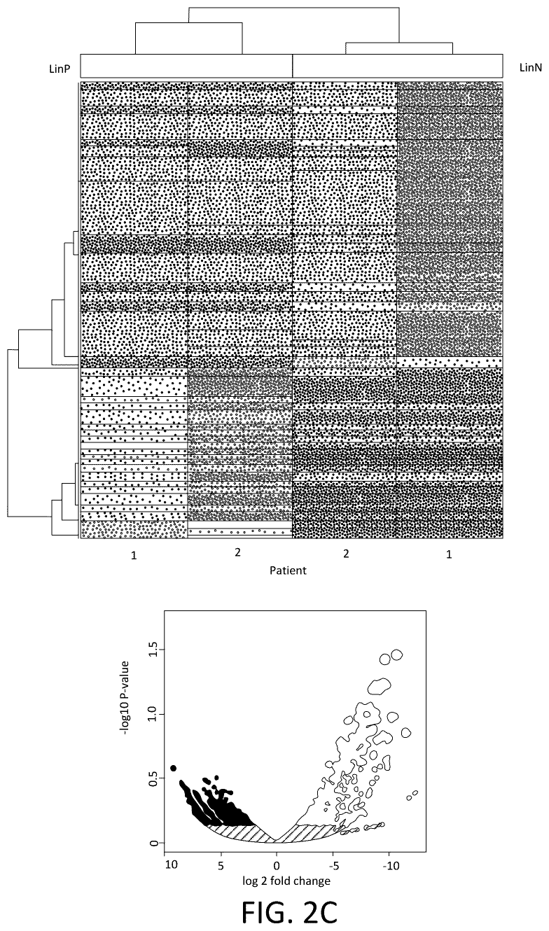

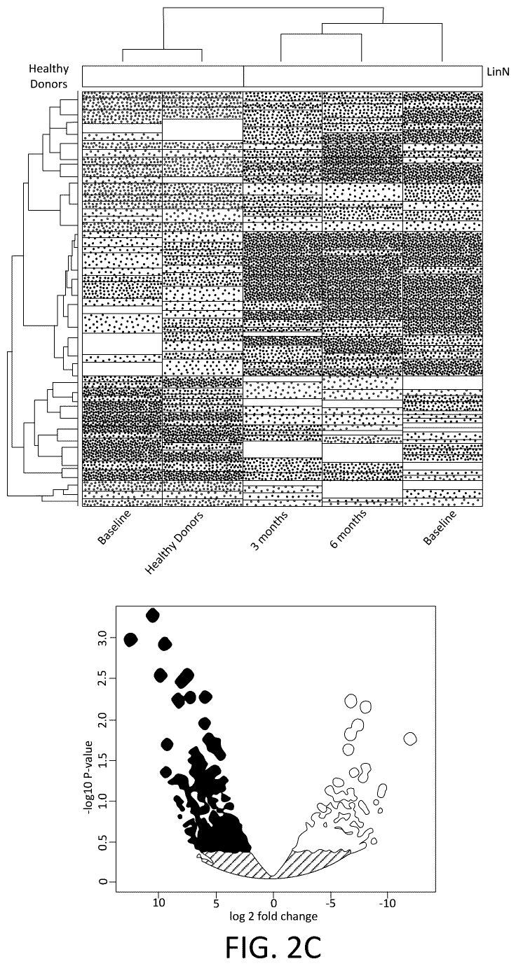

Next, RNA-seq was performed on FACS-sorted Lin−/Lin+ cells to assess whether Lin− cell populations isolated from primary melanoma without clinical evidence of metastasis or Lin− cells isolated from patients with metastatic melanoma regardless of MBM could reflect the evolution of melanoma in the blood (FIG. 2). Normal blood served as negative control (FIG. 2A). The negative depletion strategy was carried to isolate CTC-enriched Lin-fraction from the Lin+ cell population for every sample. Analyses of Lin−/Lin+ samples from patients with and/or without MBM were performed in parallel to compare Lin− gene signatures from patients (FIGS. 2A and B). Not all metastatic patients exhibited brain metastasis. The metastatic sites for each patient are presented in Table 1. Specifically, patients with MBM had brain metastasis, while patients with No MBM had metastasis to distant organs, but not to the brain. Hierarchical clustering not only showed the distinction among Lin−/Lin+ cell transcriptomes, but also significant differences among Lin− cell fractions at distinct stages of melanoma progression to MBM, reflecting CTC/Lin− heterogeneity (FIG. 2C). Of note, an extensive (0-6 months) longitudinal investigation of Lin− transcriptomics was performed in a patient with MBM to evaluate gene expression signatures relatable to MBM progression within the same individual (FIG. 2C). This patient with known MBM underwent treatment (nivolumab) and periodic MRIs which did not show any new or progressive intracranial metastatic lesions.

RNA-seq analyses of these samples were performed, and unsupervised hierarchical clustering revealed distinct transcriptomic profiling of the CTC-enriched Lin− fraction in all four analyses (FIG. 2C). Furthermore, detailed transcriptomic analyses of the Lin− fraction of patients with MBM and the longitudinal monitoring of an individual patient with MBM were integrated with MBM mouse transcriptomics data to yield common upregulated and/or downregulated genes, and to identify common gene signatures using a four-level discrimination approach discussed below.

### Spatial and Temporal Divergence of CTC-MBM Transcriptomic Signatures

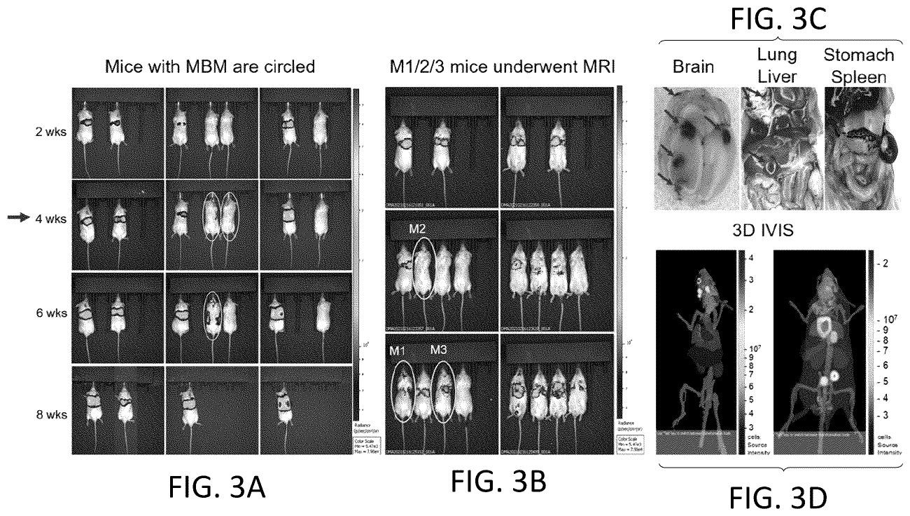

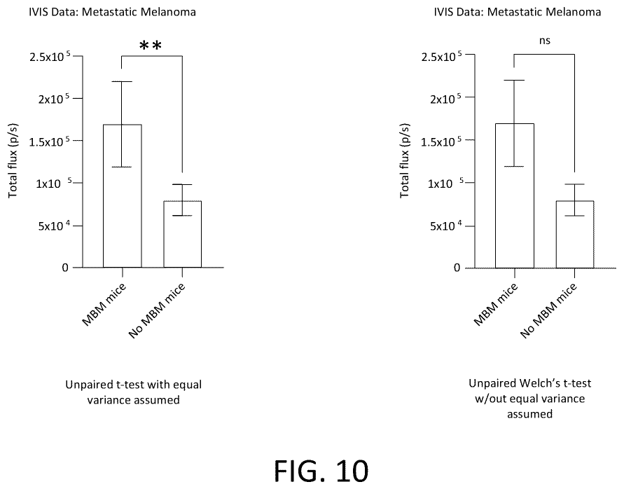

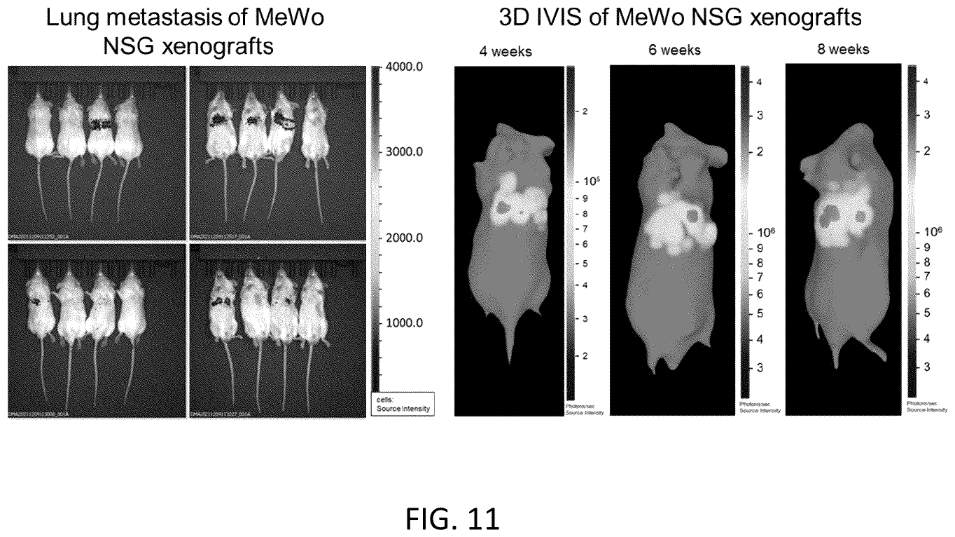

As next step, MRI was employed to develop the first CTC-driven, MRI associated CTC xenograft model (MRI-MBM CDX; FIG. 3). While MBM was consistently identified at 4 weeks following 70W-SM3 cell injection; in one group employing 10 male NSG mice, 3 presented MBM IVIS as early as 24 hours (FIGS. 3A and B). Total flux of MBM signal in animal brains was quantified by IVIS and confirmed to be higher in these mice compared with ones without MBM (FIG. 10). Accordingly, these animals were selected for sequential MRI, while the remaining 7 mice underwent weekly IVIS imaging parallel to MRI to monitor MBM occurrence and progression. Two mice developed MBM at 4-week point post-injection while another mouse presented with MBM at 8 weeks (FIG. 3A). 3D IVIS virtual tomography was performed to reconstruct brain tumors in 3D with the identification of multiple MBM (FIG. 3D). Mouse necropsies confirmed multiple brain metastatic sites, along with metastatic spread to lungs, liver, stomach, and spleen (FIG. 3C). Because of the high metastatic burden, mice were sacrificed at 8-10 weeks postinjection. However, MBM-IVIS signal specificity for the CTC-derived clone (70W-SM3 cells) was confirmed by parallel analyses employing human melanoma cells (MeWo) which are known to metastasize to lung but not to brain (Thies et al., 2007). Lung metastasis but no MBM was detected in MeWo-injected animals (FIG. 11).

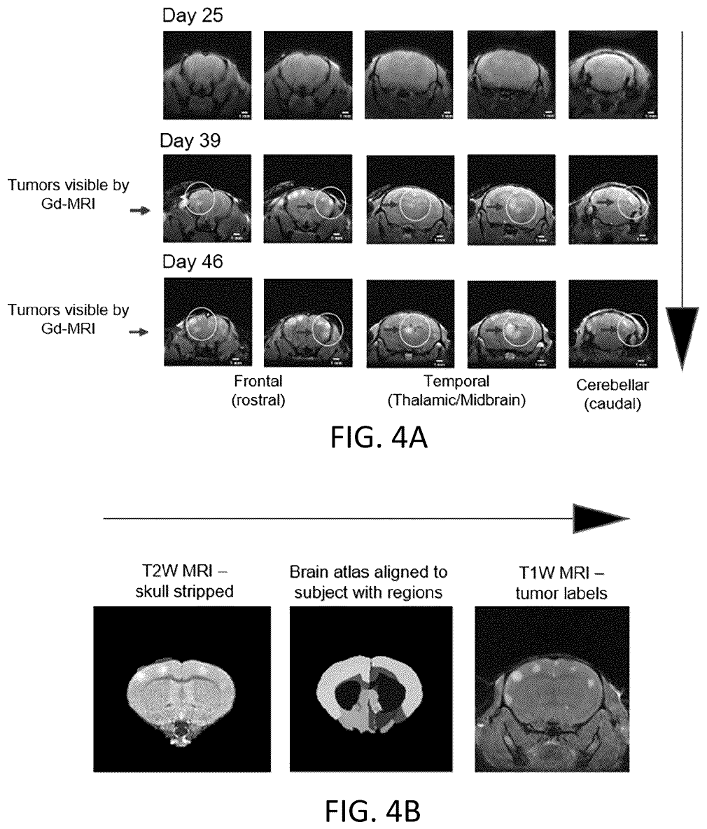

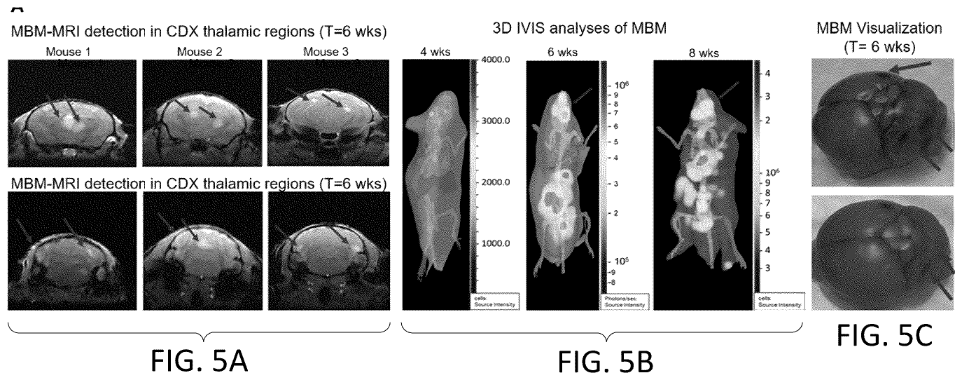

Longitudinal MRI (FIG. 4) was performed biweekly to monitor MBM progression and to determine any ensuing MBM. MRI was carried out using the advanced 7-Tesla MRI scanner with high signal-to-noise ratio, translating into enhanced resolution and improved differentiation among brain tissue (Platt et al., 2021). No brain masses were visible by MRI by the third timepoint (25 days postinjection; FIG. 4A); however, MBM was MRI detectable at day 39 postinjection in all 3 animals (FIGS. 4B and 5). Importantly, tumors localized to specific regions of the brain—FL, PTL, and cerebellum—which recapitulated MBM clinical presentation (FIG. 5A), validating the MRI-MBM CDX model for CTC MBM regional specificity (FIG. 5C). Longitudinal 3D IVIS tomography was executed to reconstruct brain tumor development in 3D over the period of 8 weeks (FIG. 5B).

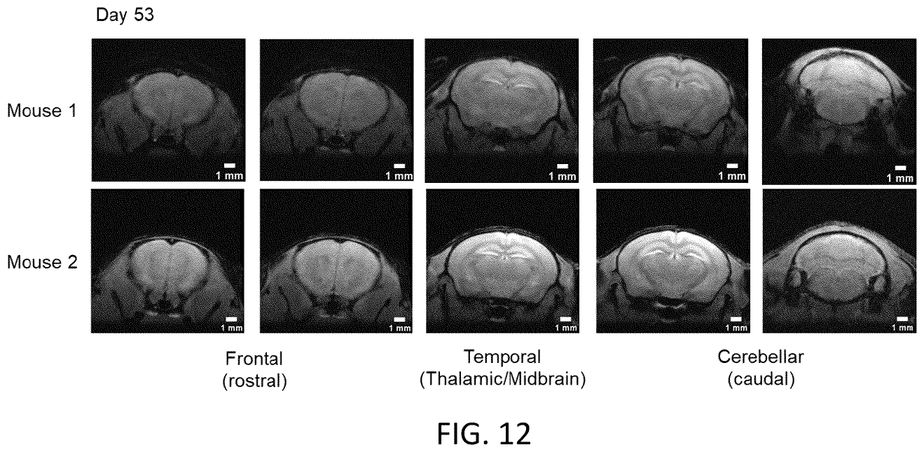

Furthermore, MRI-detectable tumor volume was quantified for each region and animal, with FL having the highest tumor burden (Table 2). Sequential MRI at day 46 postinjection showed a significant increase of tumor mass in all MBM sites (FIG. 4A; Table 2A). Moreover, the average value in tumor volume was calculated by brain region from day 39 to day 46 postinjection (Table 2B). The highest values in brain tumor volume were observed in FL, followed by cerebellum and PTL. It was complemented by employing the brain atlas with 62 brain regions normalized to T2W images using ANTs Python, and segmented CE-T1W MRI was implemented to quantify brain tumor volume (Table 2B). Negative controls consisted of performing MRI of mice without IVIS-detectable MBM, confirming no MRI-MBM detection (FIG. 12).

### Tables 2A and 2B

A. Spatial/temporal progression of MBM

| Mouse | 1 | 2 | 3 | 1 | 2 | 3 |
|---|---|---|---|---|---|---|
| Days | 39 | 39 | 39 | 46 | 46 | 46 |
| Cerebellum | 0.65 | 1.15 | 0.53 | 1.61 | 1.84 | 2.44 |
| (mm³) | | | | | | |
| FL (mm³) | 4.06 | 1.85 | 0.56 | 4.76 | 8.08 | 2.37 |
| PTL (mm³) | 1.00 | 0.93 | 0.98 | 1.58 | 1.26 | 1.86 |

B. Brain tumor volume change (ratio)

| Brain region | Mean | SEM |
|---|---|---|
| Cerebellum | 2.91 | 0.91 |
| FL | 3.44 | 1.15 |
| PTL | 1.62 | 0.16 |

Spatial and temporal growth of MBM. Table A shows analyses of spatial and temporal MRI-MBM progression over time in various brain regions (FL=Frontal Lobe; PTL=Parietotemporal Lobe). MBM volume/ratios and statistical validation (SEM) are presented in Table B.

### Longitudinal CDX CTC Levels are MBM Dependent

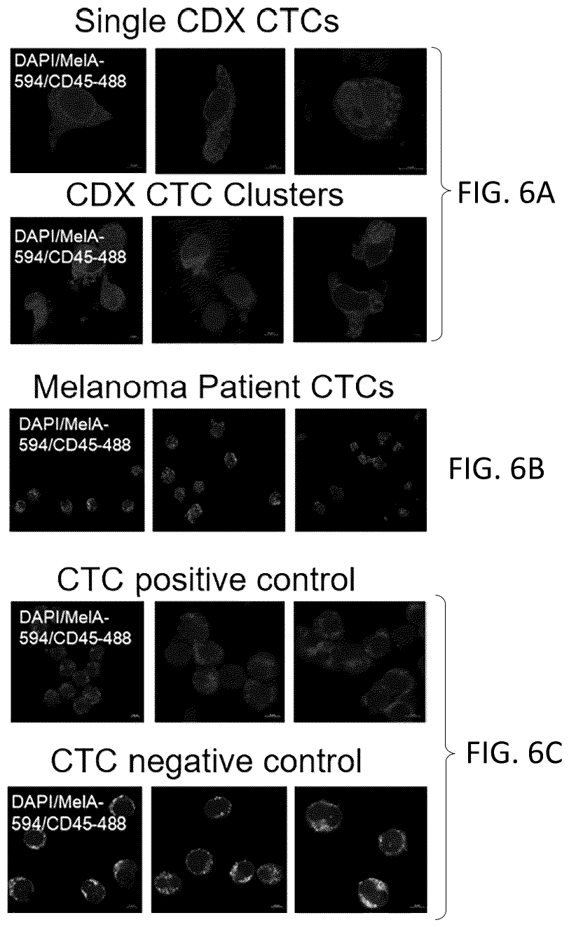

To determine the correlation between MRI-MBM and CTC content in the CDX model, CTCs from MBM/No MBM mice were captured and interrogated longitudinally by retro-orbital blood (150 μL) collection. Blood from three MRI-MBM CDXs was combined following each blood draw and analyzed by the CTC Parsortix microfluidic device to capture single CTCs and CTC clusters based upon their size and deformability. Parsortix-captured CTCs were immunostained for human Mel-A Alexa Fluor 594, human FITC-CD45, and DAPI (markers have been used to define human melanoma CTCs as Mel-A⁺/DAPI⁺/CD45⁻ cells; Bretones et al., 2018; Sprouse et al., 2019) within the Parsortix separation cassette, visualized and counted (FIG. 6A). Interestingly, while CTCs were not detected in murine blood for the first 4 weeks (Table 3), CTCs could be captured at 6 weeks, and this correlated with the MRI-MBM detection in these animals (FIG. 6A). Second, considerable increase of CTC numbers was observed at 8 weeks postinjection, when the number of single CTCs increased 4-fold. Third, homotypic CTC clusters were also detected at this time, either small (2, 3, 4 cells) or large (5 cells or greater) which are pivotal since they have stronger metastatic potential and higher resistance against therapy than single CTCs (Amintas et al., 2020; Au et al., 2016; FIG. 6; Table 3B). These findings were also consistent with the increase of brain tumor burden in these animals at the last MRI timepoint, suggesting that growing MBM promoted shedding of higher CTC numbers into the bloodstream of MRI-MBM CDXs, and confirmed the severity of MRI temporal and spatial detection (FIG. 4). These results were complemented by multiple Parsortix CTC analyses involving: (i) No MBM CDXs (but with metastasis to other organs) which showed detection of CTCs at 6 weeks; however, no significant increase in CTC number or presence of CTC clusters were observed in 2 weeks (Table 3C); (ii) metastatic but with no MBM patient blood samples which correlated with the above findings, for example, a patient possessing high number of single CTCs (77 CTCs per 100 μL of blood; FIG. 6B; Table 3A); (iii) blood from healthy donors spiked with CTC-derived clonal cells at increasing concentrations which were correlative with increasing numbers of CTCs; (iv) healthy donors' blood resulting in no CTC detection (FIG. 6C).

### Tables 3A-3C

A. Parsortix quantification of patient samples

| Clusters (CTCs | Metastatic Patients | | | Primary Patients | | |
|---|---|---|---|---|---|---|
| per 100 uL) | 1 | 2 | 3 | 1 | 2 | 3 |
| Single cells | 67 | 77 | 52 | 4 | 3 | 3 |
| 2-cell | 6 | 4 | 1 | 0 | 1 | 0 |
| 3-cell | 2 | 2 | 0 | 0 | 0 | 0 |
| 4-cell | 0 | 0 | 0 | 0 | 0 | 0 |
| 5-cell or greater | 0 | 0 | 0 | 0 | 0 | 0 |

B. CDX with MBM

| Clusters | | | | | |
|---|---|---|---|---|---|
| (CTCs | | | | | |
| per 100 | | | | | |
| uL) | 3 days | 2 wks | 4 wks | 6 wks | 8 wks |
| Single | 0 | 0 | 0 | 4 | 16 |
| cells | | | | | |
| 2-cell | 0 | 0 | 0 | 0 | 8 |
| 3-cell | 0 | 0 | 0 | 0 | 4 |
| 4-cell | 0 | 0 | 0 | 0 | 4 |
| 5-cel or | 0 | 0 | 0 | 0 | 4 |
| greater | | | | | |

C. CDX with no MBM

| Clusters | | | | | |
|---|---|---|---|---|---|
| (CTCs | | | | | |
| per 100 | | | | | |
| uL) | 3 days | 2 wks | 4 wks | 6 wks | 8 wks |
| Single | 0 | 0 | 0 | 4 | 4 |
| cells | | | | | |
| 2-cell | 0 | 0 | 0 | 0 | 0 |
| 3-cel | 0 | 0 | 0 | 0 | 0 |
| 4-cell | 0 | 0 | 0 | 0 | 0 |
| 5-cell or | 0 | 0 | 0 | 0 | 0 |
| greater | | | | | |

Enumeration of CTCs captured by Parsortix. A, Quantitation of CTCs from metastatic melanoma patients not diagnosed with MBM (No MBM). Higher CTC numbers were captured and visualized by the CTC Parsortix platform in MBM (B) vs No MBM CDXs (C) over time and consistent with MRI-MBM/pathological detection.

### The Multilevel CTC Transcriptomic Characterization of MBM CDXs

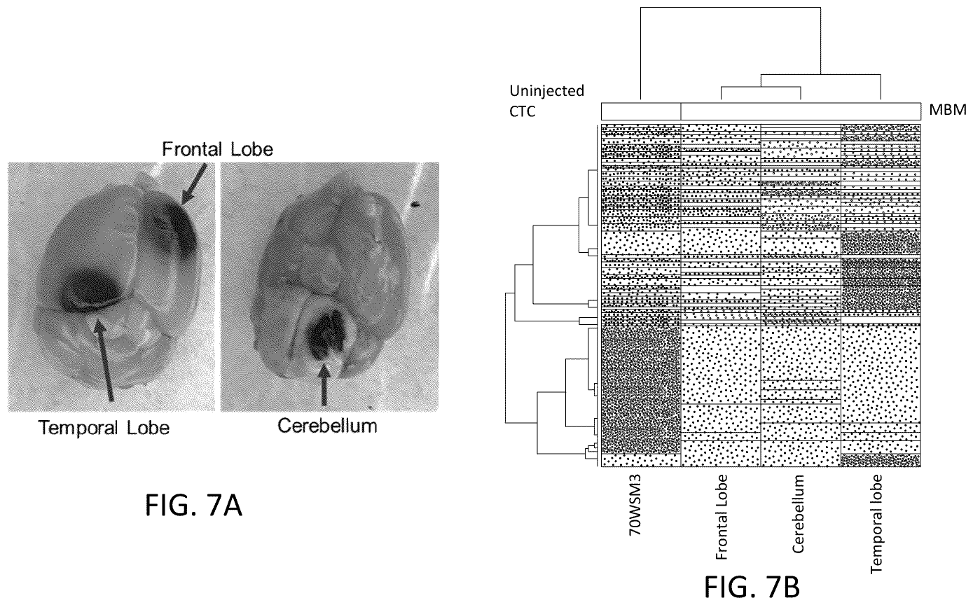

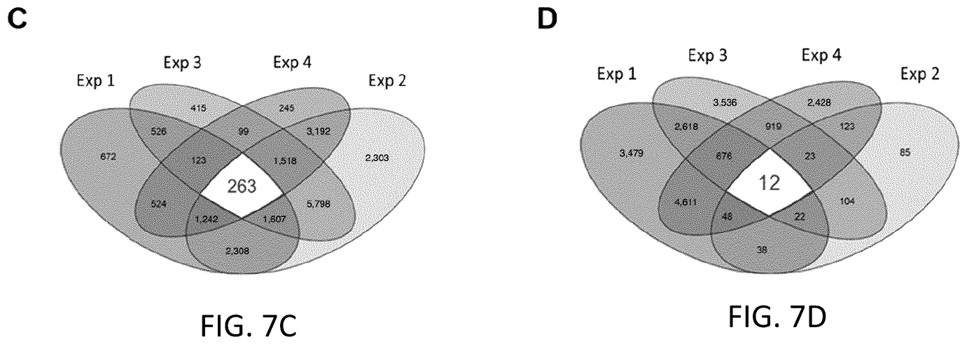

Analyses of gene expression patterns in patients with MBM and patients without MBM indicated distinct differences in their clustering patterns (FIG. 2C). These differences prompted the investigation of the variability of gene expression levels in blood of CDX mice with MBM versus animals without MBM. MRI-MBM CDXs mice could not be used in these analyses because CTCs were captured and/or immunostained within the Parsortix cassette, and therefore not accessible to further investigations. Experiments were conducted involving the injection of NSG mice with the highly brain-metastatic CTC-derived clone (5×10⁵ cells/mouse, 6 mice/subgroup), monitoring metastatic development by weekly IVIS imaging. Augmented tumor burden was detected over a period of 8 weeks (2 mice developed MBM), afterward necropsies of MBM mice were performed to identify specific MBM sites and blood was collected. Blood samples were then analyzed by Parsortix (no immunostaining) to harvest CTCs for RNA-seq interrogation. Conversely, the remaining 4 mice developed metastasis to other organs, for example, liver, spleen, etc., but not to brain, and were similarly processed. Consistent with MRI findings, MBM CDXs developed tumors in FL, PTL, and cerebellum (FIG. 7A). Single-cell RNA-seq was executed to compare gene expression levels in different regions of the brain, with libraries aligned to the human and not mouse genome. Resulting heatmaps displayed significant variation among brain regions, with distinct patterns which were significantly different from uninjected CTC-derived clonal cells (FIG. 7B). Results suggest that changes in molecular pathways occur upon the successful CTC MBM onset are region specific.

### The Identification of the CTC RPL/RPS Gene Signature by Multilevel MBM Discrimination

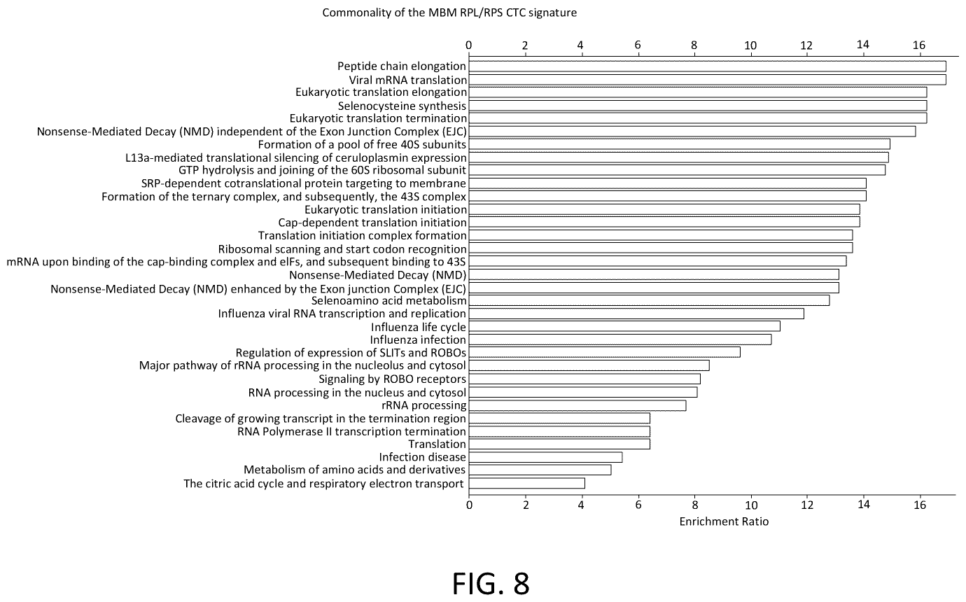

To identify a unique CTC genetic signature associated with MBM, bioinformatics analyses involving unsupervised transcriptomic profiling of MBM detected in patients and animal samples were performed, employing a four-pronged approach to identify a common CTC MBM signature. Specifically, this consisted in CTC gene expression analyses involving: (i) primary, metastatic (No MBM), and patients with MBM, (ii) CTC longitudinal profiling (9 months period) in a patient diagnosed with MBM; (iii) blood from MBM/No MBM CDXs; and (iv) MBM CDX tissues spatially distinct (FL, PTL, and cerebellum). Transcriptomes were mapped and/or analyzed altogether to yield 263 common upregulated and 12 downregulated genes of MBM (FIGS. 7B and D, respectively). Furthermore, reactome analyses against the hallmark gene sets generated a list of statistically significant pathways involved in MBM onset and progression (FIG. 8). Notably, 26 of 33 gene pathways had 21 commonly shared genes (Table 4), with all these genes being members of the large or small ribosomal proteins (RPL/RPS) gene families and involved in translational processes: the CTC RPL/RPS gene signature of MBM (FIG. 8—highlighted in yellow). Of note, nine RPS common genes were shared among higher number of pathways and were found in 30 of 33 pathways. Furthermore, RPL/RPS genes were highly significant in multilevel analyses: the top 20 genes out of 263 total upregulated genes included nine RPL/RPS-related genes (Table 5). Equally relevant, patients with primary melanoma and metastatic patients with No MBM (FIG. 2) did not possess high RPL/RPS gene expression markers, in striking contrast from patients diagnosed with MBM: mean RPL/RPS values in patients with MBM had 2- to 10-fold increase in the level of ribosomal proteins, compared with patients with No MBM (Table 6). Significantly elevated RPL/RPS expression was also detected in most molecular pathways involved in translational programs known of fundamental importance in cancer progression (FIG. 8; Elhamamsy et al., 2022; Cao et al., 2022).

TABLE 4
The RPL/RPS CTC Signature

| | |
|---|---|
| RPL 12 | ribosomal protein L12 |
| RPL 13 | ribosomal protein L13 |
| RPL 18A | ribosomal protein L18A |
| RPL 19 | ribosomal protein L19 |
| RPL 23 | ribosomal protein L23 |
| RPL 26 | ribosomal protein L26 |
| RPL 35A | ribosomal protein L35A |
| RPL 37 | ribosomal protein L37 |
| RPL 38 | ribosomal protein L38 |
| RPL 6 | ribosomal protein LG |
| RPL 7 | ribosomal protein L7 |
| RPL 7A | ribosomal protein L7A |
| RPS 12 | ribosomal protein S12 |
| RPS 15A | ribosomal protein S15A |
| RPS 18 | ribosomal protein S18 |
| RPS 24 | ribosomal protein S24 |
| RPS 26 | ribosomal protein S26 |
| RPS 28 | ribosomal protein S28 |
| RPS 5 | ribosomal protein S5 |
| RPS 7 | ribosomal protein S7 |
| RPS A | ribosomal protein SA |

The CTC RPL/RPS gene signature of MBM. Table 4 shows the RPL/RPS CTC gene signature as result of the four-pronged hierarchical clustering among all samples and translational pathways analyzed (Reactome pathway database). The 21 RPS/RPL genes of the commonly-shared CTC gene signature of MBM are listed.

TABLE 5
Top 20 upregulated genes in MBM

| Gene Name | EXP1 | EXP2 | EXP3 | EXP4 |
|---|---|---|---|---|
| BIRC7 | 2.300318 | 2.330309 | 2.307413 | 3.946502 |
| CDM3 | 3.939986 | 4.920184 | 4.767726 | 2.87185 |
| CLK1 | 3.262489 | 1.78731 | 1.686732 | 1.736786 |
| CSPG4 | 3.757032 | 1.710669 | 3.083843 | 1.750102 |
| EIF4B | 3.773532 | 2.083414 | 3.294649 | 2.811043 |
| MRFAP1 | 3.398459 | 2.010767 | 3.889015 | 6.006767 |
| PAIP1 | 2.791706 | 1.953115 | 1.694996 | 2.052303 |
| PPDPF | 1.710868 | 2.810075 | 2.452767 | 2.18403 |
| RIMKLB | 2.245525 | 2.084877 | 4.393563 | 1.904588 |
| RPL12 | 3.840793 | 1.804809 | 1.689898 | 3.612129 |
| RPL13 | 3.315848 | 2.536863 | 1.817364 | 4.886132 |
| RPL18A | 3.482471 | 2.050779 | 1.572679 | 5.612618 |
| RPL19 | 1.892976 | 2.400079 | 2.2411 | 4.180579 |
| RPL7 | 2.761046 | 2.939965 | 4.56366 | 4.028576 |
| RPS12 | 2.031537 | 2.022204 | 1.649564 | 4.92244 |
| RPS18 | 2.383768 | 2.700824 | 2.410597 | 4.847084 |
| RPS24 | 3.729289 | 2.136777 | 3.129485 | 2.661536 |
| RPS26 | 2.324018 | 3.043821 | 4.441823 | 3.267062 |
| SPCS2 | 2.324018 | 2.257102 | 7.362976 | 2.22085 |
| SPRY4 | 2.644182 | 1.83693 | 4.738747 | 2.974235 |

Top 20 upregulated genes in MBM by the four-pronged experimental approach used in this study. Nine out of 20 upregulated genes are RPL/RPS genes of the MBM CTC signature.

TABLE 6
Individual and mean values of the CTC RPL/RPS signature by patient

| | Primary | Primary | Primary | MBM | MBM | No MBM | No MBM |
|---|---|---|---|---|---|---|---|
| Gene Name | patient 1 | patient 2 | patient 3 | patient 1 | patient 2 | patient 1 | patient 2 |
| RPL12 | 8.770696 | 51.224504 | 1.650823 | 6.868503 | 0.000000 | 0.000000 | 3.075507 |
| RPL13 | 49.954831 | 219.590315 | 20.299009 | 63.778952 | 112.459841 | 54.063239 | 19.551437 |
| RPL18A | 10.677368 | 1.191268 | 3.790779 | 18.643078 | 0.000000 | 0.000000 | 0.659037 |
| RPL19 | 2.288008 | 6.353427 | 3.729637 | 92.234177 | 24.822217 | 9.393227 | 11.862670 |
| RPL23 | 8.008026 | 9.530140 | 4.952469 | 62.797737 | 0.506576 | 6.063413 | 28.118921 |
| RPL26 | 0.000000 | 0.000000 | 0.000000 | 0.981215 | 14.184124 | 0.208738 | 0.219679 |
| RPL35A | 10.296034 | 14.295210 | 7.459276 | 31.398869 | 0.000000 | 2.296122 | 5.491977 |
| RPL37 | 67.114887 | 175.910506 | 6.114159 | 157.975558 | 341.938706 | 34.233093 | 13.620102 |
| RPL38 | 0.000000 | 0.397089 | 0.000000 | 10.793361 | 0.000000 | 4.174767 | 1.098395 |
| RPL6 | 6.864023 | 12.706854 | 0.366850 | 49.060732 | 0.000000 | 1.043692 | 1.977112 |
| RPL7 | 0.000000 | 13.601032 | 0.000000 | 64.948020 | 0.000000 | 0.208738 | 2.196791 |
| RPL7A | 6.482688 | 15.883567 | 26.780018 | 6.868503 | 287.228513 | 28.178680 | 9.226521 |
| RPS12 | 1.906673 | 3.176713 | 3.851920 | 0.000000 | 17.730155 | 2.922337 | 3.075507 |
| RPS15A | 18.685395 | 0.000000 | 13.145443 | 48.079518 | 0.506576 | 10.228180 | 5.272298 |
| RPS18 | 2.288008 | 15.883567 | 0.305708 | 65.741381 | 0.000000 | 0.626215 | 3.295186 |
| RPS24 | 12.584041 | 3.673803 | 0.489133 | 114.802114 | 0.000000 | 1.878645 | 4.832940 |
| RPS26 | 2.669342 | 0.000000 | 0.000000 | 7.849717 | 0.000000 | 0.000000 | 0.000000 |
| RPS28 | 0.000000 | 8.338873 | 0.000000 | 36.323727 | 2.532879 | 0.000000 | 3.295186 |
| RPS5 | 8.389361 | 3.573803 | 1.283973 | 20.605508 | 0.000000 | 0.626215 | 1.098395 |
| RPS7 | 2.669342 | 39.311829 | 0.000000 | 32.380083 | 0.000000 | 0.000000 | 0.000000 |
| RPSA | 28.218759 | 17.074835 | 5.688168 | 21.586722 | 0.506576 | 10.228180 | 3.295186 |
| MEAN | 11.803213 | 29.217404 | 4.968906 | 44.671088 | 42.205768 | 8.217700 | 6.208824 |

Individual and mean values (cpm) of RPL/RPS CTC MBM signature per patient analyzed. MBM patients showed higher mean values of RPL/RPS genes vs patients with No MBM.

### Discussion

This study centered on investigating the biology of CTCs associated with the onset and progression of MBM and provides first-time evidence of a specific CTC gene signature (“The CTC RPL/RPS gene signature”) associated with MBM. This was achieved by multilevel analyses, employing a novel MRI dependent MBM CDX model, the gene expression interrogation of CTCs/Lin− cell populations isolated from patients at distinct stages of disease progression (primary, metastatic melanoma diagnosed with or without MBM), CTC longitudinal monitoring (patient diagnosed with MBM), or by the interrogation of CDX MBM evaluated spatially or temporally. The multilevel approach included comparing blood samples of metastatic patients with brain metastasis (MBM) versus metastatic patients with tumor cell dissemination to non-brain distant sites, for example, lungs, but not to brain (No MBM). The discovery of the CTC RPL/RPS gene signature of MBM has relevance because variability in ribosomal composition may result in the generation of a “onco-ribosome” which drives increased translation, cell proliferation, and tumorigenesis by means of modulating oncogenic signaling pathways (Li & Wang, 2020; Guimaraes et al., 2016). Enhanced ribosome biogenesis may be critical in achieving metabolic plasticity (Elhamamsy et al., 2022).

Melanoma is the most aggressive skin cancer whose rate of diagnosis is advancing faster than any other cancer type of cancer, due to melanoma's proclivity to metastasize throughout the body. Specifically, MBM significantly reduces overall survival and is linked to poor clinical outcomes, representing a significant biological and clinical challenge (Eroglu et al., 2019; In et al., 2020; Sperduto et al., 2020; Gonzalez et al., 2022; Kircher et al., 2016). One of the fundamental questions still unanswered in the melanoma field is to characterize metastatic-competent CTCs. In contrast to the majority of CTC investigations, a multilevel approach, temporal and spatial, was employed to derive insights for the key CTC properties responsible for overt MBM. It was demonstrated that transcriptional subtyping of melanoma CTCs resulted in the common CTC RPL/RPS gene signature, possibly responsible for MBM onset and progression. It was shown that transcriptional subtyping of CTCs from the Lin− cell population of patients with MBM provided distinct genetic signatures. Meanwhile, CTCs from patients with primary melanoma or patients with melanoma with metastasis to non-brain organs did not share MBM transcriptional profiling. In addition, the first longitudinal CTC transcriptomic analyses of a patient with MBM over a period of 6 months (FIG. 3D) was performed. These transcriptomic analyses were pivotal in identifying the CTC RPL/RPS gene signature of MBM. To further evaluate this signature, additional multilevel studies were performed using MRI CTC-driven mouse model.

Currently, there is a paucity of experimental models of brain metastasis due to inefficient brain colonization, disease latency, and early animal mortality due to metastatic burden in other organs (Eroglu et al., 2019; Gonzalez et al., 2022). Although these models have been an invaluable tool to study MBM, the process by which they have been generated varies greatly from one occurring in patients and involving CTCs. Herein is a report of the establishment of a successful MRI CTC-driven xenograft model of MBM (MRI-MBM CDX model) which mimics human disease development (FIGS. 4 and 5). MRI is a noninvasive imaging technique that has been considered the gold standard for MBM identification, evaluation of clinical brain metastasis, and response to therapy in these settings (Pflugfelder et al., 2013). Importantly, MRI can be used for the longitudinal screening of disease progression within the same individual. The experimental model allowed for the detection and investigation of MBM 24 hours postinjection. This model provides the advantage of performing comprehensive analysis of the multistep process of brain metastasis using a CTC-derived clone (70W-SM3 cells). Longitudinal MRI screening of MBM mice resulted in the identification of specific sites of brain colonization; FL, PTL, and cerebellum, confirming to be major MBM niches as seen by routine radiologic imaging. Detailed transcriptomic analysis of the brain tumors from FL, PTL, and cerebellum was carried out to interrogate MBM-CTC specificity.

A number of recent studies have reported a link between abnormal ribosome synthesis and malignancy formation (Elhamamsy et al., 2022; Li & Wing, 2020; Ebright et al., 2020; Bretones et al., 2018). A study reported that dysregulation of translation in a breast cancer study has been linked to increased metastasis (Ebright et al., 2020). Specifically, increase of RPL15 expression triggered massive metastatic spread to distant organs and induced translation of other core ribosomal subunits. Also, dysregulation in ribosome biogenesis has been linked to increased tumor burden (Elhamamsy et al., 2022). Thus, enhanced expression of ribosomal proteins could potentially result in ribosomopathies associated with MBM development and progression (Elhamamsy et al., 2022; Li & Wang, 2020). Of note, a recent study has demonstrated that increased tumor-specific total mRNA expression (TmS) is observed in 6,580 patient tumors across 15 cancer types and is correlated to disease progression and reduced overall survival. Quantification of cell-type specific total mRNA transcripts can be a prognostic factor in the systemic evaluation of patients to predict cancer progression and clinical outcomes, with TmS expression reported to be an indicator of phenotypic plasticity (Cao et al., 2022). This may be the first study to identify a common CTC RPL/RPS genetic signature of MBM using multilevel analyses that could be used in therapeutic applications.

In synchrony with the above findings and collectively, the present study suggests that the cell translational machine may have another layer of regulation of gene expression refining CTC-associated prognostication. Ribosome biogenesis is a highly coordinated process between RPL/RPS proteins and rRNA assembly factors. This implies a specific vulnerability of CTCs and suggests the targeting of ribosomal biogenesis significantly affects CTC metastatic states. As a way to suppress aggressive CTC subsets which are characterized by high RPL/RPS content, genetic screening of ribosomal protein expression in patients with MBM could potentially be a prognostic factor of the disease severity and outcomes.

The study is based on a limited number of patients with melanoma; therefore, we cannot conclude that all patients with MBM follow these gene pathways and CTC signature. The expected presence of heterogeneity and cancer subtypes among patients adds complexity to drawing definitive conclusions. The animal models had a small sample size and cannot eliminate the possibility of an inherent sampling bias. The possibility that the CTC RPL/RPS gene signature can lead to altered extra ribosomal functions (Shi et al., 2017) cannot be excluded. The study employed a single MBM CTC-derived clone in the majority of the experiments due to the laborious, tedious, and time-consuming work of establishing a MBM CTC clone that successfully recapitulated MBM development and progression in patients with melanoma. Similarly, the longitudinal study was performed on a single MBM patient due to the limited samples availability, patients' consent to these analyses, or patients' poor survival due to MBM diagnosis and progression. There might be additional parallel pathways driving or contributing to MBM that were not detected or evaluated in these analyses. However, the analysis emphasizes the role of RPL/RPS CTC signature in relation to brain metastasis, regardless of cancer type. The RPL/RPS signature of brain metastasis was not observed exclusively in melanoma; 19 RPL/RPS genes of the MBM CTC signature (out of 21) were shared between brain metastasis of melanoma and breast cancer, latter by literature searches of reports investigating brain-homing breast cancer cell lines (Bos et al., 2009). The approach can be viewed as an analysis of MBM using a four-level discrimination to provide a relevant and clinically meaningful gene signature. In conclusion, the identification of the melanoma CTC RPL/RPS gene signature, common to all MBM samples analyzed, can drive the hyperactivation of ribosomal biogenesis and aid MBM onset and progression. These findings provide the conduit for translation to the clinic and set the stage for the development of therapeutic agents to improve melanoma patient care, notably MBM.

### EMBODIMENTS

1. A method to detect in a mammal having or at risk of having melanoma a risk of brain metastasis comprising a) providing a sample from the mammal having circulating tumor cells (CTCs); b) detecting the presence or amount of expression of two or more genes in the CTCs from the sample of a); and c) determining whether the presence or amount in b) is indicative of melanoma brain metastases (MBM).

2. The mammal of embodiment 1, wherein the mammal is a human.

3. The mammal of embodiment 1 or 2, wherein the mammal has melanoma.

4. The mammal of any one of embodiments 1 to 3, wherein the sample is a physiological fluid sample.

5. The mammal of any one of embodiments 1 to 4, wherein the sample is a blood sample.

6. The method of any one of embodiments 1 to 5, wherein the CTCs are human Mel-A⁺ (CD146).

7. The method of any one of embodiments 1 to 6, wherein the CTCs are CD45⁻, CD235⁻, CD34⁻, CD73⁻, CD90⁻, and CD105⁻.

8. The method of any one of embodiments 1 to 7, wherein the presence or amount is increased relative to a corresponding sample from a corresponding mammal without MBM.

9. The method of any one of embodiments 1 to 8, wherein the presence or amount is indicative of onset of MBM.

10. The method of any one of embodiments 1 to 9, wherein the presence or amount is indicative of progression of MBM.

11. The method of any one of embodiments 1 to 10, wherein an increase in expression of at least one of the genes is indicative of MBM.

12. The method of any one of embodiments 1 to 10, wherein at least 3, 4, 5, 6, 7, 8, 9, 10 or more genes are detected.

13. The method of any one of embodiments 1 to 12, wherein a plurality of RPL 12, RPL 13, RPL 18A, RPL 19, RPL 23, RPL 26, RPL 35A, RPL 37, RPL 38, RPL 6, RPL 7, RPL 7A, RPS 12, RPS 15A, RPS 18, RPS 24, RPS 26, RPS 28, RPS 5, RPS 7, or RPS A, or any combination thereof, is detected.

14. The method of any one of embodiments 1 to 12, wherein a plurality of BIRC7, CDH3, CLK1, CSPG4, EIF4B, MRFAP1, PAIP1, PPDPF, RIMKLB, RPL12, RPL13, RPL18A, RPL19, RPL7, RPS12, RPS18, PRS24, PRS26, SPCS2, SPRY4, or any combination thereof, is detected.

15. The method of any one of embodiments 1 to 14, wherein RNA expression is detected.

16. The method of any one of embodiments 1 to 14, wherein protein expression is detected.

17. The method of any one of embodiment 1 to 16, further comprising treating the mammal with a checkpoint inhibitor or a kinase inhibitor.

18. The method of embodiment 17, wherein the inhibitor comprises pembrolizumab, nivolumab, atezolizumab, avelumab, durvalumab, or ipilimumab.

19. The method of any one of embodiments 1 to 18, further comprising treating the mammal with an immunotherapy, stereotactic radiosurgery, surgical resection or whole-body radiotherapy, or any combination thereof.

20. A kit for detecting gene expression comprising probes or primers specific for a plurality of RPL 12, RPL 13, RPL 18A, RPL 19, RPL 23, RPL 26, RPL 35A, RPL 37, RPL 38, RPL 6, RPL 7, RPL 7A, RPS 12, RPS 15A, RPS 18, RPS 24, RPS 26, RPS 28, RPS 5, RPS 7, or RPS A, or any combination thereof; or probes or primers specific for BIRC7, CDH3, CLK1, CSPG4, EIF4B, MRFAP1, PAIP1, PPDPF, RIMKLB, RPL12, RPL13, RPL18A, RPL19, RPL7, RPS12, RPS18, PRS24, PRS26, SPCS2, SPRY4, or any combination thereof.

21. A non-human mammalian model for MBM, wherein the non-human mammal comprises human CTC cells.

22. The non-human mammalian model of embodiment 21, wherein the CTCs are human Mel-A⁺ (CD146).

23. The non-human mammalian model of embodiment 21 or 22, wherein the CTCs are CD45⁻, CD235⁻, CD34⁻, CD73⁻, CD90⁻, and CD105⁻.

24. The non-human mammalian model of any one of embodiments 21 to 23, wherein the CTCs express a plurality of RPL 12, RPL 13, RPL 18A, RPL 19, RPL 23, RPL 26, RPL 35A, RPL 37, RPL 38, RPL 6, RPL 7, RPL 7A, RPS 12, RPS 15A, RPS 18, RPS 24, RPS 26, RPS 28, RPS 5, RPS 7, or RPS A, or any combination thereof.

25. The non-human mammalian model of any one of embodiments 21 to 23, wherein the CTCs express a plurality of BIRC7, CDH3, CLK1, CSPG4, EIF4B, MRFAP1, PAIP1, PPDPF, RIMKLB, RPL12, RPL13, RPL18A, RPL19, RPL7, RPS12, RPS18, PRS24, PRS26, SPCS2, SPRY4, or any combination thereof.

26. A method to prevent, inhibit or treat a mammal having or at risk of melanoma brain metastasis comprising administering to the mammal a therapeutic composition, wherein CTCs in the mammal have increased expression of two or more genes.

27. The method of embodiment 26, wherein the mammal is a human.

28. The method of embodiment 26 or 27, wherein the CTCs have increased expression of a plurality of RPL 12, RPL 13, RPL 18A, RPL 19, RPL 23, RPL 26, RPL 35A, RPL 37, RPL 38, RPL 6, RPL 7, RPL 7A, RPS 12, RPS 15A, RPS 18, RPS 24, RPS 26, RPS 28, RPS 5, RPS 7, or RPS A, or any combination thereof.

29. The method of embodiment 26 or 27, wherein the CTCs have increased expression of a plurality of BIRC7, CDH3, CLK1, CSPG4, EIF4B, MRFAP1, PAIP1, PPDPF, RIMKLB, RPL12, RPL13, RPL18A, RPL19, RPL7, RPS12, RPS18, PRS24, PRS26, SPCS2, SPRY4, or any combination thereof.

### BIBLIOGRAPHY

Alexa & Rahnenfuhrer, Cranio, ______:______2016.
Alix-Panabieres & Pantel, Nat. Rev. Cancer, 14:623 (2014).
Amintas et al., Int. J. Mol. Sci., 21:2653 (2020).
Anders et al., Bioinformatics, 31:166 (2015).
Au et al., Proc. Natl. Acad. Sci. USA, 113:4947 (2016).
Berghoff et al., ESMO Open, 1:e000024 (2016).
Biermann et al., Cell, 185:2591 (2022).
Boral et al., Nat. Commun., 8:196 (2017).
Bos et al., Nature, 459:1005 (2009).
Brayer et al., Cancer Discov., 6:176 (2016).
Bretones et al., Blood, 132:2375 (2018).
Brown et al., PLoS One, 2:e0176675 (2017).
Cao et al., Nat. Biotechnol., [Online ahead of print] (2022).
Chan et al., Pigment Cell Melanoma Res., 30:558 (2017).
Croft et al., ______, 39:D691 (2011).
De Giorgi et al., J. Invest. Dermatol., 130:2440 (2010).
Dianat-Moghadam et al., Cancers, 12:867 (2020).
Dorr et al., Neuroimage, 42:60 (2008).
Ebright et al., Science, 367:1468 (2020).
Elhamamsy et al., Cancer Res., 82:2344 (2022).
Eroglu et al., Pigment Cell Melanoma Res., 32:458 (2019).
Fedorov et al., Magn. Reson. Imaging, 30:1323 (2012).
Fischer et al., Cancer Discov., 9:628 (2019).
Freeman et al., Magn. Reson. Imaging, 16:765 (1998).
Frerich et al., Oncotarget, 9:7341 (2017).
Gonzalez et al., Cell, 185:729 (2022).
Guimaraes & Zavolan, Genome Biol., 17:236 (2016).
Gupta & Massague, Cell, 127:679 (2006).
Hong et al., Proc. Natl. Acad. Sci. USA, 115:2467 (2018).
In et al., Oncotarget, 11:3118 (2020).
Johnson & Young, Neurosurg. Clin. N. Am., 7:337 (1996).
Jones et al., JAMA Surg., 148:456 (2013).
Joosse et al., EMBO Mol. Med., 7:1 (2015).
Khoja et al., Melanoma Res., 24:40 (2014).
Kircher et al., Int. J. Mol. Sci., 17:1468 (2016).
Lee & Wu, Methods Mol. Biol., 750:101 (2011).
Li & Wang, J. Cell. Biol., 219:e202001108 (2020).
Lin & Fisher, Nature, 445:843 (2007).
Lucci et al., Clin. Cancer Res., 26:1886 (2020).
Luke et al., Nat. Rev. Clin. Oncol., 14:463 (2017).
Luo et al., Cell Rep., 7:645 (2014).
Micalizzi et al., Genes Dev., 31:1827 (2017).
Nguyen et al., Cell, 185:563 (2022).
Ordidge et al., Magn. Reson. Med., 16:238 (1990).
Pauken et al., Cancers, 13:4885 (2021).
Pflugfelder et al., J. Dtsch. Dermatol. Ges., 11:563 (2013).
Platt et al., Invest. Radiol., 56:705 (2021).
Shi et al., Mol. Cell., 67:71 (2017).
Sperduto et al., J. Clin. Oncol., 38:3773 (2020).
Sprouse et al., Int. J. Mol. Sci., 20:1916 (2019).
Stocking et ql., Comp. Med., 59:37 (2009).
Thies et al., Br. J. Cancer, 96:609 (2007).
Van derWalt et al., Peer J., 2:e453 (2014).
Vishnoi et al., Cancer Res., 78:5349 (2018).
Werner-Klein et al., Nat. Commun., 9:595 (2018).
Wronski et al., J. Neurosurg., 83:605 (1995).
Zhang & Yu, Cell Biosci., 1:8 (2011).

All publications, patents and patent applications are incorporated herein by reference. While in the foregoing specification, this invention has been described in relation to certain embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A method to detect in a mammal having or at risk of having melanoma a risk of brain metastasis comprising:
a) providing a sample from the mammal having circulating tumor cells (CTCs);
b) detecting the presence or amount of expression of two or more genes in the CTCs from the sample of a); and
c) determining whether the presence or amount in b) is indicative of melanoma brain metastases (MBM).

2. The mammal of claim 1, wherein the mammal has melanoma.

3. The mammal of claim 1, wherein the sample is a physiological fluid sample.

4. The method of claim 1, wherein the CTCs are human Mel-A⁺ (CD146).

5. The method of claim 1, wherein the CTCs are CD45⁻, CD235⁻, CD34⁻, CD73⁻, CD90⁻, and CD105⁻.

6. The method of claim 1, wherein the presence or amount is increased relative to a corresponding sample from a corresponding mammal without MBM.

7. The method of claim 1, wherein the presence or amount is indicative of onset of MBM.

8. The method of claim 1, wherein the presence or amount is indicative of progression of MBM.

9. The method of claim 1, wherein an increase in expression of at least one of the genes is indicative of MBM.

10. The method of claim 1, wherein at least 3, 4, 5, 6, 7, 8, 9, 10 or more genes are detected.

11. The method of claim 1, wherein a plurality of RPL 12, RPL 13, RPL 18A, RPL 19, RPL 23, RPL 26, RPL 35A, RPL 37, RPL 38, RPL 6, RPL 7, RPL 7A, RPS 12, RPS 15A, RPS 18, RPS 24, RPS 26, RPS 28, RPS 5, RPS 7, or RPS A, or any combination thereof, is detected
or wherein a plurality of BIRC7, CDH3, CLK1, CSPG4, EIF4B, MRFAP1, PAIP1, PPDPF, RIMKLB, RPL12, RPL13, RPL18A, RPL19, RPL7, RPS12, RPS18, PRS24, PRS26, SPCS2, SPRY4, or any combination thereof, is detected.

12. The method of claim 1, further comprising treating the mammal with a checkpoint inhibitor or a kinase inhibitor.

13. The method of claim 12, wherein the inhibitor comprises pembrolizumab, nivolumab, atezolizumab, avelumab, durvalumab, or ipilimumab.

14. The method of claim 1, further comprising treating the mammal with an immunotherapy, stereotactic radiosurgery, surgical resection or whole-body radiotherapy, or any combination thereof.

15. A non-human mammalian model for MBM, wherein the non-human mammal comprises human CTC cells.

16. The non-human mammalian model of claim 15, wherein the CTCs are human Mel-A⁺ (CD146).

17. The non-human mammalian model of claim 15, wherein the CTCs are CD45⁻, CD235⁻, CD34⁻, CD73⁻, CD90⁻, and CD105⁻.

18. The non-human mammalian model of claim 15, wherein the CTCs express a plurality of RPL 12, RPL 13, RPL 18A, RPL 19, RPL 23, RPL 26, RPL 35A, RPL 37, RPL 38, RPL 6, RPL 7, RPL 7A, RPS 12, RPS 15A, RPS 18, RPS 24, RPS 26, RPS 28, RPS 5, RPS 7, or RPS A, or any combination thereof
or wherein the CTCs express a plurality of BIRC7, CDH3, CLK1, CSPG4, EIF4B, MRFAP1, PAIP1, PPDPF, RIMKLB, RPL12, RPL13, RPL18A, RPL19, RPL7, RPS12, RPS18, PRS24, PRS26, SPCS2, SPRY4, or any combination thereof.

19. A method to prevent, inhibit or treat a mammal having or at risk of melanoma brain metastasis comprising administering to the mammal a therapeutic composition, wherein CTCs in the mammal have increased expression of two or more genes.

20. The method of claim 19, wherein the CTCs have increased expression of a plurality of RPL 12, RPL 13, RPL 18A, RPL 19, RPL 23, RPL 26, RPL 35A, RPL 37, RPL 38, RPL 6, RPL 7, RPL 7A, RPS 12, RPS 15A, RPS 18, RPS 24, RPS 26, RPS 28, RPS 5, RPS 7, or RPS A, or any combination thereof
or wherein the CTCs have increased expression of a plurality of BIRC7, CDH3, CLK1, CSPG4, EIF4B, MRFAP1, PAIP1, PPDPF, RIMKLB, RPL12, RPL13, RPL18A, RPL19, RPL7, RPS12, RPS18, PRS24, PRS26, SPCS2, SPRY4, or any combination thereof.


SEQUENCE LISTING (62 sequences)

SEQ ID NO: 1 - protein, 165 aa, Homo sapiens [sequence omitted]

SEQ ID NO: 2 - DNA, 634 bp, Homo sapiens
  source 1..634: mol_type = other DNA
cctctcggctttcggctcggaggaggccaaggtgcaacttccttcggtcgtcccgaatccgggttcatccgacaccagccgcctccaccatgccgccgaagttcgaccccaacgagatcaaagtcgtatacctgaggtgcaccggaggtgaagtcggtgccacttctgccctggcccccaagatcggccccctgggtctgtctccaaaaaaagttggtgatgacattgccaaggcaacgggtgactggaagggcctgaggattacagtgaaactgaccattcagaacagacaggcccagattgaggtggtgccttctgcctctgccctgatcatcaaagccctcaaggaaccaccaagagacagaaagaaacagaaaaacattaaacacagtgggaatatcacttttgatgagattgtcaacattgctcgacagatgcggcaccgatccttagccagagaactctctggaaccattaaagagatcctggggactgcccagtcagtgggctgtaatgttgatggccgccatcctcatgacatcatcgatgacatcaacagtggtgctgtggaatgcccagccagttaagcacaaaggaaaacatttcaataaaggatcatttgacaactggtgga

SEQ ID NO: 3 - protein, 211 aa, Homo sapiens [sequence omitted]

SEQ ID NO: 4 - DNA, 2187 bp, Homo sapiens
  source 1..2187: mol_type = other DNA
ctttccgctcggctgttttcctgcgcaggagccgcagggccgtaggcagccatggcgcccagccggaatggcatggtcttgaagccccacttccacaaggactggcagcggcgcgtggccacgtggttcaaccagccggcccgtaagatccgcagacgtaaggcccggcaagccaaggcgcgccgcatcgccccgcgccccgcgtcgggtcccatccggcccatcgtgcgctgccccacggttcggtaccacacgaaggtgcgcgccggccgcggcttcagcctggaggagctcagggtggccggcattcacaagaaggtggcccggaccatcggcatttctgtggatccgaggaggcggaacaagtccacggagtccctgcaggccaacgtgcagcggctgaaggagtaccgctccaaactcatcctcttccccaggaagccctcggcccccaagaagggagacagttctgctgaagaactgaaactggccacccagctgaccggaccggtcatgcccgtccggaacgtctataagaaggagaaagctcgagtcatcactgaggaagagaagaatttcaaagccttcgctagtctccgtatggcccgtgccaacgcccggctcttcggcatacgggcaaaaagagccaaggaagccgcagaacaggatgttgaaaagaaaaaataaagccctcctggggacttggaatcagtcggcagtcatgctgggtctccacgtggtgtgtttcgtgggaacaactgggcctgggatggggcttcactgctgtgacttcctcctgccaggggatttggggctttcttgaaagacagtccaagccctggataatgctttactttctgtgttgaagcactgttggttgtttggttagtgactgatgtaaaacggttttcttgtggggaggttacagaggctgacttcagagtggacttgtgttttttctttttaaagaggcaaggttgggctggtgctcacagctgtaatcccagcactttgaggttggctgggagttcaagaccagcctggccaacatgtcagaactactaaaaataaagaaatcagccatgcttggtgctgcacacttgtagttgcagctcctgggaggcagaggtgagggatcacttaacccaggaggcagaggctgcactgagccaggatcacgccactgcactctagcctgggcaacagtgagactgtctcaaaaaaaaaaaaagagacagggtcttcggcacccaggctggagtacagtgccacaatcatggctcactgcagtcttgaactcatggcctcaagcagtcctccctcagcctcccaagtagaggggtttataggcacgagaccctgcacccaacctagagttgccttttttaagcaaagcagtttctagttaatgtagcatcttggactttggggcgtcattcttaagcttgttgtgcccggtaaccatggtcctcttgctctgattaacccttccttcaatgggcttcttcacccagacaccaaggtatgagatggccctgccaagtgtcggcctctcctgttaaacaaaaacattctaaagccattgttcttgcttcatggacaagaggcagccagagagagtgccagggtgccctggtctgagctggcatccccatgtcttctgtgtccgagggcagcatggtttctcgtgcagtgctcagacacagcctgccctagtcctaccagctcacagcagcacctgctctccttggcagctatggccatgacaaccccagagaagcagcttcagggaccgagtcagattctgttttgtctacatgcctctgccgggtgccggtattgaggcacccagggagctgttactggcgtggaaataggtgatgctgctacctctgctgctgcactcacagccacacttgatacacgatgacaccttgcttgtttggaaacatctaaacatctagtagatgacttgcaggctgttggctaccagtttcctgtctgaggtgtatatgttaacttcgtgatcagtttgtatgtttgggactcttgtcctatgtaaagttaaggtgggccgggtgcagtggctcacgcctgtaatcctaacactgggaggccgaggcgggtggatcacctgatggtgaaacctcatctctactgaaaatacaaaaattagctgagtggtga

SEQ ID NO: 5 - protein, 176 aa, Homo sapiens [sequence omitted]

SEQ ID NO: 6 - DNA, 634 bp, Homo sapiens
  source 1..634: mol_type = other DNA
agaggacacttccttttgcgggtggcggcgaacgcggagagcacgccatgaaggcctcgggcacgctacgagagtacaaggtagtgggtcgctgcctgcccacccccaaatgccacacgccgcccctctaccgcatgcgaatctttgcgcctaatcatgtcgtcgccaagtcccgcttctggtactttgtatctcagttaaagaagatgaagaagtcttcaggggagattgtctactgtgggcaggtgtttgagaagtcccccctgcgggtgaagaacttcgggatctggctgcgctatgactcccggagcggcacccacaacatgtaccgggaataccgggacctgaccaccgcaggcgctgtcacccagtgctaccgagacatgggtgcccggcaccgcgcccgagcccactccattcagatcatgaaggtggaggagatcgcggccagcaagtgccgccggccggctgtcaagcagttccacgactccaagatcaagttcccgctgccccaccgggtcctgcgccgtcagcacaagccacgcttcaccaccaagaggcccaacaccttcttctaggtgcagggccctcgtccgggtgtgccccaaataaactcaggaacgccccggtgctc

SEQ ID NO: 7 - protein, 196 aa, Homo sapiens [sequence omitted]

SEQ ID NO: 8 - DNA, 737 bp, Homo sapiens
  source 1..737: mol_type = other DNA
gcagataatgggaggagccgggcccgagcgagctctttcctttcgctgctgcggccgcagccatgagtatgctcaggcttcagaagaggctcgcctctagtgtcctccgctgtggcaagaagaaggtctggttagaccccaatgagaccaatgaaatcgccaatgccaactcccgtcagcagatccggaagctcatcaaagatgggctgatcatccgcaagcctgtgacggtccattcccgggctcgatgccggaaaaacaccttggcccgccggaagggcaggcacatgggcataggtaagcggaagggtacagccaatgcccgaatgccagagaaggtcacatggatgaggagaatgaggattttgcgccggctgctcagaagataccgtgaatctaagaagatcgatcgccacatgtatcacagcctgtacctgaaggtgaaggggaatgtgttcaaaaacaagcggattctcatggaacacatccacaagctgaaggcagacaaggcccgcaagaagctcctggctgaccaggctgaggcccgcaggtctaagaccaaggaagcacgcaagcgccgtgaagagcgcctccaggccaagaaggaggagatcatcaagactttatccaaggaggaagagaccaagaaataaaacctcccactttgtctgtacatactggcctctgtgattacatagatcagccattaaaataaaacaagccttaatctgccttcc

SEQ ID NO: 9 - protein, 248 aa, Homo sapiens
MEGVEEKKKEVPAVPETLKKKRRNFAELKIKRLRKKFAQKMLRKARRKLIYEKAKHYHKEYRQMYRTEIRMARMARKAGNFYVPAEPKLAFVIRIRGINGVSPKVRKVLQLLRLRQIFNGTFVKLNKASINMLRIVEPYIAWGYPNLKSVNELIYKRGYGKINKKRIALTDNALIARSLGKYGIICMEDLIHEIYTVGKRFKEANNFLWPFKLSSPRGGMKKKTTHFVEGGDAGNREDQINRLIRRMN

SEQ ID NO: 10 - DNA, 1233 bp, Homo sapiens
  source 1..1233: mol_type = other DNA
cctctttttccggctggaaccatggagggtgtagaagagaagaagaaggaggttcctgctgtgccagaaacccttaagaaaaagcgaaggaatttcgcagagctgaagatcaagcgcctgagaaagaagtttgcccaaaagatgcttcgaaaggcaaggaggaagcttatctatgaaaaagcaaagcactatcacaaggaatataggcagatgtacagaactgaaattcgaatggcgaggatggcaagaaaagctggcaacttctatgtacctgcagaacccaaattggcgtttgtcatcagaatcagaggtatcaatggagtgagcccaaaggttcgaaaggtgttgcagcttcttcgccttcgtcaaatcttcaatggaacctttgtgaagctcaacaaggcttcgattaacatgctgaggattgtagagccatatattgcatgggggtaccccaatctgaagtcagtaaatgaactaatctacaagcgtggttatggcaaaatcaataagaagcgaattgctttgacagataacgctttgattgctcgatctcttggtaaatacggcatcatctgcatggaggatttgattcatgagatctatactgttggaaaacgcttcaaagaggcaaataacttcctgtggcccttcaaattgtcttctccacgaggtggaatgaagaaaaagaccacccattttgtagaaggtggagatgctggcaacagggaggaccagatcaacaggcttattagaagaatgaactaaggtgtctaccatgattatttttctaagctggttggttaataaacagtacctgctctcaaattgaaatatattgttgtatttgtgatttgttgttgttgttgttagcctgcctctgtcttcccttaactactgtggcaagtgtggtgtgtgataaaattaagccaaatcaacagcccatttcgtgcaaaatcagggtcgagtctgtgtgaaagacatctcttgggtttttaaaaggcttttctatacaaaagattttaattttttgttttttaactgtgctgagtgattcgaaatgggtttattctaagaaagcctgtttcacaagcatttgtacatgatttgtcggtaaggtaatccaactttggtttatggaaaaaattgaatttagttgctaaaatttatttctcgggccttcgcttgtttataaactgccatgtaaatgtgttgttttccttttgcattcca

SEQ ID NO: 11 - protein, 132 aa, Homo sapiens [sequence omitted]

SEQ ID NO: 12 - DNA, 503 bp, Homo sapiens
  source 1..503: mol_type = other DNA
ctctttccctgccgccgccgagtcgcgcggaggcggaggcttgggtgcgttcaagattcaacttcacccgtaacccaccgccatggccgaggaaggcattgctgctggaggtgtaatggacgttaatactgctttacaagaggttctgaagactgccctcatccacgatggcctagcacgtggaattcgcgaagctgccaaagccttagacaagcgccaagcccatctttgtgtgcttgcatccaactgtgatgagcctatgtatgtcaagttggtggaggccctttgtgctgaacaccaaatcaacctaattaaggttgatgacaacaagaaactaggagaatgggtaggcctttgtaaaattgacagagaggggaaaccccgtaaagtggttggttgcagttgtgtagtagttaaggactatggcaaggagtctcaggccaaggatgtcattgaagagtatttcaaatgcaagaaatgaagaaataaatctttggctcaca

SEQ ID NO: 13 - protein, 152 aa, Homo sapiens [sequence omitted]

SEQ ID NO: 14 - DNA, 549 bp, Homo sapiens
  source 1..549: mol_type = other DNA
ctctcttccacaggaggcctacacgccgccgcttgtgctgcagccatgtctctagtgatccctgaaaagttccagcatattttgcgagtactcaacaccaacatcgatgggcggcggaaaatagcctttgccatcactgccattaagggtgtgggccgaagatatgctcatgtggtgttgaggaaagcagacattgacctcaccaagagggcgggagaactcactgaggatgaggtggaacgtgtgatcaccattatgcagaatccacgccagtacaagatcccagactggttcttgaacagacagaaggatgtaaaggatggaaaatacagccaggtcctagccaatggtctggacaacaagctccgtgaagacctggagcgactgaagaagattcgggcccatagagggctgcgtcacttctggggccttcgtgtccgaggccagcacaccaagaccactggccgccgtggccgcaccgtgggtgtgtccaagaagaaataagtctgtaggccttgtctgttaataaatagtttatatacctatggc

SEQ ID NO: 15 - protein, 130 aa, Homo sapiens
MNDTVTIRTRKFMTNRLLQRKQMVIDVLHPGKATVPKTEIREKLAKMYKTTPDVIFVFGFRTHFGGGKTTGFGMIYDSLDYAKKNEPKHRLARHGLYEKKKTSRKQRKERKNRMKKVRGTAKANVGAGKK

SEQ ID NO: 16 - DNA, 534 bp, Homo sapiens
  source 1..534: mol_type = other DNA
ctcttttcctccttggctgtctgaagatagatcgccatcatgaacgacaccgtaactatccgcactagaaagttcatgaccaaccgactacttcagaggaaacaaatggtcattgatgtccttcaccccgggaaggcgacagtgcctaagacagaaattcgggaaaaactagccaaaatgtacaagaccacaccggatgtcatctttgtatttggattcagaactcattttggtggtggcaagacaactggctttggcatgatttatgattccctggattatgcaaagaaaaatgaacccaaacatagacttgcaagacatggcctgtatgagaagaaaaagacctcaagaaagcaacgaaaggaacgcaagaacagaatgaagaaagtcagggggactgcaaaggccaatgttggtgctggcaaaaagtgagctggagattggatcacagccgaaggagtaaaggtgctgcaatgatgttagctgtggccactgtggatttttcgcaagaacattaataaactaaaaacttca

SEQ ID NO: 17 - protein, 115 aa, Homo sapiens [sequence omitted]

SEQ ID NO: 18 - DNA, 1140 bp, Homo sapiens
  source 1..1140: mol_type = other DNA
gattttcttccgccatccggctaaatagtcccatgtgcactttgttccatggataaataaacactaggaacgcatttccaccctagatttcagcagaaatgctgaatgtaaaggaatatttgagtaaagtgagttgccgttcttgaagcccgtctcctaaggattctcccggtgtccgcgtagggatctcatgctatataggagggccctgccaggcaccgtctcctctctccggtccgtgcctccaagatgacaaagaaaagaaggaacaatggtcgtgccaaaaagggccgcggccacgtgcagcctattcgctgcactaactgtgcccgatgcgtgcccaaggacaaggccattaagaaattcgtcattcgaaacatagtggaggccgcagcagtcagggacatttctgaagcgagcgtcttcgatgcctatgtgcttcccaagctgtatgtgaagctacattactgtgtgagttgtgcaattcacagcaaagtagtcaggaatcgatctcgtgaagcccgcaaggaccgaacacccccaccccgatttagacctgcgggtgctgccccacgtcccccaccaaagcccatgtaaggagctgagttcttaaagactgaagacaggctattctctggagaaaaataaaatggaaattgtacttaatattgcatgttaagtgtatctgtgccagataaggtggggattttgtgtgttagaccaagtgtgaagtgacacacattattttcatggggaagaaagcttattcatgtaatttaatttttttcttttttttttttttttttttgagacggagtctttgtcgcccaagctgaattgcagtggcgtgatctcagctcactgcaacctccgtctcccgggttcaagtgattctcctgcctcagcttcttgagtagctgggattacaggtgtctgccaccatgcctggttaatttttgtatttttggtagagatggggtttcactatgttgtccaggctggtcttgaacttctgacctcagttaatccaccagccttggcctcccaaagtgctgggattacaggcttgagccacctcgcctagctatttatgtaaattaaactttaattgtggtcgtatggttggcctcacaattcgca

SEQ ID NO: 19 - protein, 140 aa, Homo sapiens [sequence omitted]

SEQ ID NO: 20 - DNA, 2706 bp, Homo sapiens
  source 1..2706: mol_type = other DNA
cggcgttcaagatgtcgaagcgaggacgtggtgggtcctctggtgcgaaattccggatttccttgggtcttccggtaggagctgtaatcaattgtgctgacaacacaggagccaaaaacctgtatatcatctccgtgaaggggatcaagggacggctgaacagacttcccgctgctggtgtgggtgacatggtgatggccacagtcaagaaaggcaaaccagagctcagaaaaaaggtacatccagcagtggtcattcgacaacgaaagtcataccgtagaaaagatggcgtgtttctttattttgaagataatgcaggagtcatagtgaacaataaaggcgagatgaaaggttctgccattacaggaccagtagcaaaggagtgtgcagacttgtggccccggattgcatccaatgctggcagcattgcatgattctccagtatatttgtaaaaaataaaaaaaaaaactaaacccattaaaaagtatttgtttgcagtgcttgtctccctgttcacttccagggttcaagcgattctcctgcctcagcctcctaagtagctgggattacaggcacctgccgtcatgcctggctagtttttgtatttttggtggagacagggtttcaccatgtttgccaaacgactcaaactcctgatctcaagtgatctgcccacccaggcctctacagtgtttttgacataccctgaccatcacttttctgaaatggaaactctgggcattttttttttaaagcaatccttgcttttttgtgagtttgcagactttcagcatcttccaattgctgtatttacaattttggcctcaaaaagtattatttgggttttgagtcccaaaaataatagtagatactgctcaatgactggctatacatggtagcttctcctggagtgaggaaggcattcaaatggctgggcgcggtagctggcacctgtaatcccaacactttcggaggccaaggcaggcagatcatgaggtcaggagttcaagaccaggctgaccaacgtggtgaaacctcctcttttctaaaaatacaaaaattagctgggcgtggtggcgcaccgcctgtaatctcagctactgagagaatcgcttgaacctgggaggtggagtttgcagcgagtcgagattgcaccattgtactccatccagcctgggcaacggagcaagattccgttcccgcccccgccccaaaaaaaggcattgaagttaagatagactatatagttagatcctggacacacacatggattttgaggaatgatgtgagtttgtttatgtatgtacattttagcagttaacagatttggagtaaattgaatatttataaaacaacagtaattgccatgtaggtttactgtcatagtggaagatgattaagttgatggtacctacaggtattttgctatgaaaatgttttgacaaacaggatgatcgtgtaatttatgttccaagctctagtttgagaatggaagaatgtggtaaatttttgccaactgaacaggcataaagcgctgataataagggacttggccttaaggtaggaggttgttagcatttctttctaaacgtgtaagagtttatttaggtgacacccagcgttttggaaaaatgggtgtttgtttagaacaataatttggagggaagtggactaaacagggtttttagattaaggtttgtgtttatgtatctgtatctgcaaatactcagccataaatgtttctacctgtaagttgggtataatacaagctccacttggtatcaaaaaggactacccttagtgtcttccatgactagttatggaatgtaccagacctagagaggagttgttctaacctggagcttttgaaaatgtttccggtccataccctagaccaattaagtcagactgcagaataggactcagacatcagaattgtgaagctcccaggagatgtcaaggtatacccaagactgagaaccagtaccctgtctagactgaaccaggcttggttttagaagtattaaatctcgcctgggtacggtggctcacacctataatcccagcactttgggaggctgaggtgggtggatcatctgaggtcaggagagtgagaccagcctgtccaacatgttgaaaccccatctctactatacaaaattagccaggcatggtggtgcatgctgtaatcccagctacttgggaggctgaggcaggagaattgcttgaaccccggagatggaggttgcagtgagctgaggtcgcacgattgtgctccagcctgggcaaccagtgaaactctggggggaaagaaaaaaaatgtattaaatctctagttttagagagttacaccataacatccctgagtatggtcaatttcaagtcagctttagtgtgacaagcccctaggcccaccatttatgtctttatatattatggcaatatatgatccttatatattaaccactaatcgctgctgtttttgaatgttatcttttctgaggcagtcttgctctgtcacacaggctggagtgcagtaacaccactgaagctcactgtaactttgaactgctggacttggaatcctcctgctgtggccttcaaagggctgcaattacaagtgtgagccactgcatcccacctcacattttattctttggagattttttttgacttggattaaaaaactttatatttacacttca

SEQ ID NO: 21 - protein, 145 aa, Homo sapiens [sequence omitted]

SEQ ID NO: 22 - DNA, 408 bp, Homo sapiens
  source 1..408: mol_type = other DNA
ctcttcccttttgcggccatcaccgaagcgggagcggccaaaatgaagtttaatccctttgtgacttccgaccgaagcaagaatcgcaaaaggcatttcaatgcaccttcccacattcgaaggaagattatgtcttcccctctttccaaagagctgagacagaagtacaacgtgcgatccatgcccatccgaaaggatgatgaagttcaggttgtacgtggacactataaaggtcagcaaattggcaaagtagtccaggtttacaggaagaaatatgttatctacattgaacgggtgcagcgggaaaaggctaatggcacaactgtccacgtaggcattcaccccagcaaggtggttatcagtaatcttatatacaagctttgattaaaacttgaaacaaagagcctg

SEQ ID NO: 23 - protein, 110 aa, Homo sapiens [sequence omitted]

SEQ ID NO: 24 - DNA, 1234 bp, Homo sapiens
  source 1..1234: mol_type = other DNA
cttctcttaccgccatcttggctcctgtggaggcctgctgggaacgggacttctaaaaggaactatgtctggaaggctgtggtccaaggccatttttgctggctataagcggggtctccggaaccaaagggagcacacagctcttcttaaaattgaaggtgtttacgcccgagatgaaacagaattctatttgggcaagagatgcgcttatgtatataaagcaaagaacaacacagtcactcctggcggcaaaccaaacaaaaccagagtcatctggggaaaagtaactcgggcccatggaaacagtggcatggttcgtgccaaattccgaagcaatcttcctgctaaggccattggacacagaatccgagtgatgctgtacccctcaaggatttaaactaacgaaaaatcaataaataaatgtggatttgtgctcttgtatttttaagtggattaaaaaacttactaccttaaattgatttgctacatgcttaaaatgatagaggttgctcagcatttttggagtacaagggggtcagagagacatgtgatgaaaattacagggcgagtacagagatttagaagggaacgggttttaatgcgagtatctttgacagagtcttgctctgttgcccatgctggagtgtagtggtgctcgctgcagcctcacattcaaaggctcaagcaatcctcccttggcctttgaagtagctgggaccacaggctcatgccaccatccctgggtcatttttaaattttttgtagagagggtctgactcttgcctatgctggcttcaaactcctgggctcaagcaatcctccttccttggcctctcctgaagtgctgggatacagttatgagccaccacacctgccaagtgctttgtgatactatgcatttgttcaatgcagattgggaaacttaaaatttgaatggagattatgttgatgggctttggcagttcatttggatagactgggatgagaagctcttgggacttgtgactggacaaagcattccagtatattaaaataaaattaagccatattactccactcataaaaagcaatcctatggtaggtacatggaggttgggaatagtgcacggaaaggtggcagctttctttggcttcatgttttaatctggtaaagttcaagattgcactttaagcaggcctcctaaatattttagatttcttggggatatgctaaaataaaacaactaaggcatca

SEQ ID NO: 25 - protein, 97 aa, Homo sapiens [sequence omitted]

SEQ ID NO: 26 - DNA, 7573 bp, Homo sapiens
  source 1..7573: mol_type = other DNA
ctcttccggtctttctggtctcggccgcagaagcgagatgacgaagggaacgtcatcgtttggaaagcgtcgcaataagacgcacacgttgtgccgccgctgtggctctaaggcctaccaccttcagaagtcgacctgtggcaaatgtggctaccctgccaagcgcaagagaaagtataactggagtgccaaggctaaaagacgaaataccaccggaactggtcgaatgaggcacctaaaaattgtataccgcagattcaggcatggattccgtgaaggaacaacacctaaacccaagagggcagctgttgcagcatccagttcatcttaagaatgtcaacgattagtcatgcaataaatgttctggttttaaaaaatacatatctggttttggtaaggtatttttaatcaattaggcttgtagtatcagtgaaatactgtaggtttagggactgggctagcttcatatcagatttacttgttaagtgactgttttggaatgtttacttttggactgggtttgtaacacggttaaaggcaatgagaaacaagcagaattccaggagtccttgaagcagagggcactggaagacaatatagcagattaaaatagcacagctcatgtggcataggtgggtattttagatgtttgagtaaatttgaaagagtatgatgtttaaattacctttagcaacatgttcatctgctatgctgtcatgactagggggatgattattagtcacatagagcttgggagtaccactggaaacgtatgggtaggagtttaggtggcttctgtttttcaaaagatgatcttatcctagtatctgtaatgctcacttggcacacctgacttgtgggctgtgtgtaaggtggctagctaagtgaaaaaagcctgctaggtgtgagtcaacttaagaatatgtaaataggtttgagaaaaagtagggcttgggtgcaagtaaagattgagcaggaaataaaggaaaatcaagtataatccctgagatttgtagactaaaggcaatgatgtgggactacttggtcgaatttttttagccctcaacttggtaattgggtgtttctgtgttaaagcactgaaacttgctgtcgtgccttcctagttttcgtggtttattgacagggttgggggttttttttgtttttttaaaatgaagggacaaagtcaactggactgctgagtgagagggcaggggcagttgaagggaacatgaattgctggaacagctacataaaatagtgatgtagccaagtcatgctatttaaattataattctccactgtgtttagaataacatctgaggttcttaacctggccttggaagggtatcacttttacttgtaacctggaatggctttataatgtgctagctaattgctactctcatcttgtattttaactcctaatttacccttcaggtctcagcttcagaacattcacttataaagaaaccctgctgattaaatctctcttgggcttcctcccgaaatgtgagactatactttaaagatgtatggttagagtccaattgccattgcctttcttgtttacagatagtgcaatggcgcaatcctggctcactgcagcctctaattcctgggctcaagtcaagtggttctcctgcctcagccttctgagtagctgggactacagatgcacaccaccacacctggctaatttttgtgttttttgtagagatggagattcactcgactaattctttttgtattcttagtagagactgggtttcaacatgttgaccaggctggtctcgaactcgacctcgtgatccgccgcctcggcctcccaaagtgctaggattacaggcgtgagccaccactcccggctgtcatcatcaaattttcaagtgaagatagtctgttgaagattgaacaatgaccttgaaagacagctgagttgctgtgggtataatgtaaagctggtgaagtttggccagtttgggcttcagaagtctaagtctagtgaaggtaccctgacccccatataaacaacccttgaggccggtgtggtggctcacggctgtaatcctggcactttgggaggccgaggtgggcggatcacaaggtcaagagatcgagatcaccctggcccacatggtgagaccccaactctactaaaaatacaaaaattagctgggtgtggtggcatgtgcctgtagtcttagctacttgagaggctgaggcaggagaatcgcttgaacctgggaggcagaggttgcagtgagctgagatcatgccactgtgctccagcctggtggcacagtgagactccacctcaaaaaaaaaaaaaaatccttgaactaatgactcaaattttcaaatgaaacaaaataagcagtggatcttgcattggagatggagttaaactatgttgcccaggctggtcttggactcctggtttcaagtgatcctctcatcttggcctcccaaagtgctgggattacagggacgagccaccacaccccacctattgtctatatttctatctttaacagcacttcagtcctgccttaagttatagttatgtatagatacccattatactttaaatttttcagcagaaattatgcttttatcttctctgcagtgttatatgttggtgtgcaaaaatgttaaatttatttttcctaagtaccccatctgcttttcaactctgtcctctgcctgaaaagcctccctccagcccctacttccctcccatcttagttcacaaagtcaggttgatttgcccccagctgtcaaagcagactacctgtttccacatgtaactggttatgttctgtaaagttacaaaaatagaaaggttgaatctgtggcggccgggtacggtagctcacgtcctatagtcgcagcactttgggaggccaaggtgggcagttcacgtgaggtggggagttcgagaccagcctgaccaacatggagaaaccccgtctctactaaaaatacaaaaatattagccgggcgtggtggtgcatgcctgtaatcccagctactagggaggctgaggcaggagaattgcttgaacctgggagacggaggttgcggtgagctgcagaggcctcacctctaattgagacacaattatatattgttgatatatatatatatatatatacacacacacacacacactatgatggataaatgcatgagtttctgtgagagcattggaaaggagtttgtcactcaataggtgaagccaggctaagatttaagctgagccagggaggacttgaaggaatcatgatgagagagaaggtaagtggctttgccagcaatgaaacagctgacataatggtaaccagtcagaggagggcataactatgaaactggacaccttggttgtcaggttagaaggatggggtgtagggttggtaagaaaagaattcagggaagagcagcgatcagattatgaagaatttgtcttgagaaattacagaggatttaaaccagaatgttaggaatagttattctagcaagatgaatgtggaaagtgttagtgtgcatgtgatgagtcttgaagctggaaactaggtaacaggttcttaaatagttcatgtgaaaatcatgacagactaaggcagtggctgtggggctgtccgggagttctctacagaaaacatctaaaacttgaatgtgcaagtgagtagctaacttccaagcttcccatttctgtataatttaagcatgaaaatgagaacactgagatttgataggcatgtagaagtcagagtaagcaagagggcttgagttcatcgatgaacttcagtaactatccttgacttagtattggtggaaaccatttgtgaatttacagactccaaaaacaaaaagataatttagagtctcaaagtaagatttggggagatgtccattggagacagaagtggagagagggagaagttcatttgttgacatatttatgtatggtgcacctactgtgtgccaagccatgttagagatacagtgggagcaaaaccagatgtgttttctgccctctgactccagtggttgcagaattagcccaggaactagaattggatggagtcacacaaaccaaggaaggggattgtttgaggggggcactcagtcacatgtggctgaggccaaagagaatgaagatatattttaaaattctggatttggcaaaatttaggctagtctttacttttcataaacctctctcatgttagagcagcaaagaaaaaccagtttgccaagaggtcacgagattgggcagtgaggaatggggagtttagtttaaaagacaacactagatacttttggaagtttacctattctgtggttcccttttattgactaaatggtgcccttcattcatgaagcaagtatctatttagtgactactatgtgtcaaaagctatctaggcccagaaacttttaagtggaaaacaaaacaaagaccctgccctgacagatttcaatcatgtatatactgtatgtatgtttgtggaaggtgacataatagacaattgcagatagtgataagtgtgtaagacaaggataccacgagcgtaatagacagtggggatggaggaacctttagaaggttggttaagttgaatgataaggagccattcttttgtctttacctgaatgacttatcattcagccctttttgagttttgggttgcttgcagagtttaaccttgcctgtaattgaaacttaattttgtaatagcataacttcatgtaagaaaagcaaagcaatacattacagaattattttactgaagaacttgtttcagagaaagaggctgtttcacatttattagcacatttggattatatttaggtgttttatttttttttaaacaaaggagtttggatcataatacaagagaagcacagggcaaagacactgcataacctcaagaactaagaatggaaggactggccaggcctggtggtgcacacctgtaatcccagctactcaggaggctgaggcgagagaatcgcttgagcctggggggcagaggttgcagtgagctgagagaacggcgggcggcagagcgagactccatctcaaaaaaaaaaaaggataaaggaaggacttaagcaaaatcttccttgtaagtagaaggatgttttgacaagaaaagttgcaatggaaaaatggttctcatgtacacgagtatgtagaataagcatcgtgtgtggattggattcagatcaaaacattgcttttatgtttgtgtctttatacggtgggagtataccctggtgccccaggatgaagacttgacctgacccatgtatttttagattactcacagataacaaaaagtattttcatcatgattagttgcgaaaacagttttatttcaataggtaaaacgtgcagtcctatgtaatcgtcagaaggtaatcttaattatagcttgggtgtgctttaaactgcaagctggcagtggagggcacgattcctctgatttcagctttctccttatacttttctggagctgtgagctgcaagttaactcagtgggattaaagtgtagactggaggtacaaaaggtgaggagtgaggagatagggtagttcttccttggctggctggcttcataatccctgggccccgcagataattaaatcgactttttctgtctcaggcatttgtatgacctctttggaggttccctgctgggtagttatccttgtatctgatggacccatctcaatttaaaatactctgccaggttcggaggttcatgcttgtcatcccagcactttgggaggctcagaggtgccattggcttgagcccaagagtttgagaccagcctgggcaacctggtgaaacctcttctccattaaaaatacaaaaaattagccaggcatggtggtatgtgcctgtagtcccagctactcagggagctgaggtggaaggattgcttgagcctgggaggtcaaggctgcagtgaaccttgattgtgccactgcactccagcctggatgacaaagaccctgtctcaagaaaagtctgtaattctttcctaacccttagtatccagcctcagtcctgaggttttctttacctctgggggctattttatgccggctctctcctgagtgtcacacatctggtcctcaggaaacattctcacatccctggcctgaaaaaaacaatttcagggagatcgcatggcagcagccctctctgggctccccagctaaatggttgtacgaaacacatttcaaagctctctgaagggcttccttgaagttcctttcactgggtttcaaagtagaaggtagtaactcctttgtccaagaaggctgaattgagcacttaacaattctccaaagaaatttcttcattggtctctatcctagaccccttctgtatccttgatatggctgaggattctaaaaaatgaccagtcttacatgggagggctgggatataaaaaataaaataataaataatgataaaaactgaaaactgaccacgttcttggatatgttttctttgggttgtgtgtgtgtgtgagacttttgatagttacccaaagtaggaaaaatcccattctaataaggttatatttatgtagctctgcaaataaacatctagcaaatgtaaaaagtattttctttgccttaaaaatgattaaaattatttgaactcctgaggagtgttatatgaataaaattagtaagttatttggaggaaagttattttttaaaaagacaactggtaaaacagtacaggagaaaggccagcttcctcaagtgaggacagttgtttagaattgactgaggagcggccgggtgcggaggctcacatctgtaatcccacacgccttgggaggctctgaggcgggtagatcacctgaggtcaggagtttgagaccagcctggccaacatggcgaaaccccgtctccactaaaaatacaaaaattagccaggtgcagtggcacacacctgtaatcccagctactccgaaggctgaggcaggaggatcacctgagcccaggaagttgagactgcagtgagctgagattgcaccactgcactccagcctcagtgacagcgagactgtctcaaaaaaagaaaaaagtgactgaggaggaagaggccaggtggcaaatggaacagaatcaccaaagggtgaacaggactaaggcaatgtagtgtatggctcagctacgtcagagtggaaaaggtgttattagagcagaaactatggtccctgcgtcacagggaagcaacctacagagaagcagcagctccccaagagaggagagataagaagccagaagcctcagagtgaacaattgtcctattagggattgctctagagagagaaacctctgggaacgtacccctgtgaggcagcacagcacaatgcttttagaattgtatgagagttgatatatctccatttgttttgcaaaggcaaaaactaagatacagagattatctgttaaagttatgtatttctttggtaataaagatgctgacagtgttgctggaatgcatttctttaataaagatattgtacaatgaaa

SEQ ID NO: 27 - protein, 70 aa, Homo sapiens [sequence omitted]

SEQ ID NO: 28 - DNA, 1145 bp, Homo sapiens
  source 1..1145: mol_type = other DNA
ctttttcgtccttttccccggttgctgcttgctgtgagtgtctctagggtgatacgtgggtgagaaaggtcctggtccgcgccagagcccagcgcgcctcgtcgccatgcctcggaaaattgaggaaatcaaggacttcctgctcacagcccgacgaaaggatgccaaatctgtcaagatcaagaaaaataaggacaacgtgaagtttaaagttcgatgcagcagatacctttacaccctggtcatcactgacaaagagaaggcagagaaactgaagcagtccctgccccccggtttggcagtgaaggaactgaaatgaaccagacacactgattggaactgtattatattaaaatactaaaaatcctaagtgtctttcgtctttgcggatgggaaagggaaaaatgctacctcgtagtggcttctgatgggaacaggacgcgggttctgttgctgccttcctgtgtctttttttttttttttttttctttctttgagacggagtcttgctctgtggctcatcctggagcacagtggtgcgatatcagctcactaccacctccgcctcctgggttcaagcgactgtcctgcctcagcctcccgagtggctgggattacaggcacacatcaccacgcctggccaatttttgtatttgtagtagagacagggtttcactgcctgcctcagcctcccatagtgctgggattacaggcatgagcctccgtgcccggtgcatccctaatcttgagcatgatctcagtcggcaaatgaggccatctgttttcagcctgtttgaaaataagatgtggggaggccatgatggaaatagcacgtggggttaaacataactggcagatgtgggagcgatggtggggcatgccattcaaacaggtcccaaaatgggtgcaacaaggtatagcacatctaccactcgctaacttgactgacttggagaaatgactacacttttgcctgtttcctcagttggaaaatagccatattaacacctctttcattggcttgctgtcagggtactgggatggggggaggtgcatgggttggggtggccaccaggtggtgctgtgccacagcgggcagcccctctggaaatgactggcatcataaaatctgtcttcatacccga

SEQ ID NO: 29 - protein, 288 aa, Homo sapiens [sequence omitted]

SEQ ID NO: 30 - DNA, 921 bp, Homo sapiens
  source 1..921: mol_type = other DNA
ctctttcccatcttgcaagatggcgggtgaaaaagttgagaagccagatactaaagagaagaaacccgaagccaagaaggttgatgctggtggcaaggtgaaaaagggtaacctcaaagctaaaaagcccaagaaggggaagccccattgcagccgcaaccctgtccttgtcagaggaattggcaggtattcccgatctgccatgtattccagaaaggccatgtacaagaggaagtactcagccgctaaatccaaggttgaaaagaaaaagaaggagaaggttctcgcaactgttacaaaaccagttggtggtgacaagaacggcggtacccgggtggttaaacttcgcaaaatgcctagatattatcctactgaagatgtgcctcgaaagctgttgagccacggcaaaaaacccttcagtcagcacgtgagaaaactgcgagccagcattacccccgggaccattctgatcatcctcactggacgccacaggggcaagagggtggttttcctgaagcagctggctagtggcttattacttgtgactggacctctggtcctcaatcgagttcctctacgaagaacacaccagaaatttgtcattgccacttcaaccaaaatcgatatcagcaatgtaaaaatcccaaaacatcttactgatgcttacttcaagaagaagaagctgcggaagcccagacaccaggaaggtgagatcttcgacacagaaaaagagaaatatgagattacggagcagcgcaagattgatcagaaagctgtggactcacaaattttaccaaaaatcaaagctattcctcagctccagggctacctgcgatctgtgtttgctctgacgaatggaatttatcctcacaaattggtgttctaaatgtcttaagaacctaattaaatagctgactacat

SEQ ID NO: 31 - protein, 266 aa, Homo sapiens [sequence omitted]

SEQ ID NO: 32 - DNA, 887 bp, Homo sapiens
  source 1..887: mol_type = other DNA
ctttctctctcctcccgccgcccaagatgccgaaaggaaagaaggccaagggaaagaaggtggctccggccccagctgtcgtgaagaagcaggaggctaagaaagtggtgaatcccctgtttgagaaaaggcctaagaattttggcattggacaggacatccagcccaaaagagacctcacccgctttgtgaaatggccccgctatatcaggttgcagcggcagagagccatcctctataagcggctgaaagtgcctcctgcgattaaccagttcacccaggccctggaccgccaaacagctactcagctgcttaagctggcccacaagtacagaccagagacaaagcaagagaagaagcagagactgttggcccgggccgagaagaaggctgctggcaaaggggacgtcccaacgaagagaccacctgtccttcgagcaggagttaacaccgtcaccaccttggtggagaacaagaaagctcagctggtggtgattgcacacgacgtggatcccatcgagctggttgtcttcttgcctgccctgtgtcgtaaaatgggggtcccttactgcattatcaagggaaaggcaagactgggacgtctagtccacaggaagacctgcaccactgtcgccttcacacaggtgaactcggaagacaaaggcgctttggctaagctggtggaagctatcaggaccaattacaatgacagatacgatgagatccgccgtcactggggtggcaatgtcctgggtcctaagtctgtggctcgtatcgccaagctcgaaaaggcaaaggctaaagaacttgccactaaactgggttaaatgtacactgttgagttttctgtacataaaaataattgaaataatacaaattttccttca

SEQ ID NO: 33 - protein, 130 aa, Homo sapiens [sequence omitted]

SEQ ID NO: 34 - DNA, 2137 bp, Homo sapiens
  source 1..2137: mol_type = other DNA
ctctttccgccatctttccgcgccgccacaatggtgcgcatgaatgtcctggcagatgctctcaagagtatcaacaatgccgaaaagagaggcaaacgccaggtgcttattaggccgtgctccaaagtcatcgtccggtttctcactgtgatgatgaagcatggttacattggcgaatttgaaatcattgatgaccacagagctgggaaaattgttgtgaacctcacaggcaggctaaacaagtgtggggtgatcagccccagatttgacgtgcaactcaaagacctggaaaaatggcagaataatctgcttccatcccgccagtttggtttcattgtactgacaacctcagctggcatcatggaccatgaagaagcaagacgaaaacacacaggagggaaaatcctgggattctttttctagggatgtaatacatatatttacaaataaaatgcctcatggactctggtgcttccacttggtcgttttgagcctttacagcagtgtagccacagcttctgcggcagcatgcagttgcttcgtttatcggtgaatgcgattccctgaagtgactaatacagccaagggaaaaagttcttatgaaaccagtatgcctaagaaacagtcacccctgctgtctgccaaaaccaggtatttgacactaaatattttagttgtatttcagtttttttttttttttttcttttttggagacagagtctgactctattgctcaggctggagtgcagtggcgcgatcttggcccactgcaacctccacctcccgggttcaagtgattgtcctgtctcagccgcctgagtaactgggattacaagtgtgtgtcgccacatccggctaatttttatattttagtagagacagggtttcgccatgttgcccaggctggtcttgagttctgggcctcaagtgatcagcctacctcggcctcccgaagtgctgggattacagccacgagccattacacctggcctatatttcagtattttctattagtttttgatgaatttgttttgcctggctaggattattctgtagataggattttagatctggcttttgtcactgactgctgtaataaatacttgctaggaatttttttttttttttttttttttttaagacaaagtcgctctgtcacacaggctggagtgcagtggcatgatcttggctcactgcacctccgcttcccagattcaagtagttctcgtgcctcagcctcctgagtagctgggattacaggtgtgtgccaccatgtctggctgatttttgtatttttagtagaggtggggtttcaccatgttggccaggctggtctcgaactcctgatctcaagtgatctgcctgccttggtctcccaaagtgctgggattacaggtgtgaccaccacgccctgccttaagaattgttccaagagaatctggtgccacttgcaggtgcccattgaagtgcaatgggcactgttgatcactgaggaggtagtgggtgctgacccggtgctggggcctgtcccctagtctctgctttgcccttggctagctaggtggtgtgccaagtggggagagaagctaccttattaaggggcatggatcagcttcctgaaaggagggcctgcctctgtaagatatgggaagtcgctgagaatgttacagaacggccctagagatggggcagataacggcccccatttgtgagaagtgagttgggaggcatgtttggggcctctgatgtttgggaggctgtgggtaattaacatgagttttggggtccagcagcagaattcaggtttcctcttccactcagtaacctcagcatccgtatctgtaatgggaatgatacaaaacctatccccaagttgagggaaaaatgagattgtgtaaagcgcacttggcacatgacagtcacaagcatggggacagtgagtccagaaggattttcttatgccagcattgtaagccctaggatcacagggctctggcttgtttaaccatcgtgtctctggtccctagcctgcaaacctggtgtgtagggatgcctcagtcgcttacatgttgattgagtgaatcgtcggtttctttctggacactgacttcaaaaataaaataggatatgaaaatgg

SEQ ID NO: 35 - protein, 69 aa, Homo sapiens [sequence omitted]

SEQ ID NO: 36 - DNA, 1330 bp, Homo sapiens
  source 1..1330: mol_type = other DNA
actcctctccgccagaccgccgccgcgccgccatcatggacaccagccgtgtgcagcctatcaagctggccagggtcaccaaggtcctgggcaggaccggttctcagggacagtgcacgcaggtgcgcgtggaattcatggacgacacgagccgatccatcatccgcaatgtaaaaggccccgtgcgcgagggcgacgtgctcacccttttggagtcagagcgagaagcccggaggttgcgctgagcttggctgctcgctgggtcttggatgtcgggttcgaccacttggccgatgggaatggtctgtcacagtctgctccttttttttgtccgccacacgtaactgagatgctcctttaaataaagcgtttgtgtttcaagttaactcaggttcttgtctgggttatacgactagggtttctccaggtttcttgagtggctcccaggcggtcaccgatcctccgcactctggaaatcctggccgtgcggtcttgccaaacgaagcttttcctttttgaggcggggggtcgtgtttgtcgattgcaccctctaccccaaacaaaacacaagcgtagtaggaatgttttattagcaaagaagtttcagagagtgggtggatcagggctctatcacttggtccccacctcaccttggtggggccagagtgagccccttcctgccacagtcaccccaactgaaattgcctttctcttcggccagtgttagcctctgagcaggggaccctggacccttctgtgcgccaaaggctgaggtgactgacgaggagatctccccacagctaggtgtagtgagccagacgaggcagcttactgaacctgggggttctctccattgtcaccgcattctccttcaccaggtgtggctgtctgggagccagggggtgactcgctctggagagaggggaaaagaggggggcctgctgcaatctccttgaggcaggaaacgtgggattcagccccagcctcacttagtggaggttcttttaccatggacccaggctgcctggtttgtatccaacctctgccccttctgacctggaagaggcgcttgaccttcctcccacatcccttccagtggggtgagtacaggtgttcctcagtttacaatgggttacattccggtgagtacatcataggttgaaagtattgcaagttgaaatgtgtttaatacacctaatctcccaaacatcacagcttagcatggtccatcttaagcttgttcagaacgccttagcctgtagttggggaaactcgtctaacacgaagcctgttttaataaagtattgaatgtcttatgtaatttattgaa

SEQ ID NO: 37 - protein, 204 aa, Homo sapiens [sequence omitted]

SEQ ID NO: 38 - DNA, 741 bp, Homo sapiens
  source 1..741: mol_type = other DNA
ctcttcctgtctgtaccagggcggcgcgtggtctacgccgagtgacagagacgctcaggctgtgttctcaggatgaccgagtgggagacagcagcaccagcggtggcagagaccccagacatcaagctctttgggaagtggagcaccgatgatgtgcagatcaatgacatttccctgcaggattacattgcagtgaaggagaagtatgccaagtacctgcctcacagtgcagggcggtatgccgccaaacgcttccgcaaagctcagtgtcccattgtggagcgcctcactaactccatgatgatgcacggccgcaacaacggcaagaagctcatgactgtgcgcatcgtcaagcatgccttcgagatcatacacctgctcacaggcgagaaccctctgcaggtcctggtgaacgccatcatcaacagtggtccccgggaggactccacacgcattgggcgcgccgggactgtgagacgacaggctgtggatgtgtcccccctgcgccgtgtgaaccaggccatctggctgctgtgcacaggcgctcgtgaggctgccttccggaacattaagaccattgctgagtgcctggcagatgagctcatcaatgctgccaagggctcctcgaactcctatgccattaagaagaaggacgagctggagcgtgtggccaagtccaaccgctgattttcccagctgctgcccaataaacctgtctgccctttggggcagtcccagcca

SEQ ID NO: 39 - protein, 194 aa, Homo sapiens [sequence omitted]

SEQ ID NO: 40 - DNA, 732 bp, Homo sapiens
  source 1..732: mol_type = other DNA
ctcgcgctgtttccgcctcttgccttcggacgccggattttgacgtgctctcgcgagatttgggtctcttcctaagccggcgctcggcaagttctcccaggagaaagccatgttcagttcgagcgccaagatcgtgaagcccaatggcgagaagccggacgagttcgagtccggcatctcccaggctcttctggagctggagatgaactcggacctcaaggctcagctcagggagctgaatattacggcagctaaggaaattgaagttggtggtggtcggaaagctatcataatctttgttcccgttcctcaactgaaatctttccagaaaatccaagtccggctagtacgcgaattggagaaaaagttcagtgggaagcatgtcgtctttatcgctcagaggagaattctgcctaagccaactcgaaaaagccgtacaaaaaataagcaaaagcgtcccaggagccgtactctgacagctgtgcacgatgccatccttgaggacttggtcttcccaagcgaaattgtgggcaagagaatccgcgtcaaactagatggcagccggctcataaaggttcatttggacaaagcacagcagaacaatgtggaacacaaggttgaaactttttctggtgtctataagaagctcacgggcaaggatgttaattttgaattcccagagtttcaattgtaaacaaaaatgactaaataaaaagtatatattcacagtac

SEQ ID NO: 41 - protein, 295 aa, Homo sapiens [sequence omitted]

SEQ ID NO: 42 - DNA, 1140 bp, Homo sapiens
  source 1..1140: mol_type = other DNA
cttttccgtgctacctgcagaggggtccatacggcgttgttctggattcccgtcgtaacttaaagggaaattttcacaatgtccggagcccttgatgtcctgcaaatgaaggaggaggatgtccttaagttccttgcagcaggaacccacttaggtggcaccaatcttgacttccagatggaacagtacatctataaaaggaaaagtgatggcatctatatcataaatctcaagaggacctgggagaagcttctgctggcagctcgtgcaattgttgccattgaaaaccctgctgatgtcagtgttatatcctccaggaatactggccagagggctgtgctgaagtttgctgctgccactggagccactccaattgctggccgcttcactcctggaaccttcactaaccagatccaggcagccttccgggagccacggcttcttgtggttactgaccccagggctgaccaccagcctctcacggaggcatcttatgttaacctacctaccattgcgctgtgtaacacagattctcctctgcgctatgtggacattgccatcccatgcaacaacaagggagctcactcagtgggtttgatgtggtggatgctggctcgggaagttctgcgcatgcgtggcaccatttcccgtgaacacccatgggaggtcatgcctgatctgtacttctacagagatcctgaagagattgaaaaagaagagcaggctgctgctgagaaggcagtgaccaaggaggaatttcagggtgaatggactgctcccgctcctgagttcactgctactcagcctgaggttgcagactggtctgaaggtgtacaggtgccctctgtgcctattcagcaattccctactgaagactggagcgctcagcctgccacggaagactggtctgcagctcccactgctcaggccactgaatgggtaggagcaaccactgactggtcttaagctgttcttgcataggctcttaagcagcatggaaaaatggttgatggaaaataaacatcagtttctaaaagttgtcttcatttagtttgctttttactccagatcagaatacctgggattgcatatcaaagcataataataaatacatgtctcgacatgagttgtacttctaaa

SEQ ID NO: 43 - protein, 298 aa, Homo sapiens
MGPKDSAKCLHRGPQPSHWAAGDGPTQERCGPRSLGSPVLGLDTCRAWDHVDGQILGQLRPLTEEEEEEGAGATLSRGPAFPGMGSEELRLASFYDWPLTAEVPPELLAAAGFFHTGHQDKVRCFFCYGGLQSWKRGDDPWTEHAKWFPSCQFLLRSKGRDFVHSVQETHSQLLGSWDPWEEPEDAAPVAPSVPASGYPELPTPRREVQSESAQEPGGVSPAEAQRAWWVLEPPGARDVEAQLRRLQEERTCKVCLDRAVSIVFVPCGHLVCAECAPGLQLCPICRAPVRSRVRTFLS

SEQ ID NO: 44 - DNA, 1334 bp, Homo sapiens
  source 1..1334: mol_type = other DNA
acttccagaaagctgtgggccctgggatactcccctcccagggtgtctggtggcaggcctgtgcctatccctgctgtccccagggtgggccccgggggtcaggagctccagaagggccagctgggcatattctgagattggccatcagcccccatttctgctgcaaacctggtcagagccagtgttccctccatgggacctaaagacagtgccaagtgcctgcaccgtggaccacagccgagccactgggcagccggtgatggtcccacgcaggagcgctgtggaccccgctctctgggcagccctgtcctaggcctggacacctgcagagcctgggaccacgtggatgggcagatcctgggccagctgcggcccctgacagaggaggaagaggaggagggcgccggggccaccttgtccagggggcctgccttccccggcatgggctctgaggagttgcgtctggcctccttctatgactggccgctgactgctgaggtgccacccgagctgctggctgctgccggcttcttccacacaggccatcaggacaaggtgaggtgcttcttctgctatgggggcctgcagagctggaagcgcggggacgacccctggacggagcatgccaagtggttccccagctgtcagttcctgctccggtcaaaaggaagagactttgtccacagtgtgcaggagactcactcccagctgctgggctcctgggacccgtgggaagaaccggaagacgcagcccctgtggccccctccgtccctgcctctgggtaccctgagctgcccacacccaggagagaggtccagtctgaaagtgcccaggagccaggaggggtcagtccagccgaggcccagagggcgtggtgggttcttgagcccccaggagccagggatgtggaggcgcagctgcggcggctgcaggaggagaggacgtgcaaggtgtgcctggaccgcgccgtgtccatcgtctttgtgccgtgcggccacctggtctgtgctgagtgtgcccccggcctgcagctgtgccccatctgcagagcccccgtccgcagccgcgtgcgcaccttcctgtcctaggccaggtgccatggccggccaggtgggctgcagagtgggctccctgcccctctctgcctgttctggactgtgttctgggcctgctgaggatggcagagctggtgtccatccagcactgaccagccctgattccccgaccaccgcccagggtggagaaggaggcccttgcttggcgtgggggatggcttaactgtacctgtttggatgcttctgaatagaaataaagtgggttttccctggaggta

SEQ ID NO: 45 - protein, 829 aa, Homo sapiens
MGLPRGPLASLLLLQVCWLQCAASEPCRAVFREAEVTLEAGGAEQEPGQALGKVFMGCPGQEPALFSTDNDDFTVRNGETVQERRSLKERNPLKIFPSKRILRRHKRDWVVAPISVPENGKGPFPQRLNQLKSNKDRDTKIFYSITGPGADSPPEGVFAVEKETGWLLLNKPLDREEIAKYELFGHAVSENGASVEDPMNISIIVTDQNDHKPKFTQDTFRGSVLEGVLPGTSVMQVTATDEDDAIYTYNGVVAYSIHSQEPKDPHDLMFTIHRSTGTISVISSGLDREKVPEYTLTIQATDMDGDGSTTTAVAVVEILDANDNAPMFDPQKYEAHVPENAVGHEVQRLTVTDLDAPNSPAWRATYLIMGGDDGDHFTITTHPESNQGILTTRKGLDFEAKNQHTLYVEVTNEAPFVLKLPTSTATIVVHVEDVNEAPVFVPPSKVVEVQEGIPTGEPVCVYTAEDPDKENQKISYRILRDPAGWLAMDPDSGQVTAVGTLDREDEQFVRNNIYEVMVLAMDNGSPPTTGTGTLLLTLIDVNDHGPVPEPRQITICNQSPVRQVLNITDKDLSPHTSPFQAQLTDDSDIYWTAEVNEEGDTVVLSLKKFLKQDTYDVHLSLSDHGNKEQLTVIRATVCDCHGHVETCPGPWKGGFILPVLGAVLALLFLLLVLLLLVRKKRKIKEPLLLPEDDTRDNVFYYGEEGGGEEDQDYDITQLHRGLEARPEVVLRNDVAPTIIPTPMYRPRPANPDEIGNFIIENLKAANTDPTAPPYDTLLVFDYEGSGSDAASLSSLTSSASDQDQDYDYLNEWGSRFKKLADMYGGGEDD

SEQ ID NO: 46 - DNA, 4452 bp, Homo sapiens
  source 1..4452: mol_type = other DNA
aaaggggcaagagctgagcggaacaccggcccgccgtcgcggcagctgcttcacccctctctctgcagccatggggctccctcgtggacctctcgcgtctctcctccttctccaggtttgctggctgcagtgcgcggcctccgagccgtgccgggcggtcttcagggaggctgaagtgaccttggaggcgggaggcgcggagcaggagcccggccaggcgctggggaaagtattcatgggctgccctgggcaagagccagctctgtttagcactgataatgatgacttcactgtgcggaatggcgagacagtccaggaaagaaggtcactgaaggaaaggaatccattgaagatcttcccatccaaacgtatcttacgaagacacaagagagattgggtggttgctccaatatctgtccctgaaaatggcaagggtcccttcccccagagactgaatcagctcaagtctaataaagatagagacaccaagattttctacagcatcacggggccgggggcagacagcccccctgagggtgtcttcgctgtagagaaggagacaggctggttgttgttgaataagccactggaccgggaggagattgccaagtatgagctctttggccacgctgtgtcagagaatggtgcctcagtggaggaccccatgaacatctccatcatcgtgaccgaccagaatgaccacaagcccaagtttacccaggacaccttccgagggagtgtcttagagggagtcctaccaggtacttctgtgatgcaggtgacagccacggatgaggatgatgccatctacacctacaatggggtggttgcttactccatccatagccaagaaccaaaggacccacacgacctcatgttcaccattcaccggagcacaggcaccatcagcgtcatctccagtggcctggaccgggaaaaagtccctgagtacacactgaccatccaggccacagacatggatggggacggctccaccaccacggcagtggcagtagtggagatccttgatgccaatgacaatgctcccatgtttgacccccagaagtacgaggcccatgtgcctgagaatgcagtgggccatgaggtgcagaggctgacggtcactgatctggacgcccccaactcaccagcgtggcgtgccacctaccttatcatgggcggtgacgacggggaccattttaccatcaccacccaccctgagagcaaccagggcatcctgacaaccaggaagggtttggattttgaggccaaaaaccagcacaccctgtacgttgaagtgaccaacgaggccccttttgtgctgaagctcccaacctccacagccaccatagtggtccacgtggaggatgtgaatgaggcacctgtgtttgtcccaccctccaaagtcgttgaggtccaggagggcatccccactggggagcctgtgtgtgtctacactgcagaagaccctgacaaggagaatcaaaagatcagctaccgcatcctgagagacccagcagggtggctagccatggacccagacagtgggcaggtcacagctgtgggcaccctcgaccgtgaggatgagcagtttgtgaggaacaacatctatgaagtcatggtcttggccatggacaatggaagccctcccaccactggcacgggaacccttctgctaacactgattgatgtcaatgaccatggcccagtccctgagccccgtcagatcaccatctgcaaccaaagccctgtgcgccaggtgctgaacatcacggacaaggacctgtctccccacacctcccctttccaggcccagctcacagatgactcagacatctactggacggcagaggtcaacgaggaaggtgacacagtggtcttgtccctgaagaagttcctgaagcaggatacatatgacgtgcacctttctctgtctgaccatggcaacaaagagcagctgacggtgatcagggccactgtgtgcgactgccatggccatgtcgaaacctgccctggaccctggaagggaggtttcatcctccctgtgctgggggctgtcctggctctgctgttcctcctgctggtgctgcttttgttggtgagaaagaagcggaagatcaaggagcccctcctactcccagaagatgacacccgtgacaacgtcttctactatggcgaagaggggggtggcgaagaggaccaggactatgacatcacccagctccaccgaggtctggaggccaggccggaggtggttctccgcaatgacgtggcaccaaccatcatcccgacacccatgtaccgtcctcggccagccaacccagatgaaatcggcaactttataattgagaacctgaaggcggctaacacagaccccacagccccgccctacgacaccctcttggtgttcgactatgagggcagcggctccgacgccgcgtccctgagctccctcacctcctccgcctccgaccaagaccaagattacgattatctgaacgagtggggcagccgcttcaagaagctggcagacatgtacggtggcggggaggacgactaggcggcctgcctgcagggctggggaccaaacgtcaggccacagagcatctccaaggggtctcagttcccccttcagctgaggacttcggagcttgtcaggaagtggccgtagcaacttggcggagacaggctatgagtctgacgttagagtggtggcttccttagcctttcaggatggaggaatgtgggcagtttgacttcagcactgaaaacctctccacctgggccagggttgcctcagaggccaagtttccagaagcctcttacctgccgtaaaatgctcaaccctgtgtcctgggcctgggcctgctgtgactgacctacagtggactttctctctggaatggaaccttcttaggcctcctggtgcaacttaatttttttttttaatgctatcttcaaaacgttagagaaagttcttcaaaagtgcagcccagagctgctgggcccactggccgtcctgcatttctggtttccagaccccaatgcctcccattcggatggatctctgcgtttttatactgagtgtgcctaggttgccccttattttttattttccctgttgcgttgctatagatgaagggtgaggacaatcgtgtatatgtactagaacttttttattaaagaaacttttcccagaggtgcctggggagtgaactgttttctaaatagaaggtttattggcatctaactcacataccataccattcacttgtttaacgtttacaattcaatggtttttagaattttcagagttctgcaaaaagagtgggctctgtttaccctgctggcttcacccaagcttcctctcaatggcaggggatactcagggtcagcttccatgcctaagtgggctcagggaggggagacttgccctcctttgcaaagtcagttgctccatgagagagaaccattaacctcttaccccaaggctgagcccctccacagccccagcaaggtctcttctggaacagcggctgccctccctgcaggacagtgtgtgacgacttcctcttctatgctggaggggtccctgttgaaaggcaggggttggggaacagccagctctgctacttgctagcacatttttttttttttttttgagacggagtctcgttctgtcgcccaggctggagtgcagtggtgtgatcttggctcactgcaagctctgcctcctgggttcacgccattctcctgcctcagcctcccgagtagctgggactacaggtgcctgccactatgcccagctaattttttttgagtgtgtgtgtttttttagtagagacggggtttcactgtgttagccaggatgtctcgatctcctgacctcgtgatccgcccacgtcggcctcccaaagtgctaggattacaggcttgagccaccgcgcccagcgctggcacatcatttaacctccagttgcctcagattttacatttacaaaatggggagtttttgtggagattaagtgaattaatatctggcacatggtccatgcaatctgttagttggtaacagctaccatttattcagtacttttaaaggccagacaggacttcgattatttcctctaaatcctcacaatcaccctctgaggggactttctcctttaaagaatggccacattgtatttgtttttttaaatgacatctggtcatcatcgaaatcaagcaaaacaaaattaagagaacctacccaagatgtcagtgaaattggaacattcctgacaataccagggcataaatgcaggaatcaggaataggcagcagtgatagaacaattctgtttgtgcccttgttaacgtgaagttcaaagtcatctttgcaattagccaaaagaatctgaagtgaagctgaggaaattgctgatgttgaaataaacatttccttccatga

SEQ ID NO: 47 - protein, 484 aa, Homo sapiens
MRHSKRTYCPDWDDKDWDYGKWRSSSSHKRRKRSHSSAQENKRCKYNHSKMCDSHYLESRSINEKDYHSRRYIDEYRNDYTQGCEPGHRQRDHESRYQNHSSKSSGRSGRSSYKSKHRIHHSTSHRRSHGKSHRRKRTRSVEDDEEGHLICQSGDVLSARYEIVDTLGEGAFGKVVECIDHKAGGRHVAVKIVKNVDRYCEAARSEIQVLEHLNTTDPNSTFRCVQMLEWFEHHGHICIVFELLGLSTYDFIKENGFLPFRLDHIRKMAYQICKSVNFLHSNKLTHTDLKPENILFVQSDYTEAYNPKIKRDERTLINPDIKVVDFGSATYDDEHHSTLVSTRHYRAPEVILALGWSQPCDVWSIGCILIEYYLGFTVFPTHDSKEHLAMMERILGPLPKHMIQKTRKRKYFHHDRLDWDEHSSAGRYVSRRCKPLKEFMLSQDVEHERLFDLIQKMLEYDPAKRITLREALKHPFFDLLKKSI

SEQ ID NO: 48 - DNA, 1847 bp, Homo sapiens
  source 1..1847: mol_type = other DNA
attttgttgttggtgcgcgacgcagtcagctgcgtgattcccgtgattgcgttacaagctttgtctccttcgacttggagtctttgtccaggacgatgagacactcaaagagaacttactgtcctgattgggatgacaaggattgggattatggaaaatggaggagcagcagcagtcataaaagaaggaagagatcacatagcagtgcccaggagaacaagcgctgcaaatacaatcactctaaaatgtgtgatagccattatttggaaagcaggtctataaatgagaaagattatcatagtcgacgctacattgatgagtacagaaatgactacactcaaggatgtgaacctggacatcgccaaagagaccatgaaagccggtatcagaaccatagtagcaagtcttctggtagaagtggaagaagtagttataaaagcaaacacaggattcaccacagtacttcacatcgtcgttcacatgggaagagtcaccgaaggaaaagaaccaggagtgtagaggatgatgaggagggtcacctgatctgtcagagtggagacgtactaagtgcaagatatgaaattgttgatactttaggtgaaggagcttttggaaaagttgtggagtgcatcgatcataaagcgggaggtagacatgtagcagtaaaaatagttaaaaatgtggatagatactgtgaagctgctcgctcagaaatacaagttctggaacatctgaatacaacagaccccaacagtactttccgctgtgtccagatgttggaatggtttgagcatcatggtcacatttgcattgtttttgaactattgggacttagtacttacgacttcattaaagaaaatggttttctaccatttcgactggatcatatcagaaagatggcatatcagatatgcaagtctgtgaattttttgcacagtaataagttgactcacacagacttaaagcctgaaaacatcttatttgtgcagtctgactacacagaggcgtataatcccaaaataaaacgtgatgaacgcaccttaataaatccagatattaaagttgtagactttggtagtgcaacatatgatgacgaacatcacagtacattggtatctacaagacattatagagcacctgaagttattttagccctagggtggtcccaaccatgtgatgtctggagcataggatgcattcttattgaatactatcttgggtttaccgtatttccaacacacgatagtaaggagcatttagcaatgatggaaaggattcttggacctctaccaaaacatatgatacagaaaaccaggaaacgtaaatattttcaccacgatcgattagactgggatgaacacagttctgccggcagatatgtttcaagacgctgtaaacctctgaaggaatttatgctttctcaagatgttgaacatgagcgtctctttgacctcattcagaaaatgttggagtatgatccagccaaaagaattactctcagagaagccttaaagcatcctttctttgaccttctgaagaaaagtatatagatctgtaattggacagctctctcgaagagatcttacagactgtatcagtctaatttttaaattttaagttattttgtacagctttgtaaattcttaacatttttatattgccatgtttattttgtttgggtaatttggttcattaagtacatagctaaggtaatgaacatctttttcagtaattgtaaagtgatttattcagaataaattttttgtgcttatgaagttgatatgtatctgaacagtttgttctaagtaccatttttcttcctacttctattaaagaatggacataga

SEQ ID NO: 49 - protein, 2322 aa, Homo sapiens
MQSGPRPPLPAPGLALALTLTMLARLASAASFFGENHLEVPVATALTDIDLQLQFSTSQPEALLLLAAGPADHLLLQLYSGRLQVRLVLGQEELRLQTPAETLLSDSIPHTVVLTVVEGWATLSVDGFLNASSAVPGAPLEVPYGLFVGGTGTLGLPYLRGTSRPLRGCLHAATLNGRSLLRPLTPDVHEGCAEEFSASDDVALGFSGPHSLAAFPAWGTQDEGTLEFTLTTQSRQAPLAFQAGGRRGDFIYVDIFEGHLRAVVEKGQGTVLLHNSVPVADGQPHEVSVHINAHRLEISVDQYPTHTSNRGVLSYLEPRGSLLLGGLDAEASRHLQEHRLGLTPEATNASLLGCMEDLSVNGQRRGLREALLTRNMAAGCRLEEEEYEDDAYGHYEAFSTLAPEAWPAMELPEPCVPEPGLPPVFANFTQLLTISPLVVAEGGTAWLEWRHVQPTLDLMEAELRKSQVLFSVTRGARHGELELDIPGAQARKMFTLLDVVNRKARFIHDGSEDTSDQLVLEVSVTARVPMPSCLRRGQTYLLPIQVNPVNDPPHIIFPHGSLMVILEHTQKPLGPEVFQAYDPDSACEGLTFQVLGTSSGLPVERRDQPGEPATEFSCRELEAGSLVYVHRGGPAQDLTFRVSDGLQASPPATLKVVAIRPAIQIHRSTGLRLAQGSAMPILPANLSVETNAVGQDVSVLFRVTGALQFGELQKQGAGGVEGAEWWATQAFHQRDVEQGRVRYLSTDPQHHAYDTVENLALEVQVGQEILSNLSFPVTIQRATVWMLRLEPLHTQNTQQETLTTAHLEATLEEAGPSPPTFHYEVVQAPRKGNLQLQGTRLSDGQGFTQDDIQAGRVTYGATARASEAVEDTFRFRVTAPPYFSPLYTFPIHIGGDPDAPVLTNVLLVVPEGGEGVLSADHLFVKSLNSASYLYEVMERPRHGRLAWRGTQDKTTMVTSFTNEDLLRGRLVYQHDDSETTEDDIPFVATRQGESSGDMAWEEVRGVFRVAIQPVNDHAPVQTISRIFHVARGGRRLLTTDDVAFSDADSGFADAQLVLTRKDLLFGSIVAVDEPTRPIYRFTQEDLRKRRVLFVHSGADRGWIQLQVSDGQHQATALLEVQASEPYLRVANGSSLVVPQGGQGTIDTAVLHLDTNLDIRSGDEVHYHVTAGPRWGQLVRAGQPATAFSQQDLLDGAVLYSHNGSLSPRDTMAFSVEAGPVHTDATLQVTIALEGPLAPLKLVRHKKIYVFQGEAAEIRRDQLEAAQEAVPPADIVFSVKSPPSAGYLVMVSRGALADEPPSLDPVQSFSQEAVDTGRVLYLHSRPEAWSDAFSLDVASGLGAPLEGVLVELEVLPAAIPLEAQNFSVPEGGSLTLAPPLLRVSGPYFPTLLGLSLQVLEPPQHGALQKEDGPQARTLSAFSWRMVEEQLIRYVHDGSETLTDSFVLMANASEMDRQSHPVAFTVTVLPVNDQPPILTTNTGLQMWEGATAPIPAEALRSTDGDSGSEDLVYTIEQPSNGRVVLRGAPGTEVRSFTQAQLDGGLVLFSHRGTLDGGFRFRLSDGEHTSPGHFFRVTAQKQVLLSLKGSQTLTVCPGSVQPLSSQTLRASSSAGTDPQLLLYRVVRGPQLGRLFHAQQDSTGEALVNFTQAEVYAGNILYEHEMPPEPFWEAHDTLELQLSSPPARDVAATLAVAVSFEAACPQRPSHLWKNKGLWVPEGQRARITVAALDASNLLASVPSPQRSEHDVLFQVTQFPSRGQLLVSEEPLHAGQPHFLQSQLAAGQLVYAHGGGGTQQDGFHFRAHLQGPAGASVAGPQTSEAFAITVRDVNERPPQPQASVPLRLTRGSRAPISRAQLSVVDPDSAPGEIEYEVQRAPHNGFLSLVGGGLGPVTRFTQADVDSGRLAFVANGSSVAGIFQLSMSDGASPPLPMSLAVDILPSAIEVQLRAPLEVPQALGRSSLSQQQLRVVSDREEPEAAYRLIQGPQYGHLLVGGRPTSAFSQFQIDQGEVVFAFTNFSSSHDHFRVLALARGVNASAVVNVTVRALLHVWAGGPWPQGATLRLDPTVLDAGELANRTGSVPRFRLLEGPRHGRVVRVPRARTEPGGSQLVEQFTQQDLEDGRLGLEVGRPEGRAPGPAGDSLTLELWAQGVPPAVASLDFATEPYNAARPYSVALLSVPEAARTEAGKPESSTPTGEPGPMASSPEPAVAKGGFLSFLEANMFSVIIPMCLVLLLLALILPLLFYLRKRNKTGKHDVQVLTAKPRNGLAGDTETFRKVEPGQAIPLTAVPGQGPPPGGQPDPELLQFCRTPNPALKNGQYWV

SEQ ID NO: 50 - DNA, 8290 bp, Homo sapiens
  source 1..8290: mol_type = other DNA
gcgcccaggagcagagccgcgctcgctccactcagctcccagctcccaggactccgctggctcctcgcaagtcctgccgcccagcccgccgggatgcagtccgggccgcggcccccacttccagcccccggcctggccttggctttgaccctgactatgttggccagacttgcatccgcggcttccttcttcggtgagaaccacctggaggtgcctgtggccacggctctgaccgacatagacctgcagctgcagttctccacgtcccagcccgaagccctccttctcctggcagcaggcccagctgaccacctcctgctgcagctctactctggacgcctgcaggtcagacttgttctgggccaggaggagctgaggctgcagactccagcagagacgctgctgagtgactccatcccccacactgtggtgctgactgtcgtagagggctgggccacgttgtcagtcgatgggtttctgaacgcctcctcagcagtcccaggagcccccctagaggtcccctatgggctctttgttgggggcactgggacccttggcctgccctacctgaggggaaccagccgacccctgaggggttgcctccatgcagccaccctcaatggccgcagcctcctccggcctctgacccccgatgtgcatgagggctgtgctgaagagttttctgccagtgatgatgtggccctgggcttctctgggccccactctctggctgccttccctgcctggggcactcaggacgaaggaaccctagagtttacactcaccacacagagccggcaggcacccttggccttccaggcagggggccggcgtggggacttcatctatgtggacatatttgagggccacctgcgggccgtggtggagaagggccagggtaccgtattgctccacaacagtgtgcctgtggccgatgggcagccccatgaggtcagtgtccacatcaatgctcaccggctggaaatctccgtggaccagtaccctacgcatacttcgaaccgaggagtcctcagctacctggagccacggggcagtctccttctcggggggctggatgcagaggcctctcgtcacctccaggaacaccgcctgggcctgacaccagaggccaccaatgcctccctgctgggctgcatggaagacctcagtgtcaatggccagaggcgggggctgcgggaagctttgctgacgcgcaacatggcagccggctgcaggctggaggaggaggagtatgaggacgatgcctatggacattatgaagctttctccaccctggcccctgaggcttggccagccatggagctgcctgagccatgcgtgcctgagccagggctgcctcctgtctttgccaatttcacccagctgctgactatcagcccactggtggtggccgaggggggcacagcctggcttgagtggaggcatgtgcagcccacgctggacctgatggaggctgagctgcgcaaatcccaggtgctgttcagcgtgacccgaggggcacgccatggcgagctcgagctggacatcccgggagcccaggcacgaaaaatgttcaccctcctggacgtggtgaaccgcaaggcccgcttcatccacgatggctctgaggacacctccgaccagctggtgctggaggtgtcggtgacggctcgggtgcccatgccctcatgccttcggaggggccaaacatacctcctgcccatccaggtcaaccctgtcaatgacccaccccacatcatcttcccacatggcagcctcatggtgatcctggaacacacgcagaagccgctggggcctgaggttttccaggcctatgacccggactctgcctgtgagggcctcaccttccaggtccttggcacctcctctggcctccccgtggagcgccgagaccagcctggggagccggcgaccgagttctcctgccgggagttggaggccggcagcctagtctatgtccaccgcggtggtcctgcacaggacttgacgttccgggtcagcgatggactgcaggccagccccccggccacgctgaaggtggtggccatccggccggccatacagatccaccgcagcacagggttgcgactggcccaaggctctgccatgcccatcttgcccgccaacctgtcggtggagaccaatgccgtggggcaggatgtgagcgtgctgttccgcgtcactggggccctgcagtttggggagctgcagaagcagggggcaggtggggtggagggtgctgagtggtgggccacacaggcgttccaccagcgggatgtggagcagggccgcgtgaggtacctgagcactgacccacagcaccacgcttacgacaccgtggagaacctggccctggaggtgcaggtgggccaggagatcctgagcaatctgtccttcccagtgaccatccagagagccactgtgtggatgctgcggctggagccactgcacactcagaacacccagcaggagaccctcaccacagcccacctggaggccaccctggaggaggcaggcccaagccccccaaccttccattatgaggtggttcaggctcccaggaaaggcaaccttcaactacagggcacaaggctgtcagatggccagggcttcacccaggatgacatacaggctggccgggtgacctatggggccacagcacgtgcctcagaggcagtcgaggacaccttccgtttccgtgtcacagctccaccatatttctccccactctataccttccccatccacattggtggtgacccagatgcgcctgtcctcaccaatgtcctcctcgtggtgcctgagggtggtgagggtgtcctctctgctgaccacctctttgtcaagagtctcaacagtgccagctacctctatgaggtcatggagcggccccgccatgggaggttggcttggcgtgggacacaggacaagaccactatggtgacatccttcaccaatgaagacctgttgcgtggccggctggtctaccagcatgatgactccgagaccacagaagatgatatcccatttgttgctacccgccagggcgagagcagtggtgacatggcctgggaggaggtacggggtgtcttccgagtggccatccagcccgtgaatgaccacgcccctgtgcagaccatcagccggatcttccatgtggcccggggtgggcggcggctgctgactacagacgacgtggccttcagcgatgctgactcgggctttgctgacgcccagctggtgcttacccgcaaggacctcctctttggcagtatcgtggccgtagatgagcccacgcggcccatctaccgcttcacccaggaggacctcaggaagaggcgagtactgttcgtgcactcaggggctgaccgtggctggatccagctgcaggtgtccgacgggcaacaccaggccactgcgctgctggaggtgcaggcctcggaaccctacctccgtgtggccaacggctccagccttgtggtccctcaaggaggccagggcaccatcgacacggccgtgctccacctggacaccaacctcgacatccgcagtggggatgaggtccactaccacgtcacagctggccctcgctggggacagctagtccgggctggtcagccagccacagccttctcccagcaggacctgctggatggggccgttctctatagccacaatggcagcctcagcccccgcgacaccatggccttctccgtggaagcagggccagtgcacacggatgccaccctacaagtgaccattgccctagagggcccactggccccactgaagctggtccggcacaagaagatctacgtcttccagggagaggcagctgagatcagaagggaccagctggaggcagcccaggaggcagtgccacctgcagacatcgtattctcagtgaagagcccaccgagtgccggctacctggtgatggtgtcgcgtggcgccttggcagatgagccacccagcctggaccctgtgcagagcttctcccaggaggcagtggacacaggcagggtcctgtacctgcactcccgccctgaggcctggagcgatgccttctcgctggatgtggcctcaggcctgggtgctcccctcgagggcgtccttgtggagctggaggtgctgcccgctgccatcccactagaggcgcaaaacttcagcgtccctgagggtggcagcctcaccctggcccctccactgctccgtgtctccgggccctacttccccactctcctgggcctcagcctgcaggtgctggagccaccccagcatggagccctgcagaaggaggacggacctcaagccaggaccctcagcgccttctcctggagaatggtggaagagcagctgatccgctacgtgcatgacgggagcgagacactgacagacagttttgtcctgatggctaatgcctccgagatggatcgccagagccatcctgtggccttcactgtcactgtcctgcctgtcaatgaccaaccccccatcctcactacaaacacaggcctgcagatgtgggagggggccactgcgcccatccctgcggaggctctgaggagcacggacggcgactctgggtctgaggatctggtctacaccatcgagcagcccagcaacgggcgggtagtgctgcggggggcgccgggcactgaggtgcgcagcttcacgcaggcccagctggacggcgggctcgtgctgttctcacacagaggaaccctggatggaggcttccgcttccgcctctctgacggcgagcacacttcccccggacacttcttccgagtgacggcccagaagcaagtgctcctctcgctgaagggcagccagacactgactgtctgcccagggtccgtccagccactcagcagtcagaccctcagggccagctccagcgcaggcactgacccccagctcctgctctaccgtgtggtgcggggcccccagctaggccggctgttccacgcccagcaggacagcacaggggaggccctggtgaacttcactcaggcagaggtctacgctgggaatattctgtatgagcatgagatgccccccgagcccttttgggaggcccatgataccctagagctccagctgtcctcgccgcctgcccgggacgtggccgccacccttgctgtggctgtgtcttttgaggctgcctgtccccagcgccccagccacctctggaagaacaaaggtctctgggtccccgagggccagcgggccaggatcaccgtggctgctctggatgcctccaatctcttggccagcgttccatcaccccagcgctcagagcatgatgtgctcttccaggtcacacagttccccagccggggccagctgttggtgtccgaggagcccctccatgctgggcagccccacttcctgcagtcccagctggctgcagggcagctagtgtatgcccacggcggtgggggcacccagcaggatggcttccactttcgtgcccacctccaggggccagcaggggcctccgtggctggaccccaaacctcagaggcctttgccatcacggtgagggatgtaaatgagcggccccctcagccacaggcctctgtcccactccggctcacccgaggctctcgtgcccccatctcccgggcccagctgagtgtggtggacccagactcagctcctggggagattgagtacgaggtccagcgggcaccccacaacggcttcctcagcctggtgggtggtggcctggggcccgtgacccgcttcacgcaagccgatgtggattcagggcggctggccttcgtggccaacgggagcagcgtggcaggcatcttccagctgagcatgtctgatggggccagcccacccctgcccatgtccctggctgtggacatcctaccatccgccatcgaggtgcagctgcgggcacccctggaggtgccccaagctttggggcgctcctcactgagccagcagcagctccgggtggtttcagatcgggaggagccagaggcagcataccgcctcatccagggaccccagtatgggcatctcctggtgggcgggcggcccacctcggccttcagccaattccagatagaccagggcgaggtggtctttgccttcaccaacttctcctcctctcatgaccacttcagagtcctggcactggctaggggtgtcaatgcatcagccgtagtgaacgtcactgtgagggctctgctgcatgtgtgggcaggtgggccatggccccagggtgccaccctgcgcctggaccccaccgtcctagatgctggcgagctggccaaccgcacaggcagtgtgccgcgcttccgcctcctggagggaccccggcatggccgcgtggtccgcgtgccccgagccaggacggagcccgggggcagccagctggtggagcagttcactcagcaggaccttgaggacgggaggctggggctggaggtgggcaggccagaggggagggcccccggccccgcaggtgacagtctcactctggagctgtgggcacagggcgtcccgcctgctgtggcctccctggactttgccactgagccttacaatgctgcccggccctacagcgtggccctgctcagtgtccccgaggccgcccggacggaagcagggaagccagagagcagcacccccacaggcgagccaggccccatggcatccagccctgagcccgctgtggccaagggaggcttcctgagcttccttgaggccaacatgttcagcgtcatcatccccatgtgcctggtacttctgctcctggcgctcatcctgcccctgctcttctacctccgaaaacgcaacaagacgggcaagcatgacgtccaggtcctgactgccaagccccgcaacggcctggctggtgacaccgagacctttcgcaaggtggagccaggccaggccatcccgctcacagctgtgcctggccaggggccccctccaggaggccagcctgacccagagctgctgcagttctgccggacacccaaccctgcccttaagaatggccagtactgggtgtgaggcctggcctgggcccagatgctgatcgggccagggacaggcttgcccatgtcccgggccccattgcttccatgcctggtgctgtctgagtatccccagagcaagagagacctggagacaccaggggtggagggtcctgggagatagtcccaggggtccgggacagagtggagtcaagagctggaacctccctcagctcactccgagcctggagaactgcaggggccaaggtggaggcaggcttaagttcagtcctcctgccctggagctggtttgggctgtcaaaaccagggtaacctcctacatgggtcatgactctgggtcctgggtctgtgaccttgggtaagtcgcgcctgacccaggctgctaagagggcaaggagaaggaagtaccctggggagggaagggacagaggaagctattcctggcttttccactccaacccaggccaccctttgtctctgccccagagttgagaaaaaaacttcctcccctggttttttagggagatggtatcccctggagtagagggcaagaggagagagcgcctccagtctagaaggcataagccaataggataatatattcagggtgcagggtgggtaggttgctctggggatgggtttatttaagggagattgcaaggaagctatttaacatggtgctgagctagccaggactgatggagcccctgggggtgtgggatggaggagggtctgcagccagttcattcccagggccccatcttgatgggccaagggctaaacatgcatgtgtcagtggctttggagcaggttaggctggggctcatcgagggtctcaggccgaggccactgcggtgccagtgcccccctgaggactagggcaggcagctgggggcacttggttccatggagcctggataaacagtgctttggaggctctggacagctgtgtggtgtttgtgtcttaactatgcactgggcccttgtctgcgtcggcttgcatacagagggcccctggggtcggccctccggcctggcctcagccagtgggatggacagggccaggcaggcctctgaacttccacctcctggggcctcccagacctcctgtgcccccacctgtgtgggcaggtgggccagtcttcgggtgatgggaccaaaccccttcagttcagtagagaaaggctaggtcctctacaaagagctgcaagacaaaaattaaaataaatgctccccaccctaga

SEQ ID NO: 51 - protein, 616 aa, Homo sapiens
MAASAKKKNKKGKTISLTDFLAEDGGTGGGSTYVSKPVSWADETDDLEGDVSTTWHSNDDDVYRAPPIDRSILPTAPRAAREPNIDRSRLPKSPPYTAFLGNLPYDVTEESIKEFFRGLNISAVRLPREPSNPERLKGFGYAEFEDLDSLLSALSLNEESLGNRRIRVDVADQAQDKDRDDRSFGRDRNRDSDKTDTDWRARPATDSFDDYPPRRGDDSFGDKYRDRYDSDRYRDGYRDGYRDGPRRDMDRYGGRDRYDDRGSRDYDRGYDSRIGSGRRAFGSGYRRDDDYRGGGDRYEDRYDRRDDRSWSSRDDYSRDDYRRDDRGPPQRPKLNLKPRSTPKEDDSSASTSQSTRAASIFGGAKPVDTAAREREVEERLQKEQEKLQRQLDEPKLERRPRERHPSWRSEETQERERSRTGSESSQTGTSTTSSRSKSDQDARRRESEKSLENETLNKEEDCHSPTSKPPKPDQPLKVMPAPPPKENAWVKRSSNPPARSQSSDTEQQSPTSGGGKVAPAQPSEEGPGRKDENKVDGMNAPKGQTGNSSRGPGDGGNRDHWKESDRKDGKKDQDSRSAPEPKKPEENPASKFSSASKYAALSVDGEDENEGEDYAE

SEQ ID NO: 52 - DNA, 3928 bp, Homo sapiens
  source 1..3928: mol_type = other DNA
cttttgcgttctctttccctctcccaacatggcggcctcagcaaaaaagaagaataagaaggggaagactatctccctaacagactttctggctgaggatgggggtactggtggaggaagcacctatgtttccaaaccagtcagctgggctgatgaaacggatgacctggaaggagatgtttcgaccacttggcacagtaacgatgacgatgtgtatagggcgcctccaattgaccgttccatccttcccactgctccacgggctgctcgggaacccaatatcgaccggagccgtcttcccaaatcgccaccctacactgcttttctaggaaacctaccctatgatgttacagaagagtcaattaaggaattctttcgaggattaaatatcagtgcagtgcgtttaccacgtgaacccagcaatccagagaggttgaaaggttttggttatgctgaatttgaggacctggattccctgctcagtgccctgagtctcaatgaagagtctctaggtaacaggagaattcgagtggacgttgctgatcaagcacaggataaagacagggatgatcgttcttttggccgtgatagaaatcgggattctgacaaaacagatacagactggagggctcgtcctgctacagacagctttgatgactacccacctagaagaggtgatgatagctttggagacaagtatcgagatcgttatgattcagaccggtatcgggatgggtatcgggatgggtatcgggatggcccacgccgggatatggatcgatatggtggccgggatcgctatgatgaccgaggcagcagagactatgatagaggctatgattcccggataggcagtggcagaagagcatttggcagtgggtatcgcagggatgatgactacagaggaggcggggaccgctatgaagaccgatatgacagacgggatgatcggtcgtggagctccagagatgattactctcgggatgattataggcgtgatgatagaggtcccccccaaagacccaaactgaatctaaagcctcggagtactcctaaggaagatgattcctctgctagtacctcccagtccactcgagctgcttctatctttggaggggcaaagcctgttgacacagctgctagagaaagagaagtagaagaacggctacagaaggaacaagagaagttgcagcgtcagctggatgagccaaaactagaacgacggcctcgggagagacacccaagctggcgaagtgaagaaactcaggaacgggaacggtcgaggacaggaagtgagtcatcacaaactgggacctccaccacatctagcagaagtaagtcagaccaggatgcacgaaggagagagagtgagaagtctctagaaaatgaaacactcaataaggaggaagattgccactctccaacttctaaacctcccaaacctgatcagcccctaaaggtaatgccagcccctccaccaaaggagaatgcttgggtgaagcgaagttctaaccctcctgctcgatctcagagctcagacacagagcagcagtcccctacaagtggtgggggaaaagtagctccagctcaaccatctgaggaaggaccaggaaggaaagatgaaaataaagtagatgggatgaatgccccaaaaggccaaactgggaactctagccgtggtccaggcgacggagggaacagagaccactggaaggagtcagataggaaagatggcaaaaaggatcaagactccagatctgcacctgagccaaagaaacctgaggaaaatccagcttccaagttcagttctgcaagcaagtatgctgctctctctgttgatggtgaagatgaaaatgagggagaagattatgccgaatagacctctacatcctgtgcttttctcctagtttctctccaccctggaacattcgagagcaaatcaaaacctctatccagacaagacaaaataaaactcaccatctcctgaagacctttcttacctttttttaaaaacaaaaaatgaaattattttgcatgctgctgcagcctttaaagtattgaagtaactggagaattgccaatacagccagagagaaagggactacagctttttagaggaaaagttgtggtgcgttatgtcaccatgcagttgccagtgtgattagtgcctaggggtctccatttagcagaaatggtaatgacagtgatataatgcctggaacctggttgggcagtaggggagggaggtagaaggaaaagtgtgagatttctaccttttagtttttatcctattgtggcatatatgaattctcaaacattatctgaataaattttccactcttggaaaggtagatttagcctcaagttgttctagtctccaggaggctgccagcccctcctcttatttaattctgagttttgggggccagcctagagggaattccttttttttttttaaccccccaggggggtagttgggagtgagactataggccataaagaatgggactgcattggaccaaaataaatgggaaaatcgtggtttgaaaagaagcttttgggaagtgatgagtcattttgcaccaggtaataggggaaaattgtgtgacctccagcaaacacatgaatggttatttcctggagccggaagcacttgggggtcgtggtaattcccagtgttttctgtgtcctagttttaccctttctaaacactgtcctttttgaaagttttgaatatatccacattctattgaaaccttgaaactaaaaatttagactcttatcatcatcttaagttcttcatgctactcttaacctcccaaaaagcagtatctaagtcacatacatgatgtcttgggcattttctcagccatggagaactctgaaaggaagaatcgctgcttttctcaagcaaatcggtttcttgatgtcttttggttctccttgcctgctcctgatgcttggaccccttttattgatcagagtgctctagaataatggatggtcttggatgatggataaatagggacagggacagttaaattgggagcctttcttacaaccttgatgggatttttccccccaagtttccttctccactgaaatgccacactaatgcttgttggattcatgaggtggccagaccaatgtgttgttttgttgttgtttttttaagcttcccttgagagaataaatggtaatggagagaactatttaacaaggtcctggtttctcttgcaacacagtagctaaacttgcctgcttttatatgcatttttgtagggatcagcttggtagacagtattagcggagaaacaccttgatcttggtttgcaagcccttctcccatcagtcctagattaggccctgttcagccatgcaggggtgttggtttatgcgtgctgcagcagtgggcataatgaatataatttacccagtggacaaaggtgtgtaccaagtgaatttaaataattggtgtggattggccagtagctaagaagtgggcttttaaagagtattgaagattgaaagggtttttctttcttttttaaaaaagaaaaacaaactattgattgtagataatgaaaagctagggtttgccctcttcatgtctactctccttccaaatagttatatccaaaactgtttttccctctcccctaccttgtcccccctattaaaatagaaacagggattgattaatgtcccgctcctgaatacatgtaaaatttgtacaaaaatatcttctatgaaaatgatttgtaatctgtagacttattacctgggagatgtcttgatgtaaaatcccatcctttgggttgtgggttttttgttttctccaaataaatctgatctttaaagttcattgtaaaatcgtcggtttctttctggacactgacttcaaaaataaaataggatatgaaaatgg

SEQ ID NO: 53 - protein, 127 aa, Homo sapiens
MRPLDIVELAEPEEVEVLEPEEDFEQFLLPVINEMREDIASLTREHGRAYLRNRSKLWEMDNMLIQIKTQVEASEESALNHLQNPGDAAEGRAAKRCEKAEEKAKEIAKMAEMLVELVRRIEKSESS

SEQ ID NO: 54 - DNA, 1574 bp, Homo sapiens
  source 1..1574: mol_type = other DNA
attttgttcgccgttactctgcgcgtaagtcgcttgtccgtggcttctctgagaagaaaagttgaaaaagggtaaaagttttcaggaatattcgggctctctattgctaagcatagcgagtgtcggttttctctctccaacagacatcgctattgcggttccgaggcagtgggaagagatgcggcccctggacatcgtcgagctggcggaaccggaggaagtggaggtgctggagcccgaggaggatttcgagcagtttctgctcccggtcatcaacgagatgcgcgaggacatcgcgtcgctgacgcgcgagcacgggcgggcgtacctgcggaaccggagcaagctgtgggagatggacaatatgctcatccagatcaaaacgcaggtggaggcctcggaggagagcgccctcaaccacctccagaacccgggcgacgcggccgagggccgggcggccaagaggtgcgagaaggccgaggagaaggccaaggagattgcgaagatggcagagatgctggtggagctggtccggcggatagagaagagcgagtcgtcgtgagcgcggtcggcggtttccagccaatggattctggtcaactggtggagattggctgacaccctggagaagccgaaaccagagagccttttgttttctcttttttcctgtctatgctctgtctcacttaacactacgttttctgctatggtctgtggttgatgacctcaatatgagtttcgattgttaacgtgtttttgtttgggaagtaattttgtttgaaaatgctctcacatacaggaattagggcctagattgtaagctcttgcagcagtcacatttgttcccgggctttggtggttatttctaaatttttgaggtgctttgctatttcttgtgtgacctgatagctccctggaactttgggtctgtgtgtgacacatgagactcacagttggagttctccagctctggaggtgctgaaggagctgcattaattctggaagacgactccatgcagcaactactgaagaaaggaccagacttcaacggggagtgtggatgggccgacctggctgggactcgtgaatctggagaagagctggagaatggatagtattgtctgtatttggagactttaatttctgtgtgagaccaaaggaggagagatgtgttttgttcaaaatttaaatttgttgtggtacactatcttatgtaacctgtctggtgagtttgtttggacaacctaactcagctttatttgacatggaacctaaaatagaagataagatcttgatattctgtacaagttgatgtaataccctgatgcgttttagaggacttggcataaaatgaaagattggcaaaggcccttgaggggcttggggatgacagtatggaactgtctgcattggaccctaaactggactagaagaggcatcttcaaggttcatacgttgtccagctgtaagttcatttgagtagcagacctaacaaatatttgaggtcagaaccctaccatgttaaaacaaacaaaaacttaccatgttaataaaagtattcatttgcttgaaaa

SEQ ID NO: 55 - protein, 479 aa, Homo sapiens
MSDGFDRAPGAGRGRSRGLGRGGGGPEGGGFPNGAGPAERARHQPPQPKAPGFLQPPPLRQPRTTPPPGAQCEVPASPQRPSRPGALPEQTRPLRAPPSSQDKIPQQNSESAMAKPQVVVAPVLMSKLSVNAPEFYPSGYSSSYTESYEDGCEDYPTLSEYVQDFLNHLTEQPGSFETEIEQFAETLNGCVTTDDALQELVELIYQQATSIPNFSYMGARLCNYLSHHLTISPQSGNFRQLLLQRCRTEYEVKDQAAKGDEVTRKRFHAFVLFLGELYLNLEIKGTNGQVTRADILQVGLRELLNALFSNPMDDNLICAVKLLKLTGSVLEDAWKEKGKMDMEEIIQRIENVVLDANCSRDVKQMLLKLVELRSSNWGRVHATSTYREATPENDPNYFMNEPTFYTSDGVPFTAADPDYQEKYQELLEREDFFPDYEENGTDLSGAGDPYLDDIDDEMDPEIEEAYEKFCLESERKRKQ

SEQ ID NO: 56 - DNA, 2780 bp, Homo sapiens
  source 1..2780: mol_type = other DNA
ggaaagccgagggtagccgagcggggcgggcgctctggagcggcgggtgctcgggctgccgtccgctccgccagaagcaccgagcagccgagccggggcccgccgccctcctcctccatgaggcccgagtgaggcgcggcggctatagccgacccgcggcgccttccccccgcgtcctatcgcgagcgcagcggcagcggcccctggaggaggaggcggaggaggaggagcatgtcggacggtttcgatcgggccccaggtgctggtcggggccggagccggggcctgggccgcggagggggcgggcctgagggcggcggtttcccgaacggagcggggcctgctgagcgggcgcggcaccagccgccgcaacccaaagccccgggcttcctgcagccaccgccgctgcgccagcccaggacgaccccgccgccaggggcccagtgcgaggtccccgccagcccccagcggccttcccggcccggggcgctcccagagcaaacgaggcccctgagagctccacctagttcacaggataaaatcccacagcagaactcggagtcagcaatggctaagccccaggtggttgtagctcctgtattaatgtctaagctgtctgtgaatgcccctgaattttacccttcaggttattcttccagttacacagaatcctatgaggatggttgtgaggattatcctactctatcagaatatgttcaggattttttgaatcatcttacagagcagcctggcagttttgaaactgaaattgaacagtttgcagagaccctgaatggttgtgttacaacagatgatgctttgcaagaacttgtggaactcatctatcaacaggccacatctatcccaaatttctcttatatgggagctcgcctgtgtaattacctgtcccatcatctgacaattagcccacagagtggcaacttccgccaattgctacttcaaagatgtcggactgaatatgaagttaaagatcaagctgcaaaaggggatgaagttactcgaaaacgatttcatgcatttgtactctttctgggagaactttatcttaacctggagatcaagggaacaaatggacaggttacaagagcagatattcttcaggttggtcttcgagaattgctgaatgccctgttttctaatcctatggatgacaatttaatttgtgcagtaaaattgttaaagttgacaggatcagttttggaagatgcttggaaggaaaaaggaaagatggatatggaagaaattattcagagaattgaaaacgttgtcctagatgcaaactgcagtagagatgtaaaacagatgctcttgaagcttgtagaactccggtcaagtaactggggcagagtccatgcaacttcaacatatagagaagcaacaccagaaaatgatcctaactactttatgaatgaaccaacattttatacatctgatggtgttcctttcactgcagctgatccagattaccaagagaaataccaagaattacttgaaagagaggacttttttccagattatgaagaaaatggaacagatttatccggggctggtgatccatacttggatgatattgatgatgagatggacccagagatagaagaagcttatgaaaagttttgtttggaatcagagcgtaagcgaaaacagtaaagttaaatttcagcatatcagttttataaagcagtttaggtatggtgatttagcagaacacaagagagcaagaaaatgtcacatctataccaaattaaggatgttgagttatgttactaatgtatgcaactttaattttgtttaacactatctgccaaaataaactttattccctataacttaaaatgtgtatatatatataatagtttattatgtacagttaattctactgttttggctgcaataaaatcgattttgaaataaatgaaatgttgaaaattttgctagttggttagatgcttatcctttaaattctacttttcttgaggggaaaaagtcttcttctggaaatacatattactgcaaaaatgtagcatccttttttaggtaggagtattatagctttcattttagtttgacatttagtgtcccaatgaattgaatttcaaatatgaatcataatcttgaaaatctttagcactaaagtcttggaatatatcaacaactgatttacatatgcagatgctatttgataccaagggctttttaaatgtcatgggggggaaaaacccaacttggtgaactcccagctaaacaaccaagacttcactgaagatttattccaattctagaattgttcttttttatttttattttttcaactgactaacttcattaccttaaaggctagaacattattctgctttatttatatggctttctcacttttattttgtagcatgggttgcatcgacttttttactagagaattttactagatatttgtcattcaagttttcatctgctttataattgatacaccttgagggtcacttttctaatacttttactataatgtggtaccacctcagccctaataaataatatttttacctaatgtcaaatctttttccagctaactaaaaactgtgtacaaaaggattgcttgtaaatatgcatgtaaatagttctgttaataacccactgttttacatttggtacatctgtgtctgctaatacagttagctttctcacttttctgcttgtttgttcagtctgaattaaaattagactttgaaaataaagcttaaatagttgtttcctctaaa

SEQ ID NO: 57 - protein, 114 aa, Homo sapiens
MAAIPSSGSLVATHDYYRRRLGSTSSNSSCSSTECPGEAIPHPPGLPKADPGHWWASFFFGKSTLPFMATVLESAEHSEPPQASSSMTACGLARDAPRKQPGGQSSTASAGPPS

SEQ ID NO: 58 - protein, 386 aa, Homo sapiens
MCSSVAAKLWFLTDRRIREDYPQKEILRALKAKCCEEELDFRAVVMDEVVLTIEQGNLGLRINGELITAYPQVVVVRVPTPWVQSDSDITVLRHLEKMGCRLMNRPQAILNCVNKFWTFQELAGHGVPLPDTFSYGGHENFAKMIDEAEVLEFPMVVKNTRGHRGKAVFLARDKHHLADLSHLIRHEAPYLFQKYVKESHGRDVRVIVVGGRVVGTMLRCSTDGRMQSNCSLGGVGMMCSLSEQGKQLAIQVSNILGMDVCGIDLLMKDDGSFCVCEANANVGFIAFDKACNLDVAGIIADYAASLLPSGRLTRRMSLLSVVSTASETSEPELGPPASTAVDNMSASSSSVDSDPESTERELLTKLPGGLFNMNQLLANEIKLLVD

SEQ ID NO: 59 - DNA, 5761 bp, Homo sapiens
  source 1..5761: mol_type = other DNA
gcggcaggcgagtgagggaacgaggagcggccgggtgtgagtgtgtgggagtgagagtgtgtgggagtggggtgagggagaagctgacgggacgcgaggctgtgagaaactgggcgagtgtgcgaggacgcccggccagcctgcgggagccgcagtcggcggaggagaaaggaggcggctcccggtatcccgaccccctccccctcctctccttcccccacttccagccgcccggcggcccgcgcttcctcgaaggccccagcccggctcagtcggccgagagcgagggaggagccccccgacccaggtgctggagtctgcttgtcagggaggggggcagtctgtgattctgagaacagagccaagaaggggaacagcaaattcagtcacagacaatcctccactcggtcaagagccacttttctcttcctgccttgcccccccgcagggggtaaggaactgagcgtttaatctttagccggttggctaccagctaaaattctacttatcttagtttctagtggatagctttcttattttgcccatgttttcttagaatccctgtttaatatacttttgtcagtagtagtatctaggagtagcagggagagtgacaataaattagccccttcttttttcccttgtcattcaggccccttttcctctccagagggaaattaccagtaaactcttctaaatcttccaccccttctcagtcatactgtgaagaaacacactaaagtggacattatttgaccagtgaacacgaacccagcttcaggcattggtttgttgtggcacatggagaaacatctcttttaaaatatctcccaattacccttttcacaatttgtatccacctaggatttgctgctggggtaagtcactagatttatttctcaaagctcccctctctatgagctgaaagactgaccaaccatgaacactagtaggggatggggaaaggggacagagcagagccagttgttccacactttgggaagcaggagtagcttttatcatcttcctctggggagcaggcatagagacataaactgagtgaaaatgggtggaggaagaacttctatacccacgaacaacatgtgaagagagagaaccaaacataaagtaaggagggtagacgttacatccaagaggaaataatccaggcaaggaagcacaagctgatcaagatgtgtagttctgtggctgccaagttgtggtttttgacagatcgtcgcatcagggaagactatcctcaaaaagagattttacgagcattgaaggccaaatgttgtgaggaggaactggactttagggctgtggtgatggatgaggtggtgctgacaatcgagcaaggaaacctgggtctgcggatcaatggagagctaatcactgcctacccacaagtggtggtagtcagagtaccaaccccttgggtgcaaagtgatagtgacatcactgttttgcgccatctagagaagatgggatgtcggttaatgaaccgacctcaagccatcctgaactgcgttaataagttctggacatttcaagagttggctggccatggtgttcctctgccggatactttctcttatggtggccacgaaaattttgctaaaatgattgatgaggctgaagttctggagttcccaatggtagtaaagaatacgcggggtcacagaggtaaagctgttttcttggctcgagataagcaccatttggctgatctaagccatcttattcgccatgaagcgccatacctgttccagaagtatgttaaagagtctcatggacgggatgtacgtgtcattgtcgtgggaggccgtgtggttggcaccatgttacgttgttcaacagatgggagaatgcaaagcaactgctcattaggtggtgtggggatgatgtgctcattgagtgaacaagggaagcagctagctatccaggtgtctaatatcctggggatggatgtgtgtggcattgatctgctgatgaaagatgacggctccttctgcgtctgtgaggccaatgcaaatgtaggtttcatcgcctttgataaggcttgtaatctagatgtagctggtatcatagcagactatgccgcctcccttctaccctctggccggctcacccggcgtatgtccctgctctccgtggtgtccactgccagtgagactagtgagccggagctgggtcccccagccagcactgctgttgacaacatgagtgcaagttccagctctgttgacagcgaccctgaaagcacggagcgagagctgctcaccaagctcccagggggcctgttcaacatgaaccagctgctagccaatgaaatcaaactactggtggactgactccactggtaattaaccaacaaaacccttgtaaaactttctttcttcttttctatttttaaaaccaacttgcaatgctgttcatggaggatgctcaggaagatgagagaaaattagtaggattagttggagagagtgggagatagatgagacctctgctagtaagatgttactttcatttacaaatcctacaaatagagaggcagaataggtggggtatagaaaaatgtcaggctctcatagttacccttttaaattgctaaaaaatgtgtatgctcataggccatgaggaacaaatactttttttttttcatggtcccttgcttttgtttttgtacaaaaaaaaatggttttgctacaaatatccaagtagcataacttcacattgtgttggaagatttgtcatcagtgaggaaaacatctgcataaattacaggaatttttgtattatacagctctgaaaattctgccatttccttattaactagcagctttagtttgtagtttatgaaatcttgaggggctcttttactgggatttcttatttttttgttttttcccgcttaatttggtgggaggtcaaattgaatataacccaataaaggcttcttaatgacaaaattggcatgtttgcatgatgaaatggaaatgaacagtattgcaatgtccggtatacaaaataacattaattcaatgtagataaaattacactagtttaaaatatgtgcattcacttgtatttgttagtgttttagtcttttttgaaagatgtgctctgttaatgttgcttttttttttttttttaatacatgctagtctaacatttcctgctctatgcctgcatctttaacaatggccaaagtgaagaaaatgctaccttttttgttaacaagacactgacttgaaacatgtacatttaaagccttttattttttccctttttgttttggtagttgggcatttaaataaggacaaggaaaaatatttttgggggcaaatcaagagcctatgagttctaagtataaagctgaagtgatttcgaatgccagcgttatatatttgcatttttcacattttacgagggagtatatgtgtatgtgtgtgcacgcatgcatgtgtatgtgttttgctttttgtttccatcaactaatcaaaaaggataatttagaaaatggagcatgatgggaaacagagtttttgactttaaaaaacagatgagttgttttcataagtagactccactggggtagaggtattcaccttaaaacatagggtgagtagatgcttttttaggcctttttgtgtatatgtacgttgtttgtttttttccttttgtttctagcctgttcagtgtacagtttattcaaggctacatgcttttctttaatgcttctggctatgcattttctctttttacatataggatttgggattgggggtgggttggatgtttttgtttggggacttatttagtagtattgagtctcttatagccctactcttaagccttcaatactgtccactctttatattcctttacttgcagaatttataaaagcccccaaactgcatataatatgagcctttaaaacatgggtaaaactaatcccattgatgggtttggatggtatgttaagaaatggagatgctgcagagcccaacgtaattttttaaacagcaagttttccatctccctacgaatcctctgaagcttttacccaagccctttcttgcctctccagtgctattttccttcagatggaccttaaacataatttcttggacactactagagagacttcgaggcaataataaaagatcagtattaaccagctataacagaggtttgatcatgcttacttgtacagtttttcccccgttttaaaaaggaatgtaataaaatttgttttttccatagaattaaataatattaaaattgagtgaaaggttgattgttgatgaatagaatagtacctctcatctgtgcagtgtctcatttcacctcagagaaaaggatacataagaggagtttgtaatttatcttaggatattctaattgcatttaaaagaacttatcttgcgcagggtaaatgggggactcacatacatatattaatacctctgactcattaacagaaagaaatacttggtacttctttcgctgaatgaccatactgtggaggatgcatactatttggtatagagaaataaatgaggaagaaagaactgcttaattaaattatcattcatatgttcatatagagaccatctggttgccatgtgtattatgacacatacactttgaatagttacatatcacaagtatgtagttcatgtttgtgttggtggggtaaggcatcaggaaaaatgtagttagtcttttcttaacttataccaaattaaccaactatattataggaaatatgtgaaattagttcattagctttattcactattatgcattcacatgatattaaaacgtacactcacatgttagaatgaaaagagcagtagttatcttagattttaaaaacatggatatcttcttgaattccttcaagattgaggtagagaataagagcaaatcattctggaagtaccttaaggaaacaaacagcagcagatatttaggttaaacttattttcataattgtttaataacttttgtataatcttcattgctattatgagagagaatgtatatatcaaatatgtgtaatgataaaatctgaattgtaaaatttttgtatattgttaaaattgtaattctaaattgtatttcaaaaatgattatttctgatattgtttttatgtcacccatgatgaaaactggactttatatatctaaacatacaagtatgaactattctatttaaaatttttaatagtttttttcttttttggtgcctataattgattggtcatttctgctggcttttctccaatgaacattgaaatcttcctgtatatgttaccaataagaaaactaccctggaacagtagaaaaacccaacaagagacttggcattcatcaagcacattatcagactttgagaacatattgaaggcattgactttgaaaatcatctctttttctcaagaagaaagcaatggagaagcaaatttgtttcattcagtgaatccccagtttggggcttgtggggcttagagacattgtgaaatcaaatcttgtgttatacttttctcctggctcactttttttgagaaggtttatgggctatttggctggtgagacacgatcccctcctaagaaaatgtaggtgctcagacaggtaaccactgctgctactgtttttatttgtttgtttgttcaattttatttaagatttgtttttgttgtactaggattttaaaaaatgtaatatattgcaggatttataaccagg

SEQ ID NO: 60 - protein, 226 aa, Homo sapiens
MAAAAVQGGRSGGSGGCSGAGGASNCGTGSGRSGLLDKWKIDDKPVKIDKWDGSAVKNSLDDSAKKVLLEKYKYVENFGLIDGRLTICTISCFFAIVALIWDYMHPFPESKPVLALCVISYFVMMGILTIYTSYKEKSIFLVAHRKDPTGMDPDDIWQLSSSLKRFDDKYTLKLTFISGRTKQQREAEFTKSIAKFFDHSGTLVMDAYEPEISRLHDSLAIERKIK

SEQ ID NO: 61 - protein, 322 aa, Homo sapiens
MLSPLPTGPLEACFSVQSRTSSPMEPPIPQSAPLTPNSVMVQPLLDSRMSHSRLQHPLTILPIDQVKTSHVENDYIDNPSLALTTGPKRTRGGAPELAPTPARCDQDVTHHWISFSGRPSSVSSSSSTSSDQRLLDHMAPPPVADQASPRAVRIQPKVVHCQPLDLKGPAVPPELDKHFLLCEACGKCKCKECASPRTLPSCWVCNQECLCSAQTLVNYGTCMCLVQGIFYHCTNEDDEGSCADHPCSCSRSNCCARWSFMGALSVVLPCLLCYLPATGCVKLAQRGYDRLRRPGCRCKHTNSVICKAASGDAKTSRPDKPF

SEQ ID NO: 62 - DNA, 4955 bp, Homo sapiens
  source 1..4955: mol_type = other DNA
gcaacatcgccgcggaggtagcgagctgagctgacagcgcggagctggcgctgtggagcgcagggagccttgccggttcctccgaccggcgtctgcgagtacagcggcggctaacctgccccggcttcaggatttacacagacgtggggcgatgcttgtgaccctgcagctcctcaaaccagcctgtattgagcggtttgcagcctgatgctcagccccctccccacagggcccctagaagcctgtttctccgtacagtccaggacctccagccccatggagcccccgatcccacagagcgcccccttgactcccaactcagtcatggtccagccccttcttgacagccggatgtcccacagccggctccagcacccactcaccatcctacccattgaccaggtgaagaccagccatgtggagaatgactacatagacaaccctagcctggccctgaccaccggcccaaagcggacccggggcggggccccagagctggccccgacgcccgcccgctgtgaccaggatgtcacccaccattggatctccttcagcgggcgccccagctctgtgagcagcagcagcagcacatcctctgaccaacggctcttagaccacatggcaccaccacccgtggctgaccaggcctcaccaagggctgtgcgcatccagcccaaggtggtccactgccagccgctggacctcaagggcccggcggtcccacccgagctggacaagcacttcttgctgtgcgaggcctgtgggaagtgtaaatgcaaggagtgtgcatccccccggacgttgccttcctgctgggtctgcaaccaggagtgcctgtgctcagcccagactctggtcaactatggcacgtgcatgtgtttggtgcagggcatcttctaccactgcacgaatgaggacgatgagggctcctgcgctgaccacccctgctcctgctcccgctccaactgctgcgcccgctggtccttcatgggtgctctctccgtggtgctgccctgcctgctctgctacctgcctgccaccggctgcgtgaagctggcccagcgtggctacgaccgtctgcgccgccctggttgccgctgcaagcacacgaacagcgtcatctgcaaagcagccagcggggatgccaagaccagcaggcccgacaagcctttctgacagtttgtgtcgaagccccagtgctctgcctggaaacctggttctcttctgacatctaagaagactgcagcaaggtcagaggttttagcctcctgaggctgaccttgctagtctgcccactccctacccccagcttcggaaaatacagagaccaccaccacgtaccctgtattccccaaggtgatgaagaagcactttggggctttttttcagggtcctgaaactttgtgtcaaacagacaatgcaggggcagggtgtggtttggggggaaatttttctttttcagaagacagaacacagatgtggacacatatccggaaactgcagctgcttgaatgccttcccagcccctccttctccctccctccctccgcccccccccccttcctcttttccattgtctttggctctcacaggagctagctgcctgggaggaattgttaactgagtaccagggtacctttaaagaagacccttggagtcttctataccttcttctccttccccatctcactccaccccactttgtccctgatgtcttggggaaggtgtagaacaccctagcagttcctattgtatatacttgggagccactgagaacagaggacggccagtgagtccaagcctcgttcctccttctgcctccccggagccacaggatggatttaggagccactgctcagtgcacttctcccttccaactgcatcaactaactctcgggggtgttctgctcaccacaccgtccttcggttcttactgagtcacagactcgcctgcccactacgtgtcctgggttctctctactcagatcccttccagaaactttatatgggtagaggaagccagggcggcaaatgcgagaccaaatatcattttgccaatgagtctgaggctgtggtctctggatccagtcattatgtttttatagaataattaaaccggatgctaacggtgttttaaaaaataataataaaacaacttgtttccttttggccacccccaggaagggctgatttcaaaatctgggggcgagcaacctcaaggaacacaatttccctccctatcaacaagaggattttaacagcaaagaagagaggcagcacctcccattggcagaatgaccgctgagccaggctgggtttgggtttcttctcttctgattctgctgctcactgtcatagccttttgtgtatagtgatgtgtctgtatctttaatgtaaatagagagatgatgaaaaaagagtctattttagtgttaggaagccccagcaggggagtcggaagagcttggaagagctggggagagggtaggggaaaggtttttccaggggccactgggtttgagccctgcttctgtgcacagccacaccaccctctcccgacagccctcaaagacgtagcaactctttctctcaaggtgctaaaggactcagaaggtgcagcacgtccagtgggtaggtacttgttgcatgcaaaagctgtagtgtatctggtccttcctccccagcttttgtgtggggttcttgctttgtgtggtattttgttttcccctctaatgagagggcatggcctgagtcagaagagctaccccaggtgaaactggaagtgcatgaggcagagcgtccgtagcatttccagtttgttctgtataggaacagaggtgcctccgggaaggaggcagcgaggtaggtagctatgataggcacctaatgcttctcaaggacttattttttccttcttgaagactagtagtaacatcttatgatttagagtaagttgattgtaaccataggtatttattgattggaggaagggagggtcatattattttcggctttatttatgtaacatttgctagcttgtaaaaggcgaatgtgaaatattgcatctgcattttccaaggctgattcgtgtagctacccttgccacagttgtgacggatgtatggatgttcttgaacatttcagaaggagtggtagaaaaaaacacacattcagccaaccacttatatgaattgaatgtatcagaagtgtactgaagggactggagatggttttcctcagatgagggggccccaaaattgatagtgcacatctgcacgctttctgcgaggcctcagaacttcccagggcccctccctcaaattgtctccatgggaaacttgacccagtggcaagttgcactttggtgatcttggtggtctacacacccgttctgtggagagtcgatttacataagctgtgtatacacacacacacacacacacacacacccctaccccacactgactgtctaccgacaaagaccctatttcctggcaaacggcctcctgaaccctgactttttgtgtacatacttgtaaacacggatttttctgggttttggtttgctttttccttttttccccctgcccctgttctagcttgttcttcttggtttgctttcaacctgcttgatggatgtctgcagagtgctctctaagagtccacctcagtgcctcgtgtgctcagtggtcatgggaaaggagcgaaggaaccatccttggttctcccagcttggttgtgtagcaatccctcagcattgtttttctcagcttcttggcaaaaattaaaacaacaacaacaacaacaacaacaacaacaaacagaaggataaactggcttgcctgtggaccctccccggctctggggccagtcgagagccactgagggacccagcactcagagacacaacacacatgtgtagctgcttctggctgagtgtgtttcctgtcaccaatggcctgtttggctggacgatgcctcggcttgaccttttttgaaaagtgctggttagttcccgcccctggtaaacctggggtaggtgggggttctgtcttaactcgaggggcacctgggatccaggacgcttctagggggctctggctgcccgtgttaatgaaggacagcgcttccgcgagcaccctgggaactgggtcttgggtagcaaagccctcccagagaaaagattgggcacaactaaggctttcctgagcaggaagggggtgaagaccaatcccttcctttggtcctttggtacgcaccccctcagagctgagatggaagacatggctagttcttttcagccttgtggagcctgtcagtcgccatcatacctcgagtgaggcccagctagataatgacttgtccaagatggcacacgtggaaagttgatctgcaccagaacccggatgactgtcaccttgaagcatcctgttctccttctgtgctgtcccaggaagtgtctggcgggcgtgggcagcacagctctacactgtacgattcactagggcatcctgcgagcctcactagccttctggttcatgcctttgacaagcatttttgtgccccctctgcttactgtgacagtcgatgatgaatcttgcgttgccattttctgctgtgggtaactgcgtgcagtgtcttgccttgctttctcttcttactgtcccacagcttggtttcatgttacaaacagaaaagctcgaggctcccaccccgccacatcccaacttcatttccccctcactgtagcccatttccaccccaccacaaagttgccacaggttttctttgtatagaatatttattttgaagctctattttaatagtatttattttagaaagtctactattgtaagagttcttctgtttgtgaagaaaaaaacaagttaaaaactgaatgtactgatttagaaaatatatataaatatatattgttaaatatacacgggactgcc